(19) United States
(12) Patent Application Publication
(10) Pub. No.: US 2023/0030539 A1
(43) Pub. Date: Feb. 2, 2023

(54) METHOD FOR ANALYZING THE METABOLIC CONTENT OF A BIOLOGICAL SAMPLE

(71) Applicant: BASF PLANT SCIENCE COMPANY GMBH, Ludwigshafen (DE)

(72) Inventors: Elie Fux, Gauting (DE); Sandra Gonzalez Maldonado, Ludwigshafen (DE); Michael Herold, Berlin (DE)

(21) Appl. No.: 17/782,290

(22) PCT Filed: Dec. 4, 2020
(86) PCT No.: PCT/EP2020/084634

§ 371 Date: Jun. 3, 2022

(30) Foreign Application Priority Data
Dec. 5, 2019 (EP) 19213900.4

(51) Int. Cl. G16B 40/10; G01N 33/68; G16H 50/20; G16H 10/40

(52) U.S. Cl. CPC G16B 40/10; G01N 33/6848; G16H 50/20; G16H 10/40

(57) ABSTRACT

The invention relates to a method of analyzing the metabolic content of a biological sample comprising: i) providing one or more samples of extracted metabolites from the biological sample; ii) performing a chromatography coupled mass spectrometry analysis of the extracted metabolites to generate a full raw data set for full scan ions; iii) generating a full data cluster set from the full raw data set obtained in step ii) by grouping full scan ions according to isotope and adduct values; iv) performing a tandem mass spectrometry analysis of the extracted metabolites with a plurality of mass selection windows to generate a raw SWATH® data set for fragment ions; v) generating a SWATH® data cluster set from the raw SWATH® data set obtained in step iv) by grouping fragment ions according to retention time and mass values; vi) aligning the SWATH® data cluster set with the full data cluster set to generate characteristic profile for each extracted metabolite; vii) comparing the data using R characteristic profile of each extracted metabolite obtained in step vi) with a reference library of characteristic profiles of metabolites to provide the metabolic content of the biological sample.

### BACKGROUND

The present invention relates to a method of analyzing the metabolic content of a biological sample comprising: (i) providing one or more samples of extracted metabolites from the biological sample; (ii) performing a chromatography coupled mass spectrometry analysis of the extracted metabolites to generate a full raw data set for full scan ions; (iii) generating a full data cluster set from the full raw data set obtained in step ii) by grouping full scan ions according to isotope and adduct values; (iv) performing a tandem mass spectrometry analysis of the extracted metabolites with a plurality of mass selection windows to generate a raw SWATH® (registered trademark of AB SCIEX, LLC) data set for fragment ions; (v) generating a SWATH® data cluster set from the raw SWATH® data set obtained in step iv) by grouping fragment ions according to retention time and mass values; (vi) aligning the SWATH® data cluster set with the full data cluster set to generate characteristic profile for each extracted metabolite; (vii) comparing the characteristic profile of each extracted metabolite obtained in step vi) with a reference library of characteristic profiles of metabolites to provide the metabolic content of the biological sample. Further the present invention relates to methods related to said method.

Metabolite profiling, often called “metabolomics”, is a powerful tool of choice for a wide range of high value, precise and fast diagnostic applications as well as for discovery in the pharmaceutical and nutritional fields. Metabolomics is the study of metabolites in a biological sample. Within the context of metabolomics, generally a metabolite is usually defined as any molecule less than 1 kDa in size. Collectively, these small molecules and their interactions within a biological system are known as the metabolome.

Metabolites are the products or intermediates of biochemical pathways and cellular mechanisms. The precise number of metabolites in many organisms is unknown. Estimates in, for example, humans range from about 2,000 to as many as 20,000 different metabolites. Of particular interest are the so-called small molecules, i.e. low-molecular weight compounds that serve as substrates, intermediates or products of the various metabolic biochemical pathways. Whereas genes and proteins mostly predetermine what happens in the cell, much of the actual biological activity happens at the metabolite level, including cell signaling, energy transfer, and cell to cell communication, all of which are also regulated by metabolites. Accordingly, although genes and proteins are closely linked to cellular mechanisms, metabolites even more closely reflect the actual cellular activities in response to endogenous factors, e.g., signaling between different cells, or exogenous factors, e.g., changes in environmental conditions. Thus, changes in the metabolome are the ultimate answer of an organism to genetic alterations, disease, or environmental influences. The metabolome is, therefore, most predictive for a phenotype. Consequently, the comprehensive and quantitative study of metabolites (i.e. metabolomics) is a desirable tool for studying various endogenous and exogenous effects on an organism's phenotype and, thus, complex biological issues relating to, e.g., disease development and progression or toxicity can be efficiently addressed. As mentioned before, an advantage of metabolomics is that the effects caused by exogenous factors can be immediately monitored by metabolic changes which usually appear much earlier than changes in the transcriptome, proteome or even the genome or epigenome of an organism, if any. Metabolomics allows the determination of effects of exogenous factors which do not influence the genome, transcriptome or proteome of an organism immediately. For instance, a toxic compound may be harmful for an organism but may not necessarily cause changes in the genome of said organism.

Metabolite profiling can be used for a wide variety of purposes. For example, from product and stress testing in food industries, e.g. control of pesticides and identification of potentially harmful bacterial strains, to research in agriculture (crop protection and engineering), medical diagnostics in healthcare, and future applications in personalized medicine resulting in personalized treatment strategies.

The possibility to discover novel metabolic markers as well as the potential to monitor highly complex metabolic networks has been made possible by breakthroughs in modern analytic technologies. It has been driven by quantum leaps in computing and bioinformatics, embracing data validation, data processing, data clustering and data integration. Bioinformatics allows the reliable interpretation of complex metabolic patterns and novel markers can be identified fast and with high precision.

Various techniques are known for the analysis of complex mixtures of compounds such as the metabolome of an organism. These techniques include, for instance, mass spectrometry, tandem mass spectrometry, nuclear magnetic resonance (NMR), Fourier transform infrared (FT-IR) spectrometry, and flame ionisation detection (FID), optionally coupled to chromatographic separation techniques such as liquid chromatography, gas chromatography or high performance liquid chromatography (HPLC).

However, there remains significant problems for the identification of the metabolome of a biological sample. One such issue is that metabolite identification is a major bottleneck for metabolomics analysis.

Despite the use of modern analytical tools, such as chromatography coupled with high-resolution mass spectrometry, the identification of the vast majority of the observed peaks in any one sample remains unknown. For example, for the same retention time, exact mass and molecular formula there can be multiple, sometimes hundreds, of potential chemical structures. These potential structures can be provided as only a tentative list(s) of metabolite identifications.

### SUMMARY OF THE INVENTION

Against this background the present invention provides a method which allows for the quantitative analysis of all detectable metabolites present in a biological sample which clearly provides an important resource for determining the metabolome of that sample.

A first aspect of the invention provides a method of analyzing the metabolic content of a biological sample comprising:
i) providing one or more samples of extracted metabolites from the biological sample;
ii) performing a chromatography coupled mass spectrometry analysis of the extracted metabolites to generate a full raw data set for full scan ions;
iii) generating a full data cluster set from the full raw data set obtained in step ii) by grouping full scan ions according to isotope and adduct values;
iv) performing a tandem mass spectrometry analysis of the extracted metabolites with a plurality of mass selection windows to generate a raw SWATH® data set for fragment ions;
v) generating a SWATH® data cluster set from the raw SWATH® data set obtained in step
iv) by grouping fragment ions according to retention time and mass values
vi) aligning the SWATH® data cluster set with the full data cluster set to generate characteristic profile for each extracted metabolite;
vii) comparing the characteristic profile of each extracted metabolite obtained in step vi) with a reference library of characteristic profiles of metabolites to provide the metabolic content of the biological sample.

The method of the invention provides a method of analyzing the metabolic content of a biological sample.

The expression “method for analyzing” means that the method of the present invention may be used for all analytical purposes. The method of the invention may essentially consist of the aforementioned steps or may include further steps. Moreover, it is further envisaged that the method of the present invention may be itself included into methods for different purposes such as screening methods, diagnostic methods or quality control methods. Preferred technical fields in which the method of the present invention can be applied are described in detail below.

The method generates two data sets from a biological sample. One data set is generated using chromatography coupled mass spectrometry analysis and provides an inventory of the detectable metabolites in a sample, termed the “full raw data set for full scan ions”. A further dataset is generated from the same sample using tandem mass spectrometry with DIA (data-independent acquisition) with SWATH® MS, termed the “raw SWATH® data set for fragment ions”. The two datasets are then individually grouped and subsequently aligned together. The aligned result provides characteristic profile for each extracted metabolite and specific metabolites identified by data mining from reference libraries of characteristic profiles of metabolites.

This method of the invention allows for the quantitative analysis of all detectable metabolites present in a biological sample which clearly provides an important resource for determining the metabolome of that sample. The method of the invention also allows a subset of metabolites to be identified which can be robustly applied to multiple differing biological samples in a HTP workflow.

A key development of the method of the invention is the integration of the two data sets, i.e. the “full raw data set for full scan ions” with the “raw SWATH® data set for fragment ions”. This is achieved by (a) generating a full data cluster set from the full raw data set by grouping full scan ions according to isotope and adduct values, and (b) generating a SWATH® data cluster set from the raw SWATH® data set by grouping fragment ions according to retention time and mass values. The full data cluster set is then aligned and integrated with the SWATH® data cluster set to generate a characteristic profile for each extracted metabolite.

By assigning one ion or a group of full scan ions of the full raw data set to a set of fragment ions of the raw SWATH® data set without prior knowledge of the chemical structure of the metabolite of origin, the method of the invention allows metabolites to be unambiguously defined by their chromatographic and mass spectrometric parameters: exact mass to charge ratios of related full scan ions, retention time, the exact mass to charge ratios of fragment ions and the intensities of these fragment ions. The characteristic profile for each of the extracted metabolites, consisting of those parameters may be used to compare with characteristic profiles of metabolites from other samples, for comparison to those values for metabolites of known identity in one or more reference libraries and/or to set up a high throughput method for quantification.

A further development of the invention is the use of SWATH® with narrow selection window of precursor ion masses, preferably around 1 Da.

Many metabolites have only a small difference in their molecular masses and hence cannot be distinguished from each other with a broad SWATH® selection window of precursor ions. Therefore, they cannot be individually assigned to their related fragment ions and comparison and identification from their characteristic profiles will fail, if the metabolites are not well separated by chromatography, which is a challenge in a complex metabolomics sample. From this perspective, a narrow SWATH® resolution window aids the assignment of individual fragment ions to full scan ions and hence metabolite identities. It allows the rapid identification of chromatographically unseparated metabolites with similar but not identical masses on the scale of 1 Da precurser ion selection window, which is necessary for achieving identification of metabolites with similar masses in complex samples.

Existing methods of metabolite identification in metabolome analysis usually utilize mass spectrometry to deal with small sample amounts coupled to a chromatographic method to separate complex samples. Usually high resolution tandem mass analyzers such as Time-of-Flight or FT-MS technology are used to get information on metabolites as precise as possible from small sample amounts. Only DIA MS/MS experiments such as SWATH® MS (which is a method well known in art; for example, see: https://sciex.com/technology/swath-acquisition) can generate qualitative results for identification as well as quantitative results used in the method of the invention to calculate a simple linear regression model to evaluate correlation of amount of the metabolite with sample amount. DIA MS/MS experiments like SWATH® MS either with fixed or variable precursor ion selection windows for untargeted metabolomics analyses in order to find and identify metabolites are used usually with precursor ion mass selection windows of 20 Da and more and they therefore have the disadvantage of not fully resolved signals from different metabolites in complex samples, as explained previously herein.

A further state of the art method, using SWATH® MS in combination with a deconvolution algorithm (Tsugawa H, Cajka T, Kind T, Ma Y2 Higgins B, Ikeda K, Kanazawa M, VanderGheynst J, Fiehn O, Arita M; MS-DIAL: data-independent MS/MS deconvolution for comprehensive metabolome analysis. Nat Methods. 2015 June; 12(6):523-6. doi: 10.1038/nmeth.3393. Epub 2015 May 4.) in order to resolve and identify chromatographically good as well as partially separated metabolites, does not provide full deconvolution of metabolite ions from metabolites with very similar retention times and similar masses and allows not for their identification.

In contrast to the methods in the art, the method of the present invention allows for a timely and accurate identification of the metabolic profile of a biological sample.

The invention will now be described according to the features and the methodology used.

The following figures illustrate the present invention.

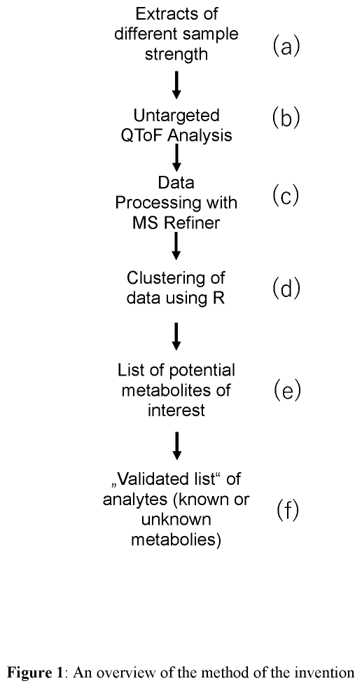

FIG. 1: An overview of the method of the invention.

In FIG. 1 (a) to (f) have the following meaning:

(a) Extraction of matrix of interest e.g. leaf, plasma, cells, urine. Different volumes or weights of matrix extracted in order to create a “calibration” curve. The target weight or volume for high-throughput method is performed in triplicate others with one replicate.

(b) Produce high resolution full scan and MS/MS data from 100 to 1000 Da with 1 Da precursor ion intervals. QToF acquisition on-line with chromatographic separation (HPLC, different types, e.g. reversed phase and HILIC in subsequent experiments or multiplexed in one experiment using column switching). QToF acquisition performed with ESI+ and ESI− ionisation.

(c) Retention Time correction. Mass correction. Group full scan features into isotope clusters and adduct groups. Library annotations.

(d) Cluster MS/MS features from each precursor selection window together according to retention time (hierarchical clustering). Assign MS/MS features to full scan precursor features based on mass and retention time. Produce aggregated list of all features with group Ids for all MS/MS features assigned to full scan features.

(e) Assess quality of groups using results of correlation of signals to weights or volumes of matrix as well as variability from the replicate at the target concentration. Confirm the grouping of known metabolites present in library by number of annotated peaks (full scan and MS/MS). Explore non annotated features groups and include in library if confirmed as new unknown compound.

(f) Use the validated list of analytes to create high-throughput method on QqQ MRM parameters can be optimized when known substances are available as standard. When the analyte is derived from unknown metabolite or a metabolite that is not available as standard, the result of the clustering can be used to deduct the MRM (precursor and fragment mass). Retention times on QqQ are obtained from the validated list as identical chromatography are used.

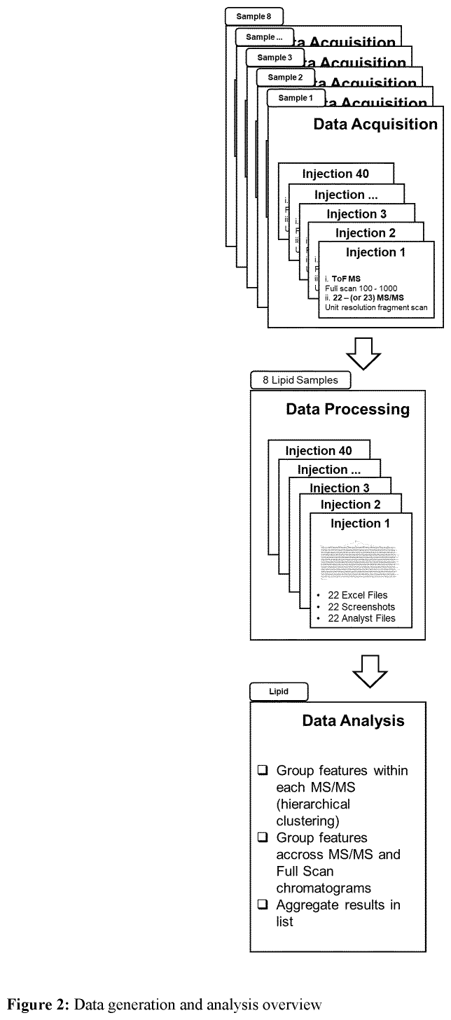

FIG. 2: Data generation and analysis overview.

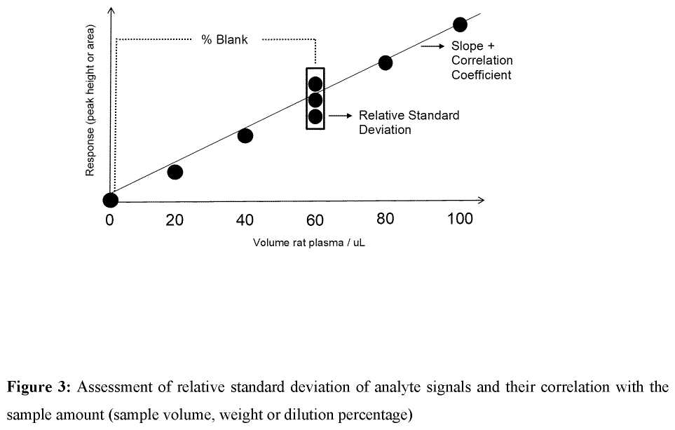

FIG. 3: Assessment of relative standard deviation of analyte signals and their correlation with the sample amount (sample volume, weight or dilution percentage).

### DETAILED DESCRIPTION AND DEFINITIONS

The terms of “grouping” and “clustering” as well as “to group” or “to cluster” are used in a synonymous manner for steps where features or related detected ions are combined to a bigger entity by a specific relationship. This relationship may be based on similar or common properties or based on the same origin of those features or related ions. The result may synonymously be called a “group” or a “cluster”, independently of the type of the relationship, which the combination step is based on. The particular type of that relationship can be understood from the context of the usage of those terms. For example, the underlying relationship may be based on the consideration, features or ions are related to different isotopologues of the same compound. The relationship may be that ion species are considered to be different adducts formed during the ionization process from the same compound. The relationship may also be that fragment ions from MS/MS experiments are generated from the same precursor mass selection window and elute with a similar retention time from the chromatographic column, indication that those MS/MS-ions are related to the same compound. Furthermore, the relationship may be that those MS/MS-ions from the same precursor mass selection window and with similar retention time have similar retention time also to a MS full scan ion which fits into the same precursor mass selection window, indicating, that the MS full scan ion is the precursor ion of those fragments and all are related to the same compound.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way.

The invention may, as the skilled person will recognize, be performed by using alternative features. Similarly, features introduced by “in an embodiment of the invention” or similar expressions are intended to be optional features, without any restriction regarding further embodiments of the invention, without any restrictions regarding the scope of the invention and without any restriction regarding the possibility of combining the features introduced in such way with other optional or non-optional features of the invention. Moreover, if not otherwise indicated, the term “about” relates to the indicated value with the commonly accepted technical precision in the relevant field, preferably relates to the indicated value ±20%, more preferably ±10%, most preferably ±5%.

Metabolite Separation and Data Generation

The method of the invention comprises two steps in which data acquisition relating to the extracted metabolites is performed. One step, listed in the method as step ii), generates a full raw data set for full scan ions for the extracted metabolites in the sample using chromatography coupled mass spectrometry analysis. Here the metabolites are extracted and temporal (retention time), mass to charge ratio (m/z) and intensity data are gathered. The data relates to “full scan ions”, and this stage generates a large complex data set containing noise as well metabolite specific ions, which is difficult to resolve.

A separate step, listed in the method as step iv), is a tandem mass spectrometry analysis of the extracted metabolites. Here metabolite-derived ions (called full scan ions and because used for fragmentation also called parent or precursor ions) are selected or filtered within mass selection windows, followed by fragmentation of the parent ions and retention time and mass to charge ratio (m/z) and intensity data is gathered for the fragments. This step generates multiple data parameters for each parent ion. By combination of the two data sets, it is possible to determine the composition of the extracted metabolites from the biological sample.

It is important to point out that step ii) and step iv) are performed independently of each other. Moreover, there is no specific order of which step is performed which is important to the invention. In other words, step ii) can be performed before step iv), or step iv) before step ii), or both simultaneously. However, for data analysis, alignment and integration of both data sets it is of big advantage to use the same chromatography and that extracted metabolites elute at same retention time when generating both datasets.

Nonetheless, where a single apparatus is used for steps ii) and iv), it is possible to perform both data acquisition stages from a same sample. Again, there is no specific order of which step is performed which is important to the invention. In both steps mass to charge ratio (abbreviated as m/z) is a parameter specific for an ion species, deriving from a metabolite (without or with fragmentation of a precursor ion). As ions deriving from small molecules by the ionization processes applied by the method of the invention are usually singly charged, the term “mass” is equivalently used for m/z, because a person skilled in the art knows, that for singly charged ions m/z relates to mass just by a constant factor of an elementary charge unit. The mass unit Da (Dalton) is therefore used for m/z as well. The term fragment data may also refer to a fragment ion mass, because the mass is the most important feature if an ion in mass spectrometry.

a) chromatography coupled mass spectrometry analysis

The method of the first aspect of the invention includes step ii) in which a chromatography coupled mass spectrometry analysis of the extracted metabolites is performed.

The term “chromatography coupled mass spectrometry” as used herein relates to mass spectrometry which is coupled to a prior chromatographic separation of the compound(s) comprised by the samples to be investigated.

Chromatography is a laboratory technique for the separation of a mixture. The mixture is dissolved in a fluid called the mobile phase, which carries it through a structure holding another material called the stationary phase. The various constituents of the mixture travel at different speeds, causing them to separate. The separation is based on differential partitioning between the mobile and stationary phases. Subtle differences in a compound's partition coefficient result in differential retention on the stationary phase and thus affect the separation.

The “retention time” is the characteristic time it takes for a particular analyte to pass through the system (from the injection unit through the column to the detector) under set conditions. Hence chromatography is used to assign a specific retention time to a specific metabolite in the analyzed sample.

Suitable techniques for separation to be used preferably in accordance with the present invention, therefore, include all chromatographic and/or electrophoretic separation techniques such as liquid chromatography (LC), high performance liquid chromatography (HPLC), ultra performance liquid chromatography (UPLC), gas chromatography (GC), thin layer chromatography, size exclusion, affinity chromatography and capillary electrophoresis (CE). Most preferably, GC, LC, UPLC and/or HPLC are chromatographic techniques to be envisaged by the method of the present invention. Suitable devices for such determination of analyte(s) are well known in the art.

Following the chromatography stage, the metabolites are then analyzed by mass spectrometry.

Mass spectrometry (MS) is an analytical technique that ionizes chemical species and sorts the ions based on their mass to charge ratio (m/z) and detects the ion current intensity or ion count related to this specific m/z. In one example, MS gathers ion counts or measures signals related to the amounts of different ions, where the difference of those ions is based on their different m/z. Sorting by m/z can, for example, be done by electrical and/or magnetic fields. This process of sorting may happen in time and/or space, where time or space or a combination thereof and knowledge of applied fields may be used for the determination of m/z of detected ions. In another example of MS function, this information can be gathered by measuring voltages or currents, induced by moving ions, where the movement is caused by electrical and/or magnetic fields. Mass spectrometry is used in many different fields and is applied to pure samples as well as complex mixtures.

A mass spectrum is a plot of the ion signal as a function of the m/z, where the ion signal is a numeric value, which refers to the amounts of detected ions related to the corresponding m/z. These spectra are used to determine the elemental and/or isotopic signature of a sample, the masses of particles and of molecules, and to elucidate the chemical structures of molecules, such as peptides and other chemical compounds, as well as the relative amount of different chemical compounds within a sample.

In a typical MS procedure, a sample, which may be solid, liquid, or gas, is ionized, for example by proton transfer or bombarding it with electrons. This may cause some of the sample's molecules to be converted into charged ions, termed “full scan ions”. These full scan ions are then separated according to their m/z, typically by accelerating them and subjecting them to an electric and/or magnetic field: full scan ions of the same m/z will undergo the same amount of deflection. The full scan ions are detected by a mechanism capable of detecting charged particles, for example an appliance including an electron multiplier. Results are displayed as spectra of the relative abundance of detected full scan ions as a function of the m/z. Hence mass spectrometry is used to assign one or a group of specific m/z of an ion or ions to a specific metabolite or a mixture of metabolites in the analyzed sample, due to the ionization process.

Mass spectrometry as used herein encompasses all techniques which allow for the determination of the molecular weight (i.e. the mass) or a mass variable corresponding to a compound to be determined in accordance with the present invention. Preferably, mass spectrometry as used herein relates to GCMS, LC-MS (where LC can be different types of liquid chromatography, such as HPLC or UPLC), direct infusion mass spectrometry, FT-ICR-MS, CE-MS, HPLC-MS. How to apply these techniques is well known to the person skilled in the art. Moreover, suitable devices are commercially available. More preferably, mass spectrometry as used herein relates to LC-MS and/or GC-MS.

Step ii) generates a “full raw data set for full scan ions”. The raw data set provides intensity, retention time data and mass to charge ratio (m/z) data for full scan ions, and hence extracted metabolites.

As can be appreciated by the skilled person, chromatography coupled mass spectrometry measures ion impacts. The subsequently generated electrical signal will then be transformed into a full raw data set based on the intensity value of said signal and a mass-related value, resulting from parameters such as time of impact of ions at the detector and/or position of impact (channel position) and/or knowledge of fields applied and/or fields measured.

Therefore, where mass spectrometry is used, the full raw data set is characterized by a m/z variable and an intensity variable. Moreover, since the method of the invention uses chromatography, each data point of the full raw data set also comprises a retention time, which hence is considered a third variable in this data set.

It is to be understood further that a metabolite may produce more than one data point in the full raw data set. Where mass spectrometry is used, data points may result in peaks by aggregation of data points of the typical distribution of the intensity over the m/z value of an ion species (depending on the resolution of the mass spectrometer).

Accordingly, if in a preferred embodiment of the present invention LC-MS and/or GC-MS is used for metabolite determination, the primary data points for a metabolite have also a typical intensity distribution over the chromatographic retention time. For the generation of peaks in the full raw data set, it is preferred to aggregate the data points over the retention time variable as well. Data points are processed in a three dimensional format within a sample. Said format has a retention time variable range, a m/z variable range and an intensity variable range. The format contains data points corresponding to the measured ion signals. The entirety of the data points will build up a three dimensional landscape comprising maxima (i.e. peaks) and minima (i.e. zero level data points for the intensity variable) of the intensity variable over retention time and m/z variables. After aggregating the primary data points to peaks, peaks are at least characterized by m/z and retention time of the peak maximum, a related intensity value and further information, e.g. extracted sample they are related to. In a preferred embodiment of the invention the data points intensities are aggregated over retention time and m/z for individual ion species of metabolites that are well separated or at least partly separated in retention time and m/z. It is to be understood that the full raw data set may be also presented by other suitable formats such as data sheets.

It is to be understood further that a metabolite may produce more than one peak in the full raw data set.

An “intensity variable” as used herein in relation to all embodiments of the invention, may be any variable which reflects a measured signal intensity. The signal intensity, preferably, directly or indirectly correlates with the abundance of a compound.

Preferably the full raw data set provides intensity data, retention time data and m/z data for measured full scan ions, and hence extracted metabolites.

b) tandem mass spectrometry analysis

The method of the first aspect of the invention includes step iv) in which a tandem mass spectrometry analysis of the extracted metabolites is performed.

Tandem mass spectrometry, also known as MS/MS or MS², involves multiple steps of mass spectrometry selection, with some form of ion fragmentation occurring in between the stages. In a tandem mass spectrometer, ions are formed in the ion source and separated by m/z in the first stage of mass spectrometry (MS1). Ions of a particular m/z range (known herein as “precursor ions”) are then selected (derived term: selection window) and “fragment ions” (also known herein as “product ions”) are created by collision-induced dissociation, ion-molecule reaction, photodissociation, or other process. The resulting ions are then separated and detected in a second stage of mass spectrometry (MS2).

Tandem mass spectrometer can include one or more physical mass analyzers that perform mass analyses. A mass analyzer of a tandem mass spectrometer can include, but is not limited to, a time-off-light (TOF), a triple quadrupole, an ion trap, a linear ion trap, an orbitrap, or an Ion Cyclotron Resonance mass analyzer. Tandem mass spectrometer can also include a separation device. The separation device can perform a separation technique that includes, but is not limited to, liquid chromatography, gas chromatography, capillary electrophoresis, or ion mobility. As an alternative, ion mobility can be used in combination with liquid chromatography separation techniques.

Tandem mass spectrometer performs a plurality of fragment ion scans one or more times across a mass range using a plurality of mass selection windows. The plurality of fragment ion scans are performed in a single sample analysis. A single sample analysis is, for example, a single sample injection. From the plurality of fragment ion scans (also known as product ion scan), tandem mass spectrometer produces all sample fragment ion spectra of all detectable compounds for each mass selection window.

Step iv) generates a raw SWATH® data set for fragment ions.

SWATH® is a data-independent acquisition (DIA) method which allows a complete and permanent recording of all fragment ions of the metabolite derived precursor ions present in a biological sample. SWATH® allows dynamic quantitative target transitions and modified forms of the target compounds (such as metabolites or post-translational modifications) to be determined without re-acquiring data on the sample. Since the LC-MS acquisition can cover the complete analyte content of a sample across the recorded mass and retention time ranges the data can be analyzed at any time to determine the metabolic composition of the sample.

The method is described as follows. In DIA (which is a method well known in art; for example, see: https://en.wikipedia.org/wiki/Data-independent_acquisition), the mass spectrometer settings will vary from experiment to experiment, depending on the specific apparatus used (e.g. speed of the chromatography apparatus) and objective sought (e.g. the mass range of interest). In general, the setting steps for example within 0.5-4 seconds cycle time through a set of precursor ion mass selection windows designed to cover 400-1200 m/z as a whole mass range readily covered by a quadrupole mass analyzer. During each cycle, the mass spectrometer thus fragments all precursor ions from the quadrupole mass selection window (which is the same as a precursor ion mass selection window) and records a complete, high accuracy fragment ion spectrum of all precursor ions selected in that mass selection window. The same precursor ion mass selection window is fragmented over and over at each cycle during the entire chromatographic separation, thus providing a time-resolved recording of the fragment ions of all the metabolite-derived precursor ions that elute on the chromatography. The SWATH® MS data consists therefore of highly multiplexed fragment ion maps that are deterministically recorded over the user defined precursor ion mass range and chromatographic separation.

The format contains data points corresponding to the measured fragment ion signals. The entirety of the data points will build up a three-dimensional landscape comprising maxima (i.e. peaks) and minima (i.e. zero level data points for the intensity variable) of the intensity variable over retention time and m/z variables within a precursor ion mass selection window and within a sample. After aggregating the primary data points to peaks, peaks are at least characterized by m/z and retention time of the peak maximum, a related intensity value and further information, e.g. precursor ion mass selection window and extracted sample they are related to.

Previous uses of SWATH® used precursor ion mass selection windows of around 25 Da wide mass ranges. This large window range was used since existing methods of SWATH® MS analysis are directed to proteomic analysis of biological samples. However, the present method of the invention is directed to metabolites and not proteins.

As discussed above, many metabolites have only a small difference in their relative masses and hence cannot be distinguished from each other with a broad SWATH® selection window of precursor ions. From this perspective, a narrow SWATH® resolution aids the assignment of individual fragment ions to precursor ions and hence specific metabolites. It allows the rapid identification of chromatographically unseparated metabolites with similar masses which is necessary for achieving good separation of all metabolites with similar masses.

Hence a preferred embodiment of the invention is the use of SWATH® with narrow selection window of precursor ion masses, preferably less than approximately 5 Dalton, preferably less than 4, 3, 2 Daltons, most preferably approximately 1 Dalton (Da). Such an embodiment is conducted as follows. An injection aliquot of extracted metabolites is separated by chromatography. The separated metabolites are then subjected to MS/MS analysis. Each injection aliquot of extracted metabolites can provide MS/MS data with 22 or 23 SWATH® windows (m/z range of precursor ion mass selection) of 1 Da. Hence, if the method is to provide a raw SWATH® data set for all metabolites having a size range of 100 Da to 1000 Da, around 40 separate tandem mass spectrometry analyses of the extracted metabolites should be conducted to provide the raw SWATH® data set for fragment ions in a 1 Da SWATH® window.

To perform a SWATH® analysis, providing a raw SWATH® data set using a window of approximately 1 Da, it may be necessary to make adjustments to the MS/MS apparatus used.

A further embodiment of the invention is the use of single and/or multiple (variable or discrete) collision energies for each mass selection window. The increasing or decreasing fragment ion intensities acquired during such multiple collision energy experiments can strengthen the identification of fragment ion peak groups that originate from the same precursor ion.

As can be appreciated by the skilled person and as discussed further above, tandem mass spectrometry analysis measures ion impacts. The subsequently generated electrical signal will then be transformed into raw data set based on the intensity value of said signal and a m/z value, such as position of impact (channel position), mass filter settings or time until impact, as well as a m/z of the precursor ion selection step.

Step iv) generates a raw SWATH® data set for fragment ions. In an embodiment of the method of the invention the raw SWATH® data set comprises m/z, retention time and intensity data.

Data Analysis

The method of the invention comprises two data generation steps: one step, listed in the method as step ii), generates a full raw data set for the extracted metabolites in the sample using chromatography coupled mass spectrometry analysis.

The other step listed in the method as step iv), is a tandem mass spectrometry analysis of the extracted metabolites and provides a raw SWATH® data set for fragment ions.

Following data acquisition, the method of the invention then performs a series of data analysis steps. These are described below.

The method of the invention uses data analysis techniques as described below that are implemented on a computer system, with elements including processor, data storage, and input/output devices and connections as known to a person of skill. While features of the data analysis techniques are implemented in software on a computer readable medium, a person of skill, with reference to this description, can prepare the appropriate computer-readable code for a computer system on which the embodiment is implemented, and as such software code and pseudo-code is not provided herein. It will be appreciated that various hardware and/or software combinations may be used to implement different embodiments.

a) Generating a Full Data Cluster Set from the Full Raw Data Set

The method of the invention includes in step iii) the generation of a full data cluster set from the full raw data set.

As outlined above, in step ii) a chromatography coupled mass spectrometry analysis of the extracted metabolites generates a full raw data set for full scan ions. That full raw data set comprises intensity data, retention time data and m/z data for measured full scan ions.

However, it is known and understood in the art that variations in mass may occur between full scan ions derived from the same metabolites. These mass variations are predominately caused by isotopic variations between full scan ions and adduct formation during the mass spectrometry analysis.

Isotopic variation between full scan ions occurs due to the presence of different isotopes for common elements in nature. For example, oxygen (three isotopes), sulphur (four isotopes), iron (four isotopes), calcium (six isotopes), carbon, nitrogen and chlorine. As can be appreciated, full scan ions derived from the same metabolite may differ in mass according to whether they incorporate different isotopes. Hence, to generate a full data cluster set from the full raw data set, full scan ions are grouped according to isotope values.

An isotope value for a metabolite is calculated as follows.

Different isotopes from a given analyte are identified by analyzing the full raw data for a particular mass defect within a short retention time range. For example if two ions with 1.00335 mass difference are found within 0.01 min, the ions will be considered as isotopes (difference between 13 C and 12 C is 1.00335). Similar analysis can be performed for each of the isotopes listed above. For the metabolite analysis method of the invention, the most relevant isotopes are carbon, oxygen, sulphur, nitrogen, and chlorine. Data analysis scripts to perform such an analysis are well known and can be readily utilized to perform this task. By way of example, and not to be limiting, the accompanying examples section provides details of isotope data analysis scripts which can be used in the performance of the method of the invention.

An adduct ion is formed from a full scan ion and contains all of the constituent atoms of that ion as well as additional atoms or molecules. Adduct ions are often formed in a mass spectrometer ion source. Adduct variation between full scan ions is therefore a variation in the mass of full scan ions all derived from the same metabolite. By way of example, and not to be limiting, the accompanying examples section provides details of adduct clustering data analysis scripts which can be used in the performance of the method of the invention.

Different adducts from a given analyte are identified by analyzing the full raw data for a particular mass defect within a short retention time range. Here the mass defect used for adduct recognition is dependent on the polarity of ionization (positive or negative electrospray). For example, a mass difference of 18.033823 corresponds to the adduct NH4+. Other adducts and their mass differences are well known in the art. Data analysis scripts to perform such an analysis are well known and can be readily utilized to perform this task.

From the information provided herein, it can be seen that different full scan ions can be determined to be derived from the same metabolite. Hence following the methodologies provided herein, a full data cluster set can be derived from the full raw data set so as to group different full scan ions together and assign them to a common metabolite source.

b) Generating a SWATH® Data Cluster Set from the Raw SWATH® Data Set

The method of the invention includes in step v) the generation of a SWATH® data cluster set from the raw SWATH® data set.

As outlined above, in step iv) a tandem mass spectrometry analysis of the extracted metabolites is performed with a plurality of mass selection windows to generate a raw SWATH® data set for fragment ions derived from precursor ions in a selection window. The raw SWATH® data set comprises m/z and retention time data as well as intensity values.

The raw SWATH® data set is then analyzed to assign fragment ion data to specific full scan ion data. This can be completed by linking fragment ions according to retention time: a common retention time of different fragment ions indicates that they are derived from the same precursor ion and hence the same metabolite.

However, as can be appreciated, a metabolite will elute from the chromatography column over a period of time (retention time, “RT”). Accordingly RT of peaks, resulting from the aggregation of primary data points, may have differences for the same metabolite for different extracted samples run individually on a LC system.

The differences in the RT of metabolite for a specific LC-setup, the RT variance, are composed of essentially three main factors: (i) metabolite-intrinsic properties, (ii) variance in the LC-system, and (iii) residual variance.

Metabolite intrinsic retention (i) is specific for each metabolite (chemical composition, isotope or adduct modifications) and determined by its physicochemical properties, in particular in the context of chromatography specific parameters (mobile phase composition, pH of mobile phase for LC, stationary phase), in a way that defies highly accurate prediction.

The setup of the chromatographic system (e.g. solvent gradient, and/or column material, and/or dead volumes in the LC system) can affect all metabolites in a consistent way and is called here LC-variance (ii).

The residual variance (iii) is composed of variability, such as effects of varying sample concentrations (resulting in overloading) etc.

Hence there will be a range in which the retention times of a fragment ion vary, but which nonetheless relate to the same metabolite. Therefore, the raw SWATH® data set is analyzed to cluster fragment ions according to similar retention times and so generate a SWATH® data cluster set within each SWATH® window.

Means of performing such a data clustering are well known in the art.

A preferred method of performing such an analysis is provided below.

Description: Clustering the retention time (RT) values from the measured samples at selected amounts, where different amounts of a sample can be provided by dilution of the sample at different levels and using same amounts of differently diluted samples as well. Dilutions can be prepared by weight or volume and amounts can be determined by weighing or taking volumes as well.

Statistical method: The method uses optimal k-means clustering in one dimension by dynamic programming, as implemented in the Ckmeans.1d.cp package provided in Wang, H. and Song, M. (2011) Ckmeans.1d.dp: optimal k-means clustering in one dimension by dynamic programming. The R Journal 3(2), 29-33.

This method minimizes the unweighted within-cluster sum of squared distance (L2).

In contrast to the heuristic k-means algorithms, this function optimally assigns elements to k clusters by dynamic programming. It minimizes the total of within-cluster sums of squared distances between each element and its corresponding cluster mean.

When a range is provided for the number of clusters, the exact number is determined by Bayesian information criterion. In this case, the range of potential cluster number was set to 1 to 50.

R-Package used: Ckmeans.1d.dp

R-Function: Ckmeans.1d.dp

Output: Annotated fragment ions of 1 Da precursor ion mass selection window describing fragment ion clusters.

Plots of m/z over RT of fragment ions are generated for each 1 Da precursor ion mass selection range coloring the individual fragment ions according to their assignment to a given cluster. Calculate cluster width, minimum and maximum cluster border. This step requires no additional statistical method. The limits and range of each cluster are defined, respectively, based on the minimum and maximum observed RT within that cluster. Once the minimum and maximum observations are identified, the range is defined as [min(RTobs)−LLobs, max(RTobs)+ULobs] where LLobs is the lower limit of the confidence interval for the measurement of min(RTobs), and ULobs is the upper limit of the confidence interval for the measurement of max(RTobs), as reported by the processing software for primary LC-MS/MS raw data (e.g. RefinerMS from Genedata Expressionist®). The objective is to define a range of RTs that represents every cluster, which can be used for assigning RT values from the full scan data to the clusters based on fragment ion data.

Output: A table containing cluster number, lower and upper bound and range.

Accordingly, this data analysis method provides a SWATH® data cluster set consisting of fragment ion clusters within each SWATH® window.

From the above information, it can be seen, that different full scan ions can be assigned to a cluster (in a grouping and clustering step performed independently from the fragment ion clustering step) as well and so be known to be derived from the same metabolite. In an embodiment of the invention the SWATH® data cluster set comprises mass (precursor ion mass selection window), retention time, fragment and intensity data for the fragment ions.

c) aligning the SWATH® data cluster set with the full data cluster set

The method of the invention includes in step vi) the aligning the SWATH® data cluster set with the full data cluster set.

As provided above, the full data cluster set groups different full scan ions together and assigns them to a common metabolite source. Each full scan ion is characterized by intensity, retention time data and m/z data.

Also as stated above, a SWATH® data cluster set groups fragment ions according to similar retention times and assigns them to a common precursor ion source. The SWATH® data cluster set comprises mass, retention time, fragment and intensity data for the fragment ions.

In this step of the method of the invention, the retention time and mass of the SWATH® data cluster set is aligned to full data cluster set according to common characteristics. In practice, where a SWATH® data cluster has a certain retention time in common with a full data cluster, then the SWATH® data cluster is aligned with that full data cluster. This means that the SWATH® data cluster is aligned to a specific precursor ion in the full data cluster.

The alignment of the SWATH® data cluster set with the full data cluster set can be performed according to the following methodology.

Description: The assumption is that peaks with similar masses and similar retention times should come from measuring the same analytes. In this step we relate the measured retention times in the full data cluster set to the SWATH® data cluster set.

The first step is to link peaks of the full data cluster set to clusters of the SWATH® data cluster set through the mass windows. Full scan measurements (peaks) with an ion mass (m/z) within a mass window M will be linked to fragment ion clusters originating from a precursor ion mass selection window (SWATH® windows) of the same m/z range Once they align on a given mass window, we focus on the retention times and assign peaks to a cluster, if they have retention time values that fall within the range of that given cluster. This peak becomes an annotation indicating to which cluster from which mass window it belongs.

This step requires no statistical analysis.

Output: Files describing which full data cluster set belongs to which SWATH® data cluster set.

At the end of this process, it is possible to determine the following data as being derived from the same metabolite. First, the full data cluster set includes for each metabolite: the mass, retention time, isotope and adduct values and intensities of full scan ions. Also, the SWATH® data cluster set includes for each metabolite mass, retention time, fragment and intensity data for the fragment ions.

Hence in an embodiment of the invention this step in the method provides a characteristic profile for each extracted metabolite which comprises (a) mass, retention time, isotope, adduct values and intensity of full scan ions, and (b) mass, retention time, fragment and intensity data for the fragment ions.

d) comparing the characteristic profile of each extracted metabolite obtained in step vi) with a reference library of characteristic profiles of metabolites to provide the metabolic content of the biological sample

The method of the invention includes in step vii) comparing the characteristic profile obtained in step vi) of each extracted metabolite with a reference library of characteristic profiles of metabolites. This provides the metabolic content of the biological sample.

“Characteristic profile” as used herein encompasses features which characterize the physical and/or chemical properties of a metabolite. Values for said properties may serve as characteristic profile and can be determined by techniques well known in the art. Most preferably, a characteristic feature to be determined in accordance with the present invention is (a) mass, retention time, isotope and adduct values and intensity of full scan ions, and (b) mass, retention time, fragment and intensity data for the fragment ions.

The analysis used in step vii) is essentially an exercise in data mining.

As described above, the characteristic profile of each extracted metabolite comprises (a) mass, retention time, isotope and adduct values and intensity of full scan ions, and (b) mass, retention time, fragment and intensity data for the fragment ions.

The data analysis comprises the use of fragment ion characteristic profile data and data mining of reference spectra libraries of characteristic profiles of metabolites. Reference spectra libraries of characteristic profiles of metabolites may be generated for pools of synthetic metabolites and/or from prior extensive MS metabolism analyses performed on the biological sample under investigation. Similarly, the reference spectra libraries of characteristic profiles of metabolites may be generated from synthetic metabolites references and/or from prior analyses of metabolites. Importantly, once the spectra libraries of characteristic profiles of metabolites have been generated they can be used perpetually.

Hence in an embodiment of the invention the reference library of characteristic profiles of metabolites of step vii) comprises predetermined characteristic profiles of predetermined metabolites. In a further embodiment of the method of the invention the predetermined characteristic profiles of predetermined metabolites are determined from authentic standards of the known compounds, from an analysis of samples containing the compounds, from existing spectral libraries, or computationally generated by applying empirical or a priori fragmentation or modification rules to the known compounds.

In a further embodiment of the method of the invention the predetermined characteristic profiles of predetermined metabolites are used to assign the characteristic profile of each extracted metabolite to a predetermined metabolite.

The confidence in the metabolites identification can be scored, for example, based on the mass accuracy and/or the relative intensities of the acquired fragment ion fragments compared to that of the reference (or predicted) fragmentation spectrum, on the number of matched fragments, on the similar chromatographic characteristics (co-elution, peak shape, etc.) of the extracted ion traces of these fragments. Probabilities for the identifications can be determined, for example, by searching (and scoring) similarly for decoy full scan ion and/or fragment ions from the same LC-MS dataset. The relative quantification can be performed by integration of the fragment ions traces across the chromatographic elution of the related full scan ions (precursors aligned to fragments). In various embodiments, use is made of differently isotopically labeled reference analytes (similarly identified, quantified and scored) to achieve absolute quantification of the corresponding full scan ions of interest.

Hence an embodiment of the invention is wherein step vii) comprises calculating a score that represents how well the predetermined characteristic profile of predetermined metabolites and characteristic profile of each extracted metabolite match.

Metabolite annotation is performed by comparing the m/z from each ion (ion full scan as well as in MS/MS) contained in the library and the retention time of the analyte. When the mass measured is within the expected range of the user (e.g. <5 ppm deviation compared to the library) and the retention time measured is within the expected range e.g. +/−0.1 min) then the ion is annotated as a match to the ion contained in the library.

The annotation of several ions provides independent indications that a given metabolite is present in the matrix. Based on the groups defined according to the steps described above, the identification of a given metabolite allows for the annotation of ions that are not included in the library when the latter have been identified as isotopes or adducts of a library compound. Thus, unknown metabolites (metabolites that are not included in the library) can be readily detected.

Using the strategies outlined above, and other alternatives which are known to the skilled person, the method provides the metabolic content of the biological sample

Method of the Invention Performed on a Plurality of Samples

An embodiment of the method of the invention is wherein a plurality of samples of extracted metabolites from the biological sample are analyzed. A preferred embodiment of the method of the invention is wherein the samples of extracted metabolites are derived from different amounts of biological sample.

In this embodiment of the invention, the method is repeated using different samples of extracted metabolites. The benefit of repeating the method is that multiple independent performances can increase likelihood of identifying metabolites.

One embodiment is wherein the samples are derived from the same amount of biological samples. However, a preferred embodiment is wherein differing amounts of the biological sample are used to derive the extracted metabolites.

Correlations between the pluralities of amounts metabolites may be determined using any suitable algorithm or method. Examples include the Pearson correlation (after an appropriate transformation to achieve normality), Spearman p correlation, Kendall's τ correlation, and Somer's D correlations, as well as other widely-accepted standard definition employing least-squares curve fitting.

In essence, ideally the amount of a metabolite in a sample should be positively correlated with amount of the biological sample used to extract the metabolites: higher amounts of biological sample should provide higher amounts of metabolite. This relationship can be statistically measured using a simple linear regression model (SLRM) analysis.

As is known in the art, in simple linear regression, scores are predicted scores on one from the scores on a second variable. The variable to be predicted is called the criterion variable and is referred to as Y. The variable on which predictions are based is called the predictor variable and is referred to as X. When there is only one predictor variable, the prediction method is called simple regression. In simple linear regression, the predictions of Y when plotted as a function of X form a straight line.

An example of a SLRM which can be used is provided in the accompanying Examples and provided below.

Assessing the Linearity of the Measured Peak Areas in Relation to the Dilution of the Samples

Description: Simple linear regression models (SLRMs) (Chambers, J. M. (1992) Linear models.

Chapter 4 of Statistical Models in S. ed J. M. Chambers and T. J. Hastie, Wadsworth & Brooks/Cole; Wilkinson, G. N. and Rogers, C. E. (1973) Symbolic descriptions of factorial models for analysis of variance. Applied Statistics, 22, 392-9.) analysis of the peak areas with the dilution percentages as independent variables, where dilution percentage (also stated as dilution or dilution level) represents the sample matrix amount in a diluted sample.

The advantage of fitting SLRM to the data instead of just calculating a correlation coefficient is that we can also extract the slope of the fitted line. Measurements with negative slopes or correlation coefficients that are too low (preferably <0.7), might point to analytical problems. Such cases might be filtered out from the dataset if desired.

Non linear relationship have not been implemented yet but can improve the strategy in order to find ion species that are slightly above limit of detection or ion species that are in the saturation of the detector at the higher levels of the correlation curve.

Statistical Methods: Simple linear regression model (SLRM) for the peak values as response variable and the known dilution of the samples as unique explanatory variable, together with an intercept term.

peak value˜a+b*dilutions Formula:

The r², called coefficient of determination that is calculated when fitting these models, is also the square of the sample Pearson correlation coefficient r (Karl Pearson (20 Jun. 1895) “Notes on regression and inheritance in the case of two parents,” Proceedings of the Royal Society of London, 58: 240-24 which measures the linear correlation between two variables X and Y. The Pearson correlation coefficient has values between +1 and −1, where 1 is total positive linear correlation, 0 is no linear correlation, and −1 is total negative linear correlation.

The models were fitted using the lm function from the stats package in the R Language and Environment for Statistical Computing (R Core Team (2016). R: A language and environment for statistical computing. R Foundation for Statistical Computing, Vienna, Austria. URL https://www.Rproject.org/.)

### REFERENCES

Chambers, J. M. (1992) Linear models. Chapter 4 of Statistical Models in S eds J. M. Chambers and T. J. Hastie, Wadsworth & Brooks/Cole.
Wilkinson, G. N. and Rogers, C. E. (1973) Symbolic descriptions of factorial models for analysis of variance. Applied Statistics, 22, 392-9.

R-function: lm( ) Information provided in the references cited above.

Output: Full scan and SWATH® (1 Da precursor ion mass selection window) fragment ions annotated with the following values: Pearson correlation coefficient for peak vs dilution; Slope of the fitted regression line for peak vs dilution; Histograms describing the distribution of the correlation coefficients.

SLRM allows a value to be given to a metabolite according to the correlation between the amount of the metabolite measured and the amount of the biological sample used to extract the metabolites. As used herein, this is termed the “SLRM value”.

Applications of the Method of the Invention

As stated above, the method of the invention allows for the identification of a semi-quantitative analysis of all detectable metabolites present in a biological sample which clearly provides an important resource for determining the metabolome of that sample.

The approach includes any biotechnical, biomedical, pharmaceutical and biological applications that rely on qualitative and quantitative LC-MS analysis. The approaches are, for example, in various embodiments particularly suited to perform the analysis of biological samples having a high number of metabolites of interest in complex samples that may be available only in limited amounts (e.g., complete organisms, cells, organs, bodily fluids, etc.). The approach is applicable to the analysis of proteins from all organisms, from cells, organs, body fluids, and in the context of in vivo and/or in vitro analyses. Examples of applications of the invention include the development, use and commercialization of quantitative assays for sets of polypeptides of interest.

The invention can be beneficial for the pharmaceutical industry (e.g. drug development and assessment), the biotechnology industry (e.g. assay design and development and quality control), and in clinical applications (e.g. identification of biomarkers of disease and quantitative analysis for diagnostic, prognostic and/or therapeutic use). The invention can also be applied to water, drink, food and food ingredient testing, for example, quantifying nutrients, contaminants, toxins, antibiotics, steroids, hormones, pathogens, and allergens in water, drinks, foods and food ingredients.

A method of identifying a metabolic signature of a biological sample

One such utility of the method is therefore to identify a metabolic signature of a biological sample.

This can be considered to be a subset of the population of identified metabolites which can be identified and routinely applied to other samples in a reproducible and automated manner.

Hence a further aspect of the method of the invention provides a method of identifying a metabolic signature of a biological sample comprising
i) providing two or more samples of extracted metabolites derived from different amounts of the biological sample;
ii) performing a chromatography coupled mass spectrometry analysis of the extracted metabolites to generate a full raw data set for full scan ions;
iii) generating a full data cluster set from the full raw data set obtained in step ii) by grouping full scan ions according to isotope and adduct values;
iv) performing a tandem mass spectrometry analysis of the extracted metabolites with a plurality of mass selection windows to generate a raw SWATH® data set for fragment ions;
v) generating a SWATH® data cluster set from the raw SWATH® data set obtained in step
iv) by grouping fragment ions according to retention time and mass values;
vi) aligning the SWATH® data cluster set with the full data cluster set to generate characteristic profile for each extracted metabolite;
vii) comparing the characteristic profile of each extracted metabolite obtained in step vi) with a reference library of characteristic profiles of metabolites to provide the metabolic content of the biological sample;
viii) performing a simple linear regression model (SLRM) analysis for the raw data set and SWATH® data cluster set to generate a SLRM value for the metabolite;
ix) selecting those metabolites which have a SLRM correlation coefficient of at least 0.7 as the signature.

A High-Throughput Screening Method of Analyzing Metabolites in a Biological Sample

One such utility of the method is high-throughput screening method of analyzing metabolites in a biological sample. This can use the metabolic signature of a biological sample as discussed above.

Hence a further aspect of the method of the invention provides a high-throughput screening method of analyzing metabolites in a biological sample comprising performing the method of the invention, and analyzing the signature metabolites in the biological sample.

A further aspect of the method of the invention is to use the metabolic signature of a biological sample (RT, precursor and fragment ion m/z) for a fast multi-target most selective and sensitive high-throughput screening method of analyzing metabolites in a biological sample in one analysis per sample in less than 15 min. using a LC-MS/MS method on a Triple Quadruple Mass Spectrometer.

Sample Preparation

The term “providing” as used herein means that the at least one biological sample is provided in a manner suitable for determining the metabolic content comprised by said biological sample. Accordingly, providing as used herein also refers to carrying out suitable pre-treatments, i.e. most preferably concentration or fractioning of the sample and/or extraction of the sample. Depending on the technique which is used to determine the at metabolic content comprised by said biological sample, additional pre-treatments may be required.

The sample is prepared in the following way. A mixture of methanol, water and dichloromethane is added to the biological sample, preferably a plasma sample. The resulting mixture is shaken several minutes and centrifuged, using standard laboratory methods. An aliquot of the resulting liquid extract is taken for further analysis, which is termed “extracted metabolites”.

Biological Sample

The term “biological sample”, as used herein, relates to a sample comprising a biological material, wherein the term “biological material”, preferably, includes any substance or mixture of substances produced by a cell, preferably including substances and mixtures of substances produced by such biological material. Preferably, the biological material comprises a multitude of metabolites of a cell. As used herein, the term “multitude of metabolites” preferably relates to at least 50, more preferably at least 100, even more preferably at least 200, most preferably at least 300 metabolites of a cell. Preferably, the biological sample is a sample of a material comprising a non-defined mixture of compounds, such as a cell culture medium comprising serum, a spent cell culture medium, a bodily fluid of an organism, tissue of an organism, and the like. Thus, preferably, the biological sample is a cell culture sample from archaebacterial, bacterial, and/or eukaryotic cells, wherein said cell culture sample preferably comprises cells and/or spent culture medium; preferably, in such case, the biological sample is a sample of cultured bacterial, fungal, plant, such as a dicot or monocot plant, more preferably a crop plant., algae, human or animal cells and/or spent medium of said cells. Most preferably, the biological sample is a sample of and/or spent culture medium from E. coli cells, Paenibacillus cells, Basfia succiniciproducens cells, Corynebacterium glutamicum, Lactobacillus, Bacillus acidopullulyticus cells, Bacillus amyloliquefaciens cells, Bacillus lentus cells, Bacillus licheniformis cells, Bacillus subtilis cells, Aspergillus niger cells, Aspergillus oryzae cells, Chrysosporium lucknowense cells, Myceliophthora thermophile cells, Penicillium chrysogenum cells, Penicillium funiculosum cells, Rhizomucor miehei cells, Schizophyllum commune cells, Trichoderma harzianum cells, Trichoderma longibrachiatum cells, Trichoderma reesei cells, yeast cells, Saccharomyces cerevisiae cells, Schizosaccharomyces pombe cells, Pichia pastoris cells, Kluyveromyces lactis cells, Kluyveromyces fragilis cells, Candida rugose cells, Candida lipolytica cells, Candida Antarctica cells, CHO cells (Chinese hamster ovary cells), liver cells, hepatocytes, kidney cells, kidney cancer cells, pancreatic cells, pancreatic cancer cells, cardiac cells, cardiac cancer cells, endothelial cells, endothelial cancer cells, fibroblasts, lung cells, lung cancer cells, bladder cells, bladder cancer cells, breast cells, breast cancer cells, colon cells, colon cancer cells, ovarian cells, ovarian cancer cells, duodenum cells, duodenum cancer cells, bile duct cells, bilde duct cancer cells, stem cells or skin cells.

As used herein, the term “plant” relates to a whole plant, a plant part, a plant organ, a plant tissue, or a plant cell. Thus, the term includes, preferably, seeds, shoots, stems, leaves, roots (including tubers), and flowers. Preferably, the term “plant” relates to a member of the clade Archaeplastida. Plants that are particularly useful in the methods of the invention include all plants which belong to the superfamily Viridiplantae, preferably Tracheophyta, more preferably Spermatophytina, most preferably monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Moms nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others. Preferably, the plant cell, plant or plant part is a rice cell, rice plant, rice plant part, or rice seed.

Preferably, the sample is a sample from a multicellular organism. More preferably, the sample comprises a bodily fluid of an organism and/or a tissue of an organism. Preferably, the biological sample is a sample of an animal, preferably a vertebrate, more preferably a mammal. More preferably, the biological sample is a sample of an egg, a, preferably non-human, embryo, or a complete non-human organism, e.g. an insect, a nematode, or a laboratory animal. Preferably, the biological sample is or comprises a sample of a body fluid, a sample from a tissue or an organ, or a sample of wash/rinse fluid or a swab or smear obtained from an outer or inner body surface. Preferably, samples of stool, urine, saliva, sputum, tears, cerebrospinal fluid, blood, serum, plasma, lymph or lacrimal fluid are encompassed as biological samples by the method of the present invention. In particular in multicellular organisms, biological samples can be obtained by use of brushes, (cotton) swabs, spatula, rinse/wash fluids, punch biopsy devices, puncture of cavities with needles or lancets, or by surgical instrumentation. However, biological samples obtained by well known techniques including, in an embodiment, scrapes, swabs or biopsies are also included as samples of the present invention. Cell-free fluids may be obtained from the body fluids or the tissues or organs by lysing techniques such as homogenization and/or by separating techniques such as filtration or centrifugation. It is to be understood that a sample may be further processed in order to carry out the method of the present invention. Particularly, cells may be removed from the sample by methods and means known in the art. More preferably, the biological sample is a sample of a body fluid, preferably a blood, plasma, or serum sample. Also more preferably, the biological sample is a tissue sample, preferably a sample of liver tissue, heart tissue, prostate tissue, pancreas tissue, brain tissue, kidney tissue, adipose tissue, gut, skeleton tissue, lung tissue, bladder, breast tissue, cecum and/or skin tissue, such as dermal layer, comprising the epidermis and/or corium and/or subcutis. Also preferably, the biological sample is a sample of an algae or plant, preferably of a monocotyledonous or dicotyledonous plant. More preferably, said biological sample is a tissue sample, preferably leaf tissue, root tissue, shoot tissue, stem tissue, reproductive tissue (such as flower tissue or pollen) and/or seed tissue and/or liquid comprising exudate thereof and/or volatile compounds released thereof.

Metabolites

The term “metabolite”, as used herein, relates to at least one molecule of a specific metabolite up to a plurality of molecules of the said specific metabolite. It is to be understood further that a group of metabolites means a plurality of chemically different molecules wherein for each metabolite at least one molecule up to a plurality of molecules may be present. A metabolite in accordance with the present invention encompasses all classes of organic or inorganic chemical compounds including those being comprised by biological material such as animals or plants. Preferably, a metabolite has a molecular weight of from 25 Da (Dalton) to 300,000 Da, more preferably of from 30 Da to 30,000 Da, most preferably of from 50 Da to 1500 Da. Preferably a metabolite has a molecular weight of less than 30,000 Da, less than 20,000 Da, less than 15,000 Da, less than 10,000 Da, less than 8,000 Da, less than 7,000 Da, less than 6,000 Da, less than 5,000 Da, less than 4,000 Da, less than 3,000 Da, less than 2,000 Da, less than 1,500 Da, less than 1,000 Da, less than 500 Da, less than 300 Da, less than 200 Da, or less than 100 Da. Preferably, a metabolite has, however, a molecular weight of at least 50 Da.

Preferably, the metabolite is a biological macromolecule, e.g. preferably, DNA, RNA, protein, or a fragment thereof, e.g., preferably a fragment produced by processing of sample material. More preferably, in case a plurality of metabolites is envisaged, said plurality of metabolites is representing a metabolome, i.e. the collection of metabolites being comprised by an organism, an organ, a tissue, a body fluid, a cell or a part of a cell at a specific time and under specific conditions.

More preferably, the metabolite in accordance with the present invention is a small molecule compound, such as a substrate for an enzyme of a metabolic pathway, an intermediate of such a pathway or a product obtained by a metabolic pathway. Metabolic pathways are well known in the art and may vary between species. Preferably, said pathways include at least citric acid cycle, respiratory chain, photo respiratory chain, glycolysis (Embden-Meyerhof-Parnas (EMP) pathway), gluconeogenesis, hexose monophosphate pathway, starch metabolism, oxidative and non oxidative pentose phosphate pathway (Calvin-Benson (CB) cycle, glyoxylate metabolism, production and β-oxidation of fatty acids, urea cycle, amino acid biosynthesis pathways, protein degradation pathways such as proteasomal degradation, amino acid degrading pathways, biosynthesis or degradation of lipids, polyketides (including e.g. flavonoids and isoflavonoids), isoprenoids (including eg. terpenes, sterols, steroids, carotenoids, xanthophylls), carbohydrates, phenylpropanoids and derivatives, alcaloids, benzenoids, indoles, indole-sulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs. Accordingly, small molecule compound metabolites are preferably composed of the following classes of compounds: alcohols, alkanes, alkenes, alkines, aromatic compounds, ketones, aldehydes, carboxylic acids, esters, amines, imines, amides, cyanides, amino acids, peptides, thiols, thioesters, phosphate esters, sulfate esters, thioethers, sulfoxides, ethers, or combinations or derivatives of the aforementioned compounds. The small molecules among the metabolites may be primary metabolites which are required for normal cellular function, organ function or animal or plant growth, development or health. Moreover, small molecule metabolites further comprise secondary metabolites having essential ecological function, e.g. metabolites which allow an organism to adapt to its environment. Furthermore, metabolites are not limited to said primary and secondary metabolites and further encompass artificial small molecule compounds. Said artificial small molecule compounds are derived from exogenously provided small molecules which are administered or taken up by an organism but are not primary or secondary metabolites as defined above, including, preferably, drugs, herbicides, fungicides, and insecticides. Moreover, artificial small molecule compounds may be metabolic products of compounds taken up, and preferably metabolized, by metabolic pathways of an organism. Moreover, small molecule compounds preferably include compounds produced by organisms living in, on or in close vicinity to an organism, more preferably by an infectious agent as specified elsewhere herein, by a parasitic and/or by a symbiotic organism.

### Example 1: General Steps of Data Generation and Data Analysis

Introduction

The method of the invention allows for the semi-quantitative analysis of the metabolites present in a biological sample using Liquid Chromatography-Mass Spectrometry (LC-MS)-based instrumentation. Semi-quantitative analysis, in this context, represents the generation of ratio values from amounts. concentrations, intensities or quantities of identical ion species, representing and referring to the identical metabolites, of different samples, analyzed in the same experiment, with no need for calibration with reference standards.

The first step in the procedure is designed to obtain an accurate inventory of small molecules in the sample matrix. To this end, liquid chromatography coupled to high resolution Q-TOF-MS (Quadrupole-Time of Flight Mass Spectrometry) is applied, which provides accurate mass data to facilitate metabolite identification and full-scan information to enable the detection of as many metabolites as possible in an untargeted way. Untargeted analysis, in this context, stands for an analysis, which is open for providing result values for metabolites contained in a sample, which are not known in total, and no individual analysis parameters are needed being specified and set before analysis for individual analytes referring to those metabolites, but the analysis provides not only result values, e.g. intensity values, but also information describing and distinguishing individual ion species related to those metabolites (by e.g. RT, m/z values and spectra). In this method the set of those ion species represent the inventory of small molecules in the sample matrix. This step can be repeated with different chromatography systems (HILIC, reversed phase, different mobile phases) to achieve most complete inventory coverage of small molecules in a sample matrix.

Secondly, triple quadrupole (QqQ) LC-MS is much more suitable for the actual high-throughput metabolite profiling measurements, due to its robustness and ability to quantify metabolites with concentrations across several orders of magnitude. In addition to LC analysis, each biological extract may be analyzed with GC. The workflow for the method of the invention is provided in FIG. 1.

Data Acquisition and Analysis

1. Experimental design and metabolite extraction

Extracts are prepared from the same biological sample or from a pool of samples to ensure that the chemical composition of each extract is identical.

Five distinct amounts are extracted in order to obtain five different sample strengths or dilutions (extraction procedure provided in example 2). In addition, a procedural blank is also prepared. Single extracts are prepared for all levels except for the “median level” which is prepared in triplicate.

2. Untargeted Q-ToF Analysis and data generation

A typical method for a certain sample matrix (e.g. plant, blood plasma, urine, cells or tissue material) proceeds with a statistical analysis of the recorded spectral data. Here, a list of all features that can be detected with a certain statistical significance in the actual sample material is obtained. In this context, features are unambiguously defined by their chromatographic and mass spectrometric parameters: exact mass, retention time, the masses of fragments and the intensities of these fragments.

Subsequently, an annotation procedure is employed to assign metabolite identities to all recorded features by matching the data to a library. This library contains spectral and RT information of commercially acquired metabolite standards, as well as previously identified metabolites from real tissue material for which standards are not available. The result is a list of all metabolites in the specific matrix, which is used to generate the MRM transitions that constitute the high-throughput QqQ method.

Untargeted analysis is performed using a quadrupole time of flight (QToF) mass spectrometer. The preferred method consisted in generating MS/MS of all masses from 100 to 1000 Da (SWATH® at unit resolution, 1 Da) and full scan but other approaches are possible.
I. MS/MS of all features (SWATH® at unit resolution, 1 Da) and full scan
II. SWATH® with fixed mass selection window (25 Da) and full scan
III. SWATH® with variable mass selection window and full scan
IV. Full Scan (no MS/MS)

I. Fragment Ion Scan (MS/MS) of all features (SWATH® at unit resolution, 1 Da)

This approach has the advantage that MS/MS spectra are generated from unit resolution (1 Da) instead of 25 Da in a SWATH® experiment. The current performances of Q-ToF instrumentation allow for 22 to 23 fragment ion scans to be carried out simultaneously, meaning that per sample run, 22 or 23 consecutive precursor masses are individually selected and all resulting fragment ions recorded. Therefore, 40 injections per sample would be required to cover the entire range of interest 100-1000 Da (40×22.5 Da=900 Da). The number of MS/MS per sample run is depending on the smallest peak width in the sample chromatogram. Subsequently, the fragment ion spectra are matched against the library spectra to facilitate the identification of metabolites. The major advantage of this procedure is that interfering spectra from co-eluting substances can be minimized which greatly decreases the number of false negative results.

Peak finding and peak integration were performed using Genedata Expressionist® 10.2 Refiner MS module in a batch process mode. Each 1 Da window was handled independently i.e. peak alignment and noise reduction were performed within each 1 Da window. The automated Genedata Refiner MS workflow comprises the following steps: loading of spectral data, gridding, data preprocessing (background subtraction, noise removal, intensity thresholding), retention time alignment, quantification (peak detection, isotope clustering, peak annotation), export of data. Peaks were annotated based on retention times, precursor window, and accurate mass of the quantitation fragment based on an in-house library where retention times, survey scan exact masses and fragment ion scans were obtained from commercial standards or from plant extracts when analytes of interest are unknown or not available. FIG. 2 shows how the data is generated and analyzed according to the method of the invention.

II. SWATH® Technology with fixed mass selection window (25 Da)

Sequential Window Acquisition of all Theoretical fragments (SWATH®) can be employed to carry out metabolite profiling. The principle of the SWATH® acquisition relies on simultaneous conduction of the following steps:
1. A survey scan with low collision energy covering the user-defined mass range (here 100-1000 Da) i.e. Q1 set to full transmission (MS only)
2. The mass range is then scanned using predefined Q1 windows (here 25 Da) applying a range of collision energies (rolling collision energy, i.e. increasing collision energy with analyte mass) to produce product ion spectra from each mass range (MS/MS)

The identification of the metabolites is based on an in-house library where retention times, survey scan exact masses and product ion scans were obtained from commercial standards or from plant extracts when analytes of interest are unknown or not available. MS/MS spectra for the library were acquired by a classical fragment ion scan.

One selective fragment ion can then be selected as quantitation ion and used to generate extracted ion chromatogram (XIC) from the SWATH® window corresponding to the precursor ion.

Peak finding and peak integration were performed using Genedata Expressionist® 10.2 Refiner MS module in a batch process mode. Each SWATH® window was handled independently i.e. peak alignment and noise reduction were performed within each SWATH® window. Peaks were then annotated based on retention times, SWATH® window, and accurate mass of the quantitation fragment. The automated Genedata Refiner MS workflow comprises the following steps: loading of spectral data, gridding, data preprocessing (background subtraction, noise removal, intensity thresholding), retention time alignment, quantification (peak detection, isotope clustering, peak annotation), export of data matrix.

The advantage of the all workflows described relies on the capability of processing the data of several replicates at the same time before performing the identification against the library.

Thus, small unwanted variability in the retention times of analytes of interest or in the accuracy of determination of the exact masses are corrected. The statistical power obtained with such method is significantly higher than attempting to identify metabolites from one single sample. As a result, the identification of metabolites in a given matrix can be performed with higher confidence (limit false negative or false positive).

III. SWATH® with variable mass selection window

The principle of SWATH® with variable windows is identical to the conventional SWATH®. The difference is that, instead of using a fixed Q1 window (25 Da in the examples above), the Q1 window will vary according to mass range of interest. An example is given below
100-150: 5 Da increments->10 windows
150-250: 10 Da increments->10 windows
250-500: 25 Da increments->10 windows
500-1000:100 Da increments->5 windows

IV. Full Scan

Full scan high resolution acquisition are also tested for the matrix characterization. Identification of metabolites based on full scan data only relies on the exact mass of the analyte as well as the retention times. The selectivity of the acquisition is therefore lower than described above and no advantage of MS/MS spectral libraries can be taken to identify compounds.

It has to be mentioned that running the SWATH® method as an untargeted procedure, meaning that all spectral data are captured, has the disadvantage that data variability is slightly higher and sensitivity and linear range is lower than for the preferred Multi-MRM method.

3. Clustering of Data Using R

Data: RT and Mass values from a number of samples with different dilutions (e.g. 0, 10, 20, 30, 40 mg of sample per constant extraction solvent volume)

Programming language: R, A language and environment for statistical computing.

Step 1. Data Pre-Processing

Description: Gather all individual mass files into a single file to be statistically analyzed (for fragment ion data, full scan data is treated separately).

Step 2. Assessing the Linearity of the Measured Peak Areas in Relation to the Dilution of the Samples

Description: Ideally, the amount of a given substance in a sample should be positively correlated with the concentration of that same sample in the measured dilution (higher concentrations should lead to higher measurements of a given substance/analyte), and therefore negatively correlated with the dilution level of the sample. Given that we measured 5 dilution levels for each mother sample, it is possible for us to statistically estimate this correlation. (FIG. 3).

We decided to fit simple linear regression models (SLRMs) [Ref.1, Ref 2.] to the peak areas, with the dilution percentages as independent variables, where dilution percentage (also stated as dilution or dilution level) represents the sample matrix amount in a diluted sample.

The advantage of fitting SLRM to the data instead of just calculating a correlation coefficient is that we can also extract the slope of the fitted line. Measurements with negative slopes, slopes close to zero, or correlation coefficients that are too low (preferably <0.7), might point to analytical problems. Such cases might be filtered out from the dataset if desired.

Non linear relationship have not been implemented yet but can improve the strategy in order to find valid ion species that are slightly above limit of detection or features that are in the saturation of the detector at the higher levels of the correlation curve.

Statistical Methods:

Simple linear regression model (SLRM) [Ref.1, Ref 2.] for the peak values as response variable and the known dilution of the samples as unique explanatory variable, together with an intercept term.

peak value˜a+b*dilutions Formula:

The r², called coefficient of determination that is calculated when fitting these models, is also the square of the sample Pearson correlation coefficient r [Ref. 3] which measures the linear correlation between two variables X and Y. The Pearson correlation coefficient has values between +1 and −1, where 1 is total positive linear correlation, 0 is no linear correlation, and −1 is total negative linear correlation.

The models were fitted using the lm function from the stats package in the R Language and Environment for Statistical Computing [Ref. 4].

R-function: lm( ) [Ref. 5]

Output:

Full scan and fragment ion data annotated with the following values:
Pearson correlation coefficient for peak vs dilution
Slope of the fitted regression line for peak vs dilution

Histograms describing the distribution of the correlation coefficients.

Step 3. Clustering Retention Time (RT) Values

Description: Clustering the retention time (RT) values from the measured samples at selected dilution levels.

Statistical Method:

Optimal k-means clustering in one dimension by dynamic programming, as implemented in the Ckmeans.1d.cp package [Ref.6] and function in R.

This method minimizes the unweighted within-cluster sum of squared distance (L2).

In contrast to the heuristic k-means algorithms, this function optimally assigns elements to k clusters by dynamic programming [Ref. 6]. It minimizes the total of within-cluster sums of squared distances between each element and its corresponding cluster mean.

When a range is provided for the number of clusters, the exact number is determined by Bayesian information criterion. In this case, the range of potential cluster number was set to 1 to 50.

R-Package: Ckmeans.1d.dp

R-Function: Ckmeans.1d.dp

Output: Annotated fragment ion file describing which fragment ion belongs to which cluster.

Plots are generated for each precursor ion mass range coloring the individual fragment ions according to their assignment to a given cluster.
a) Calculate cluster width, minimum and maximum cluster border. This step requires no additional statistical method. The limits and range of each cluster are defined, respectively, as the minimum and maximum RT and the range within that cluster.
b) The objective is to define a range of RTs that represents every cluster, which can be used for assigning RT values from the full scan data to the clusters based on fragment ion mass data.

Output: A table containing cluster number, lower and upper bound and range.

Step 4. Relating full scan ions to the RT clusters identified on the fragment ion mass data

Description: The assumption is that peaks with similar masses and similar retention times should come from measuring the same analytes. In this step we relate the measured retention times in the full scan data to the clusters found on fragment ion mass data.

The first step is to link the full scan data to the fragment ion mass data through the mass windows. Full scan measurements with a precursor ion selection mass window M will be linked to fragment ion clusters identified in that same mass window. Once they align on a given mass window, we focus on the retention times and assign peaks to a cluster, if they have RT values that fall within the range of that given cluster. This peak becomes an annotation indicating to which cluster from which mass window it belongs.

This step requires no statistical analysis.

Output: Annotated full scan data file describing which full scan ion belongs to which fragment ion mass data cluster.

Step 5. Annotate the Fragment Ion Mass Data with the Description and Group from the Full Scan Data

Description: The idea here is that, by linking the full scan and the fragment ion mass data by mass windows and corresponding clusters, we have identified analytes/peaks that belong together. Therefore, the fragment ion mass data should get the same description and the same group as the data from the full scan. This step helps in the identification of fragments that belong to the same analyte.

### REFERENCES FOR EXAMPLE 1 (NUMBERING FOLLOWING THAT ABOVE)

1. The statistical approach we selected was to fit a simple linear regression model (SLRM) Reference: Chambers, J. M. (1992) Linear models. Chapter 4 of Statistical Models in S. ed J. M. Chambers and T. J. Hastie, Wadsworth & Brooks/Cole.
2. Wilkinson, G. N. and Rogers, C. E. (1973) Symbolic descriptions of factorial models for analysis of variance. Applied Statistics, 22, 392-9.
3. Karl Pearson (20 Jun. 1895) “Notes on regression and inheritance in the case of two parents,” Proceedings of the Royal Society of London, 58: 240-242.
4. R Core Team (2016). R: A language and environment for statistical computing. R Foundation for Statistical Computing, Vienna, Austria. URL https://www.R-project.org/.
5. http://127.0.0.1:11877/library/stats/html/lm.html.
6. Wang, H. and Song, M. (2011) Ckmeans.1d.dp: optimal k-means clustering in one dimension by dynamic programming. The R Journal 3(2), 29-33.

4. Generation of a List of Potential Metabolites of Interest
Assess quality of groups using results of correlation of signals and amounts of matrix as well as variability from the replicate at the target concentration
Confirm the grouping of known metabolites present in library by number of annotated peaks (full scan and MS/MS)
Explore non annotated features groups and include in library if confirmed as new unknown compound

5. High Throughput Method

Multiple reaction monitoring (MRM), also called selected reaction monitoring (SRM), is a technology used for reliable quantification of analytes of low abundance in complex mixtures. In an MRM experiment, a predefined precursor ion and one of its fragments (products) are selected by the two mass filters of a triple quadrupole instrument and monitored over time for precise quantification.

The first and the third quadrupoles act as filters to specifically select predefined m/z values corresponding to the molecular ion and a specific fragment ion of the compound, whereas the second quadrupole serves as collision cell. Several such transitions (precursor/product ion pairs) are monitored over time, yielding a set of chromatographic traces. The two levels of mass selection with narrow mass windows result in a high selectivity, as co-eluting background ions are filtered out very effectively. Moreover, unlike in other MS-based techniques, no full mass spectra are recorded in MRM analysis. The nature of this mode of operation translates into an increased sensitivity compared with conventional full scan techniques and in a linear response over a wide dynamic range thus enabling the detection of low-abundance compounds in highly complex mixtures.

In short, MRM selects the characteristic precursor ion of the metabolite in the first quadrupole, the selected on is collided in the second quadrupole to produce the fragment ions, and the third quadrupole selects the characteristic product ion. For the QqQ Multi-MRM method, hundreds of these ion pairs, each of which is characteristic for a specific metabolite, are analyzed in a single run with high selectivity, sensitivity and a wide dynamic range. The increasing scan speeds of MS instruments have enabled the development of such large-scale MRM assays. MRM settings/conditions can be determined experimentally or derived from the QToF fragment ion scan acquisition described above. The validated list of MRM transitions then constitutes the final method.

### Example 2: Specific Example of Workflow for Data Generation and Analysis Using the Method of the Invention

Introduction

This example demonstrates that the method of the invention can be successfully applied to a set of metabolomics data to assign ion species to analytes, representing known or unknown metabolites. This method allows for the annotation of hundreds of ion species (full scan, precursor and fragment ions) and thus offers the user to focus structure elucidation efforts on real unknowns rather than of different ionized forms of known substances.

The following example shows data for one metabolite: TAG (C16:0;C18:1;C18:3). It is anticipated that the method would be equally efficient for several hundreds of metabolites simultaneously.

Material

20, 40, 60, 80 and 100 uL of rat plasma were used for analysis. Five replicates were prepared per volume of plasma as well as five replicates of procedural blanks.

Method

Individual volumes of aliquots were adjusted with aqueous 9 g/L NaCl to 100 μL each and 104 of 5 M aqueous ammonium acetate solution were added. Protein precipitation was done using 200 μL ice cold acetonitrile, samples were shaken for 5 min. at 12° C. and then filtered using an Ultrafree® MC 5.0 μm Filter Unit (Millipore). The precipitate in the filter was subsequently washed with 210 μL water and 400 μL of a mixture of ethanol and dichloromethane (1:2, v:v). After collecting all filtrates of one sample in one collection tube and final centrifugation, 200 μL of the lower phase were evaporated to dryness and redissolved in 200 μL of a mixture of mobile phase A and tetrahydrofurane (1/1, v/v) for further analysis.

All samples were subjected to reversed phase chromatography using a HPLC system (Agilent 1100).

Chromatographic separation was performed at a flow rate of 200 uL/min using a HPLC column (Grom-SIL 80 ODS-7 PH, 4 μm; 60 mm ID: 2 mm) maintained at 35° C. A gradient of mobile phase A and B was used for the separation of the metabolites. Mobile phase A MeOH/H2O/MTBE/0.5% Formic acid 77/18.5/4 (w/w) and B of MTBE/MeOH/H2O/0.5% Formic acid 91/7/1.5 (w/w). The chromatographic gradient was as followed:

Gradient Table:

| @Step | Total Time(min) | Flow Rate(μl/min) | A (%) | B (%) |
|---|---|---|---|---|
| 0 | 0.00 | 200.00 | 100.0 | 0.0 |
| 1 | 0.10 | 200.00 | 100.0 | 0.0 |
| 2 | 0.50 | 200.00 | 60.0 | 40.0 |
| 3 | 5.50 | 200.00 | 0.0 | 100.0 |
| 4 | 6.00 | 200.00 | 0.0 | 100.0 |
| 5 | 6.10 | 200.00 | 100.0 | 0.0 |
| 6 | 7.00 | 200.00 | 100.0 | 0.0 |

Mass spectrometric analysis was performed with a Q-ToF MS (TripleTOF 5600+, AB Sciex, LLC) operating in the positive ion mode using a DuoSpray ion source. The instrument was operated at a mass resolution of 50 000 for ToF MS scans and for product ion scans in the high sensitivity mode. The instrument was automatically calibrated every 10 samples using APCI positive calibration solution delivered via a calibration delivery system (AB Sciex, LLC).

The MS parameters were set as follows: curtain gas, 30 (arbitrary units); ion source gas 25 (arbitrary units); ion source gas 50 (arbitrary units); temperature, 400° C.; ion spray voltage floating, 5500 kV; declustering potential, 100 V.

The SWATH® methods were composed of a TOF MS scan (accumulation time, 100 ms) and a series of product ion scans (accumulation time 20 ms each) of 22 SWATH® precursor selection windows of 1 Da (for examples from m/z 100-122) in the high-sensitivity mode MS/MS experiments were carried out with a rolling collision energy.

The next SWATH® methods consisted of the following 22 Q1 window of 1 Da (from m/z 123 to 150) until m/z 1000.

Data Processing

Data processing activities 1-7 were performed with Expressionist® 10.2 Refiner MS module (commercial software from Genedata AG, Basel, Switzerland) with optimization of activity parameters due to the manual.
1. Chemical Noise subtraction
2. Chromatogram RT Alignment using pairwise alignment
3. Chromatogram Peak Detection
4. Filter on peak validity for peaks present in the majority of the experiments
5. Isotope clustering with a RT tolerance of 0.01 min. and a m/z tolerance of 0.005 Da
6. Adduct Detection focusing on protonation, sodium, potassium, ammonium and water adducts
7. Annotation based on library with a RT tolerance of 0.1 min.
8. Performing of data analysis from R scripts

Step 1. Data Pre-Processing
Description: Gather all individual mass files into a single file to be statistically analyzed (for fragment ion mass data, full scan data is treated separately).

Step 2. Assessing the Linearity of the Measured Peak Areas in Relation to the Dilution of the Samples
Description: Ideally, the amount of a given substance in a sample should be positively correlated with the concentration of that same sample in the measured dilution (higher concentrations should lead to higher measurements of a given substance/analyte), and therefore positively correlated with the concentration of the sample. Given that 5 dilution levels for each mother sample are measured, it is possible to statistically estimate this correlation.
We decided to fit simple linear regression models (SLRMs) [Ref1, Ref 2.] to the peak areas, with the dilution percentages as independent variables.
The advantage of fitting SLRM to the data instead of just calculating a correlation coefficient is that we can also extract the slope of the fitted line. Measurements with negative slopes, slopes close to zero, or correlation coefficients that are too low, might point to analytical problems. Such cases might be filtered out from the dataset if desired.
Statistical Methods:
Simple linear regression model (SLRM) [Ref.1, Ref 2.] for the peak values as response variable and the known dilution (concentration) of the samples as unique explanatory variable, together with an intercept term.

peak value˜a+b*dilutions Formula:
The r², called coefficient of determination that is calculated when fitting these models, is also the square of the sample Pearson correlation coefficient r [Ref. 3] which measures the linear correlation between two variables X and Y. The Pearson correlation coefficient has values between +1 and −1, where 1 is total positive linear correlation, 0 is no linear correlation, and −1 is total negative linear correlation.
The models were fitted using the lm function from the stats package in the R Language and Environment for Statistical Computing [Ref. 4].
R-function: lm( ) [Ref. 5]
Output:
Full scan and fragment ion mass data annotated with the following values:
Pearson correlation coefficient for peak vs dilution
Slope of the fitted regression line for peak vs dilution Histograms describing the distribution of the correlation coefficients.

Step 3. Clustering Retention Time (RT) Values
Description: Clustering the retention time (RT) values from the measured samples at selected dilution levels.
Statistical method:
Optimal k-means clustering in one dimension by dynamic programming, as implemented in the Ckmeans.1d.cp package [Ref.6] and function in R.
This method minimizes the unweighted within-cluster sum of squared distance (L2).
In contrast to the heuristic k-means algorithms, this function optimally assigns elements to k clusters by dynamic programming [Ref. 6]. It minimizes the total of within-cluster sums of squared distances between each element and its corresponding cluster mean.
When a range is provided for the number of clusters, the exact number is determined by Bayesian information criterion. In this case, the range of potential cluster number was set to 1 to 50.

R-Package: Ckmeans.1d.dp

R-Function: Ckmeans.1d.dp

Output: Annotated fragment ion mass file describing which fragment ion belongs to which cluster.

Plots are generated for each SWATH® window (precursor selection window) mass range coloring the individual fragment ions according to their assignment to a given cluster.

a) Calculate cluster width, minimum and maximum cluster border. This step requires no additional statistical method. The limits and range of each cluster are defined, respectively, as the minimum and maximum RT and the range within that cluster.

b) The objective is to define a range of RTs that represents every cluster, which can be used for assigning RT values from the full scan data to the clusters based on fragment ion mass data.

Output: A table containing cluster number, lower and upper bound and range.

Step 4. Relating Full Scan Ions to the RT Clusters Identified on the Fragment Ion Mass Data

Description: The assumption is that peaks with similar masses and similar retention times should come from measuring the same analytes. In this step we relate the measured retention times in the full scan data to the clusters found on fragment ion mass data.

The first step is to link the full scan data to the fragment ion mass data through the mass windows. Full scan measurements within a mass window M will be linked to fragment ion mass clusters identified in that same mass window. Once they align on a given mass window, we focus on the retention times and assign peaks to a cluster, if they have RT values that fall within the range of that given cluster. This peak gets an annotation indicating to which cluster from which mass window it belongs.

This step requires no statistical analysis.

Output: Annotated full scan data file describing which full scan ion belongs to which fragment ion mass data cluster.

Step 5. Annotate the Fragment Ion Mass Data with the Description and Group from the Full Scan Data Description: The idea here is that, by linking the full scan and the fragment ion mass data by mass windows and corresponding clusters, we have identified analytes/peaks that belong together. Therefore, the fragment ion mass data should get the same description and the same group as the data from the full scan. This step helps in the identification of fragments in the fragment ion mass data that belong to the same analyte.

Results

Full Scan Data

TAG (C16:0;C18:1;C18:3) is one of the compound entered in our custom made MS/MS library. TAG (C16:0;C18:1;C18:3) (peak 34309) was successfully identified in the plasma sample as shown in Table 1 with retention time 3.86 min and at m/z 855.7427.

Although the library did not contain the different isotopes of TAG (C16:0;C18:1;C18:3) the method allowed to group several isotopes due to their mass shifts corresponding to the difference between the carbon isotopes 13 C and 12 C (all belonging to cluster 1661). In addition, several adducts were detected and also attributed to TAG (C16:0;C18:1;C18:3). Thus, Peaks 34828 and 34864 correspond to the ammonium adduct of TAG (C16:0;C18:1;C18:3) and isotopic peak of the ammonium adduct of TAG (C16:0;C18:1;C18:3), respectively (cluster 1696). Furthermore a sodium adduct of TAG (C16:0;C18:1;C18:3) and its corresponding isotopic peak were also identified (peaks 34953 and 34987, cluster 1706), and a potassium adduct (peak 35353, cluster 1731). All peaks and clusters belonging to the same metabolite (isotopes and adducts) were grouped by the method correctly to group 147. One Isotope peak m/z 857.7472 (peak 34373) was misidentified by the library search with metabolite TAG (C16:0;C18:1;C18:2) having a similar m/z 857.7593 for its main isotope but a slightly higher RT than the second isotope of group 147 metabolite. However, the grouping method showed clearly that it belongs to group 147 and therefore the library was revealed to be wrong.

A correlation of plasma volume and signal intensity was observed for all different ion forms of TAG (C16:0;C18:1;C18:3) except the sodium adducts and its isotope peak. Correlation of signal intensity to volume of material extracted is a good indicator to distinguish between real metabolite signals and artefacts. Good correlation (correlation coefficient >0.7; RSQ is square of correlation coefficient) was observed for all full scan ions except sodium adduct (see Table 1)

MS/MS SWATH® Data

Since SWATH® acquisition provide the MS/MS ions (fragments) from all precursors detected, it is expected that multiple fragments ions will result from the ionization of TAG (C16:0;C18:1;C18:3).

As the [M+H]⁺ was the analyte used for the library entry of the metabolite TAG (C16:0;C18:1;C18:3) several fragments are expected from this precursor (m/z 855.7427).

Library match was indeed confirmed for 8 fragments. All fragments also demonstrated a good correlation with the volume of plasma extracted (Table 2).

As expected the known fragments that were included in the library were successfully annotated and grouped to the corresponding full scan ion [M+H]⁺. Besides the fragments from the library, 279 fragments resulting from the [M+H]⁺ were identified. Since the library entry is limited to the most abundant fragments, many less intensive fragments are not typically used for identification but can be attributed to a full scan ion (Table 2).

The method also allowed to successfully group fragments from the different precursors for metabolite TAG (C16:0;C18:1;C18:3) in Table 1. In total 882 fragments (Table 1 to 9) were classified in group 147 and a large majority (more than 700) correlated with the volume of plasma, which indicate that only a few peaks that can be attributed as contaminants or low quality were among those fragments classified as TAG (C16:0;C18:1;C18:3) fragments.

TABLE 1
Group of different full scan ions from metabolite TAG (C16:0; C18:1; C18:3) found in the full scan ToF data set

| UnitMassFile_R | Name | Cluster | Group | m.z | RT | m.z. Width | RT. Height | m.z. Min | m.z. Max |
|---|---|---|---|---|---|---|---|---|---|
| 855_14 | Peak_34309 | 1661 | 147 | 855.7427 | 3.851993 | 0.066065 | 0.183317 | 855.7172 | 855.7833 |
| 856_12 | Peak_34342 | 1661 | 147 | 856.7459 | 3.853262 | 0.074367 | 0.183317 | 856.7209 | 856.7952 |
| 857_5 | Peak_34373 | 1661 | 147 | 857.7472 | 3.857774 | 0.066143 | 0.219983 | 857.7209 | 857.7871 |
| 872_12 | Peak_34828 | 1696 | 147 | 872.7689 | 3.857361 | 0.08757 | 0.238317 | 872.7424 | 872.83 |
| 873_12 | Peak_34864 | 1696 | 147 | 873.7717 | 3.858087 | 0.066758 | 0.183317 | 873.7476 | 873.8144 |
| 877_16 | Peak_34953 | 1706 | 147 | 877.7197 | 3.857664 | 0.066908 | 0.128317 | 877.6949 | 877.7618 |
| 878_9 | Peak_34987 | 1706 | 147 | 878.7231 | 3.85731 | 0.075315 | 0.128317 | 878.6946 | 878.7699 |
| 893_11 | Peak_35353 | 1731 | 147 | 893.6948 | 3.861755 | 0.033757 | 0.14665 | 893.6741 | 893.7079 |

| UnitMassFile_R | Name | RT. Min | RT. Max | Library. m.z | Library. RT | Description_FSD | Slope | RSQ |
|---|---|---|---|---|---|---|---|---|
| 855_14 | Peak_34309 | 3.760183 | 3.9435 | 855.7427 | 3.86 | TAG (C16:0, C18:1, C18:3) | 1066.119 | 0.928187 |
| | | | | | | (+H) Survey | | |
| 856_12 | Peak_34342 | 3.760183 | 3.9435 | | | NO DESCRIPTION | 731.2411 | 0.774692 |
| | | | | | | AVAILABLE | | |
| 857_5 | Peak_34373 | 3.778517 | 3.9985 | 857.759267411791 | 3.95 | TAG (C16:0, C18:1, C18:2) | 242.0245 | 0.844661 |
| | | | | | | (+H) Survey | | |
| 872_12 | Peak_34828 | 3.723517 | 3.961833 | | | NO DESCRIPTION | 278.0476 | 0.917623 |
| | | | | | | AVAILABLE | | |
| 873_12 | Peak_34864 | 3.778517 | 3.961833 | | | NO DESCRIPTION | 176.386 | 0.909135 |
| | | | | | | AVAILABLE | | |
| 877_16 | Peak_34953 | 3.79685 | 3.925167 | | | NO DESCRIPTION | 14.08083 | 0.194266 |
| | | | | | | AVAILABLE | | |
| 878_9 | Peak_34987 | 3.79685 | 3.925167 | | | NO DESCRIPTION | 8.37732 | 0.184197 |
| | | | | | | AVAILABLE | | |
| 893_11 | Peak_35353 | 3.79685 | 3.9435 | | | NO DESCRIPTION | 4.470517 | 0.635766 |
| | | | | | | AVAILABLE | | |

TABLE 2
Group of different fragment ions from precursor m/z 855.7427 corresponding
to ionized TAG (C16:0; C18:1; C18:3) found in the SWATH ® window m/z 855

| UnitMassFile_R | Name | Cluster | Group_FSD | m.z | RT | m.z. Min | m.z. Max | RT. Min | RT. Max |
|---|---|---|---|---|---|---|---|---|---|
| 855_14 | Peak_358 | 333 | 147 | 599.5031 | 3.875739 | 599.4572 | 599.547 | 3.805008 | 3.949606 |
| 855_14 | Peak_341 | 318 | 147 | 543.477 | 3.875349 | 543.4375 | 543.5264 | 3.805008 | 3.949606 |
| 855_14 | Peak_333 | 310 | 147 | 525.4668 | 3.875556 | 525.4288 | 525.5162 | 3.805008 | 3.949606 |
| 855_14 | Peak_384 | 351 | 147 | 855.7444 | 3.875481 | 855.6864 | 855.8061 | 3.805008 | 3.935768 |
| 855_14 | Peak_197 | 189 | 147 | 313.274 | 3.876052 | 313.2419 | 313.3043 | 3.805008 | 3.949606 |
| 855_14 | Peak_003 | 13 | 147 | 95.0869 | 3.875207 | 95.07782 | 95.09984 | 3.805008 | 3.949606 |
| 855_14 | Peak_132 | 130 | 147 | 261.2216 | 3.875769 | 261.2048 | 261.2368 | 3.805008 | 3.949606 |
| 855_14 | Peak_014 | 21 | 147 | 109.1021 | 3.875244 | 109.0931 | 109.1153 | 3.805008 | 3.949606 |
| 855_14 | Peak_359 | 334 | 147 | 600.5056 | 3.876192 | 600.4598 | 600.5567 | 3.81848 | 3.935768 |
| 855_14 | Peak_020 | 27 | 147 | 121.1017 | 3.875524 | 121.0915 | 121.1132 | 3.805008 | 3.935768 |
| 855_14 | Peak_353 | 328 | 147 | 577.5188 | 3.876714 | 577.4785 | 577.5633 | 3.81848 | 3.935768 |
| 855_14 | Peak_022 | 29 | 147 | 123.1173 | 3.875267 | 123.1066 | 123.1301 | 3.81848 | 3.949606 |
| 855_14 | Peak_030 | 37 | 147 | 135.1172 | 3.875103 | 135.1066 | 135.1296 | 3.805008 | 3.935768 |
| 855_14 | Peak_116 | 114 | 147 | 243.211 | 3.875922 | 243.1962 | 243.2314 | 3.81848 | 3.935768 |
| 855_14 | Peak_042 | 49 | 147 | 149.1327 | 3.875685 | 149.1211 | 149.1452 | 3.81848 | 3.935768 |
| 855_14 | Peak_005 | 15 | 147 | 97.10202 | 3.875431 | 97.09375 | 97.11183 | 3.81848 | 3.935768 |
| 855_14 | Peak_390 | 353 | 147 | 856.7443 | 3.878256 | 856.69 | 856.7851 | 3.831584 | 3.935768 |
| 855_14 | Peak_054 | 61 | 147 | 175.1485 | 3.875655 | 175.1358 | 175.1638 | 3.81848 | 3.935768 |
| 855_14 | Peak_002 | 12 | 147 | 93.07099 | 3.875628 | 93.06262 | 93.08168 | 3.81848 | 3.935768 |
| 855_14 | Peak_201 | 193 | 147 | 317.2472 | 3.876426 | 317.2241 | 317.2618 | 3.81848 | 3.949606 |
| 855_14 | Peak_362 | 337 | 147 | 617.5134 | 3.875907 | 617.4682 | 617.5664 | 3.81848 | 3.949606 |
| 855_14 | Peak_063 | 68 | 147 | 187.1484 | 3.876009 | 187.1347 | 187.1637 | 3.81848 | 3.949606 |
| 855_14 | Peak_012 | 19 | 147 | 107.0863 | 3.875432 | 107.0768 | 107.0973 | 3.81848 | 3.935768 |
| 855_14 | Peak_112 | 111 | 147 | 239.237 | 3.876533 | 239.2217 | 239.2544 | 3.81848 | 3.949606 |
| 855_14 | Peak_053 | 60 | 147 | 173.133 | 3.875763 | 173.1204 | 173.1483 | 3.81848 | 3.935768 |
| 855_14 | Peak_028 | 35 | 147 | 133.1016 | 3.875947 | 133.0912 | 133.114 | 3.81848 | 3.935768 |
| 855_14 | Peak_019 | 26 | 147 | 119.0862 | 3.874826 | 119.0776 | 119.0976 | 3.81848 | 3.935768 |
| 855_14 | Peak_351 | 326 | 147 | 575.5029 | 3.875167 | 575.462 | 575.5467 | 3.81848 | 3.935768 |
| 855_14 | Peak_179 | 172 | 147 | 289.2527 | 3.875726 | 289.2377 | 289.2689 | 3.81848 | 3.935768 |
| 855_14 | Peak_159 | 152 | 147 | 275.2368 | 3.875606 | 275.2241 | 275.2499 | 3.81848 | 3.935768 |
| 855_14 | Peak_289 | 269 | 147 | 417.3728 | 3.876323 | 417.3544 | 417.4034 | 3.81848 | 3.935768 |
| 855_14 | Peak_032 | 39 | 147 | 137.1327 | 3.875715 | 137.1223 | 137.1455 | 3.81848 | 3.935768 |
| 855_14 | Peak_349 | 325 | 147 | 573.487 | 3.874642 | 573.4457 | 573.5268 | 3.81848 | 3.935768 |
| 855_14 | Peak_327 | 305 | 147 | 507.4545 | 3.876062 | 507.4055 | 507.4914 | 3.81848 | 3.935768 |
| 855_14 | Peak_065 | 70 | 147 | 189.164 | 3.876001 | 189.1503 | 189.1794 | 3.81848 | 3.935768 |
| 855_14 | Peak_072 | 76 | 147 | 201.1641 | 3.875988 | 201.1496 | 201.1796 | 3.81848 | 3.935768 |
| 855_14 | Peak_047 | 54 | 147 | 161.1325 | 3.8759 | 161.1209 | 161.146 | 3.81848 | 3.935768 |
| 855_14 | Peak_049 | 56 | 147 | 163.1483 | 3.875829 | 163.1375 | 163.1627 | 3.81848 | 3.935768 |
| 855_14 | Peak_271 | 252 | 147 | 387.3628 | 3.87588 | 387.3473 | 387.3889 | 3.81848 | 3.935768 |
| 855_14 | Peak_035 | 42 | 147 | 145.1015 | 3.875017 | 145.0911 | 145.1132 | 3.81848 | 3.935768 |
| 855_14 | Peak_263 | 244 | 147 | 373.3474 | 3.876203 | 373.3333 | 373.3769 | 3.81848 | 3.935768 |
| 855_14 | Peak_190 | 183 | 147 | 303.2687 | 3.876093 | 303.2549 | 303.2869 | 3.831584 | 3.935768 |
| 855_14 | Peak_038 | 45 | 147 | 147.1171 | 3.875729 | 147.106 | 147.13 | 3.81848 | 3.935768 |
| 855_14 | Peak_354 | 329 | 147 | 581.4911 | 3.876169 | 581.4472 | 581.5289 | 3.81848 | 3.935768 |
| 855_14 | Peak_361 | 330 | 147 | 615.4988 | 3.875649 | 615.453 | 615.5371 | 3.81848 | 3.935768 |
| 855_14 | Peak_373 | 347 | 147 | 837.732 | 3.874998 | 837.6723 | 837.7907 | 3.81848 | 3.935768 |
| 855_14 | Peak_084 | 86 | 147 | 215.1796 | 3.876051 | 215.1649 | 215.1959 | 3.81848 | 3.935768 |
| 855_14 | Peak_276 | 257 | 147 | 399.3622 | 3.875947 | 399.3455 | 399.3907 | 3.81848 | 3.935768 |
| 855_14 | Peak_073 | 77 | 147 | 203.1795 | 3.87767 | 203.1665 | 203.1947 | 3.81848 | 3.935768 |
| 855_14 | Peak_027 | 34 | 147 | 131.086 | 3.875928 | 131.0764 | 131.0974 | 3.81848 | 3.935768 |
| 855_14 | Peak_056 | 63 | 147 | 177.1639 | 3.875367 | 177.1515 | 177.1778 | 3.81848 | 3.935768 |
| 855_14 | Peak_046 | 53 | 147 | 159.117 | 3.875107 | 159.1062 | 159.1293 | 3.81848 | 3.935768 |
| 855_14 | Peak_328 | 306 | 147 | 515.4088 | 3.876628 | 515.3728 | 515.4337 | 3.81848 | 3.935768 |
| 855_14 | Peak_011 | 18 | 147 | 105.0706 | 3.875296 | 105.062 | 105.0808 | 3.81848 | 3.935768 |
| 855_14 | Peak_253 | 236 | 147 | 359.3315 | 3.875971 | 359.3179 | 359.3553 | 3.81848 | 3.935768 |
| 855_14 | Peak_222 | 213 | 147 | 339.291 | 3.875828 | 339.2784 | 339.3174 | 3.81848 | 3.935768 |
| 855_14 | Peak_121 | 119 | 147 | 247.2422 | 3.875657 | 247.2301 | 247.2589 | 3.81848 | 3.935768 |
| 855_14 | Peak_282 | 262 | 147 | 403.3573 | 3.876385 | 403.3412 | 403.3866 | 3.81848 | 3.935768 |
| 855_14 | Peak_324 | 302 | 147 | 501.3916 | 3.875677 | 501.3665 | 501.4108 | 3.81848 | 3.935768 |
| 855_14 | Peak_285 | 265 | 147 | 413.3786 | 3.875698 | 413.3643 | 413.4074 | 3.831584 | 3.935768 |
| 855_14 | Peak_279 | 260 | 147 | 401.3778 | 3.876363 | 401.3621 | 401.4045 | 3.81848 | 3.935768 |
| 855_14 | Peak_136 | 134 | 147 | 263.237 | 3.875171 | 263.2254 | 263.2506 | 3.81848 | 3.935768 |
| 855_14 | Peak_103 | 104 | 147 | 233.2264 | 3.875888 | 233.2146 | 233.2426 | 3.81848 | 3.935768 |
| 855_14 | Peak_100 | 101 | 147 | 229.1952 | 3.875951 | 229.1797 | 229.2117 | 3.831584 | 3.935768 |
| 855_14 | Peak_044 | 51 | 147 | 151.1481 | 3.876458 | 151.1377 | 151.1602 | 3.81848 | 3.935768 |
| 855_14 | Peak_118 | 116 | 147 | 245.2265 | 3.87629 | 245.2145 | 245.2432 | 3.831584 | 3.935768 |
| 855_14 | Peak_311 | 289 | 147 | 459.3826 | 3.876669 | 459.3642 | 459.4096 | 3.81848 | 3.935768 |
| 855_14 | Peak_233 | 221 | 147 | 345.2782 | 3.875287 | 345.2584 | 345.2925 | 3.81848 | 3.935768 |
| 855_14 | Peak_067 | 72 | 147 | 191.1794 | 3.875642 | 191.1687 | 191.1922 | 3.831584 | 3.935768 |
| 855_14 | Peak_062 | 67 | 147 | 185.1326 | 3.875711 | 185.1166 | 185.1473 | 3.81848 | 3.935768 |
| 855_14 | Peak_303 | 281 | 147 | 443.3885 | 3.876438 | 443.3659 | 443.4194 | 3.831584 | 3.935768 |
| 855_14 | Peak_016 | 23 | 147 | 111.1172 | 3.875605 | 111.1089 | 111.1268 | 3.81848 | 3.935768 |
| 855_14 | Peak_294 | 274 | 147 | 429.3718 | 3.876089 | 429.3459 | 429.3957 | 3.81848 | 3.935768 |
| 855_14 | Peak_202 | 194 | 147 | 317.2843 | 3.876045 | 317.2744 | 317.3046 | 3.81848 | 3.935768 |
| 855_14 | Peak_298 | 277 | 147 | 431.3886 | 3.876588 | 431.3752 | 431.4162 | 3.831584 | 3.935768 |
| 855_14 | Peak_204 | 195 | 147 | 319.2633 | 3.875641 | 319.2486 | 319.2814 | 3.831584 | 3.935768 |
| 855_14 | Peak_070 | 75 | 147 | 199.1477 | 3.876214 | 199.1267 | 199.1626 | 3.831584 | 3.935768 |
| 855_14 | Peak_346 | 322 | 147 | 559.4707 | 3.876013 | 559.4437 | 559.5071 | 3.81848 | 3.935768 |
| 855_14 | Peak_214 | 205 | 147 | 331.2619 | 3.875869 | 331.2432 | 331.274 | 3.81848 | 3.935768 |
| 855_14 | Peak_305 | 283 | 147 | 445.3657 | 3.876721 | 445.3506 | 445.3804 | 3.831584 | 3.935768 |
| 855_14 | Peak_191 | 184 | 147 | 305.247 | 3.875867 | 305.232 | 305.2616 | 3.81848 | 3.949606 |
| 855_14 | Peak_086 | 88 | 147 | 217.195 | 3.875772 | 217.1821 | 217.2092 | 3.81848 | 3.935768 |
| 855_14 | Peak_315 | 293 | 147 | 473.3988 | 3.875756 | 473.383 | 473.429 | 3.831584 | 3.935768 |
| 855_14 | Peak_292 | 272 | 147 | 427.3548 | 3.875863 | 427.336 | 427.3681 | 3.831584 | 3.935768 |
| 855_14 | Peak_127 | 125 | 147 | 257.2258 | 3.87566 | 257.2142 | 257.2369 | 3.831584 | 3.935768 |
| 855_14 | Peak_217 | 208 | 147 | 333.2792 | 3.876008 | 333.2655 | 333.299 | 3.81848 | 3.935768 |
| 855_14 | Peak_075 | 79 | 147 | 205.1948 | 3.875068 | 205.1835 | 205.2077 | 3.831584 | 3.935768 |
| 855_14 | Peak_301 | 279 | 147 | 441.3717 | 3.876084 | 441.3442 | 441.3917 | 3.831584 | 3.935768 |
| 855_14 | Peak_091 | 93 | 147 | 221.2265 | 3.876044 | 221.2155 | 221.2406 | 3.81848 | 3.935768 |
| 855_14 | Peak_083 | 85 | 147 | 213.1625 | 3.876313 | 213.1406 | 213.1797 | 3.81848 | 3.935768 |
| 855_14 | Peak_342 | 319 | 147 | 544.4781 | 3.876639 | 544.4449 | 544.5108 | 3.81848 | 3.935768 |
| 855_14 | Peak_181 | 174 | 147 | 291.2315 | 3.876918 | 291.2167 | 291.2456 | 3.831584 | 3.935768 |
| 855_14 | Peak_200 | 192 | 147 | 315.2677 | 3.876331 | 315.2537 | 315.2812 | 3.81848 | 3.935768 |
| 855_14 | Peak_319 | 297 | 147 | 487.375 | 3.875787 | 487.3506 | 487.3911 | 3.831584 | 3.935768 |
| 855_14 | Peak_052 | 59 | 147 | 171.1173 | 3.876558 | 171.1056 | 171.1297 | 3.81848 | 3.935768 |
| 855_14 | Peak_101 | 102 | 147 | 231.2108 | 3.875304 | 231.1992 | 231.225 | 3.831584 | 3.935768 |
| 855_14 | Peak_088 | 90 | 147 | 219.2108 | 3.874593 | 219.1983 | 219.2255 | 3.831584 | 3.935768 |
| 855_14 | Peak_286 | 266 | 147 | 415.3547 | 3.875659 | 415.3325 | 415.3699 | 3.831584 | 3.935768 |
| 855_14 | Peak_252 | 235 | 147 | 359.2934 | 3.875178 | 359.2751 | 359.3045 | 3.831584 | 3.935768 |
| 855_14 | Peak_348 | 324 | 147 | 563.4912 | 3.876079 | 563.4472 | 563.5343 | 3.831584 | 3.935768 |
| 855_14 | Peak_234 | 222 | 147 | 345.3157 | 3.876628 | 345.3057 | 345.3345 | 3.831584 | 3.935768 |
| 855_14 | Peak_187 | 180 | 147 | 301.2525 | 3.875555 | 301.2402 | 301.2647 | 3.831584 | 3.935768 |
| 855_14 | Peak_269 | 250 | 147 | 385.347 | 3.875716 | 385.333 | 385.3663 | 3.831584 | 3.935768 |
| 855_14 | Peak_272 | 253 | 147 | 389.3415 | 3.87602 | 389.3278 | 389.3612 | 3.831584 | 3.935768 |
| 855_14 | Peak_161 | 154 | 147 | 277.2161 | 3.876207 | 277.2017 | 277.2299 | 3.831584 | 3.935768 |
| 855_14 | Peak_360 | 335 | 147 | 601.5185 | 3.871801 | 601.5014 | 601.5429 | 3.81848 | 3.935768 |
| 855_14 | Peak_176 | 169 | 147 | 287.2368 | 3.883533 | 287.2247 | 287.2486 | 3.831584 | 3.949606 |
| 855_14 | Peak_335 | 312 | 147 | 529.4242 | 3.87616 | 529.3901 | 529.4551 | 3.831584 | 3.935768 |
| 855_14 | Peak_221 | 212 | 147 | 339.2689 | 3.874735 | 339.2472 | 339.2784 | 3.831584 | 3.935768 |
| 855_14 | Peak_213 | 204 | 147 | 329.2846 | 3.876782 | 329.2731 | 329.3013 | 3.831584 | 3.935768 |
| 855_14 | Peak_215 | 206 | 147 | 331.3002 | 3.87584 | 331.2894 | 331.3203 | 3.831584 | 3.935768 |
| 855_14 | Peak_099 | 100 | 147 | 227.1781 | 3.876163 | 227.1542 | 227.1946 | 3.81848 | 3.935768 |
| 855_14 | Peak_130 | 128 | 147 | 259.2425 | 3.876417 | 259.2307 | 259.258 | 3.81848 | 3.935768 |
| 855_14 | Peak_157 | 150 | 147 | 273.2581 | 3.875793 | 273.2466 | 273.2746 | 3.831584 | 3.935768 |
| 855_14 | Peak_050 | 57 | 147 | 165.1636 | 3.876637 | 165.1539 | 165.1738 | 3.831584 | 3.935768 |
| 855_14 | Peak_001 | 11 | 147 | 91.05539 | 3.875221 | 91.04745 | 91.06496 | 3.81848 | 3.935768 |
| 855_14 | Peak_114 | 112 | 147 | 241.1943 | 3.876231 | 241.1774 | 241.2103 | 3.81848 | 3.935768 |
| 855_14 | Peak_185 | 178 | 147 | 299.2365 | 3.876915 | 299.22 | 299.2493 | 3.81848 | 3.935768 |
| 855_14 | Peak_154 | 148 | 147 | 271.242 | 3.876168 | 271.2297 | 271.2576 | 3.831584 | 3.935768 |
| 855_14 | Peak_297 | 276 | 147 | 431.3507 | 3.876613 | 431.3371 | 431.3635 | 3.831584 | 3.935768 |
| 855_14 | Peak_367 | 342 | 147 | 729.6386 | 3.877036 | 729.6184 | 729.6755 | 3.831584 | 3.935768 |
| 855_14 | Peak_308 | 286 | 147 | 455.3878 | 3.87607 | 455.3644 | 455.4156 | 3.831584 | 3.935768 |
| 855_14 | Peak_236 | 223 | 147 | 347.2943 | 3.876338 | 347.2809 | 347.3125 | 3.831584 | 3.935768 |
| 855_14 | Peak_293 | 273 | 147 | 427.3941 | 3.876576 | 427.3827 | 427.4177 | 3.831584 | 3.935768 |
| 855_14 | Peak_208 | 199 | 147 | 327.2681 | 3.877283 | 327.2528 | 327.2834 | 3.831584 | 3.935768 |
| 855_14 | Peak_150 | 145 | 147 | 269.226 | 3.881928 | 269.2133 | 269.2411 | 3.831584 | 3.949606 |
| 855_14 | Peak_369 | 344 | 147 | 743.6539 | 3.87572 | 743.6359 | 743.6898 | 3.831584 | 3.935768 |
| 855_14 | Peak_339 | 316 | 147 | 541.4601 | 3.875367 | 541.4289 | 541.4979 | 3.81848 | 3.935768 |
| 855_14 | Peak_334 | 311 | 147 | 526.4673 | 3.875456 | 526.4356 | 526.4971 | 3.831584 | 3.935768 |
| 855_14 | Peak_307 | 285 | 147 | 451.3757 | 3.876306 | 451.3401 | 451.4031 | 3.831584 | 3.935768 |
| 855_14 | Peak_120 | 118 | 147 | 247.206 | 3.875307 | 247.1946 | 247.2167 | 3.831584 | 3.935768 |
| 855_14 | Peak_265 | 246 | 147 | 375.3251 | 3.87609 | 375.3132 | 375.3433 | 3.831584 | 3.935768 |
| 855_14 | Peak_140 | 138 | 147 | 265.2525 | 3.877959 | 265.2399 | 265.2675 | 3.831584 | 3.935768 |
| 855_14 | Peak_229 | 218 | 147 | 343.2617 | 3.875724 | 343.2445 | 343.2732 | 3.831584 | 3.935768 |
| 855_14 | Peak_309 | 287 | 147 | 457.4031 | 3.876887 | 457.3848 | 457.43 | 3.831584 | 3.935768 |
| 855_14 | Peak_133 | 131 | 147 | 261.2574 | 3.875964 | 261.2505 | 261.271 | 3.831584 | 3.935768 |
| 855_14 | Peak_129 | 127 | 147 | 259.2061 | 3.876457 | 259.1921 | 259.2171 | 3.831584 | 3.935768 |
| 855_14 | Peak_320 | 298 | 147 | 487.4147 | 3.876105 | 487.4036 | 487.441 | 3.831584 | 3.935768 |
| 855_14 | Peak_106 | 107 | 147 | 235.2418 | 3.875349 | 235.2301 | 235.254 | 3.831584 | 3.935768 |
| 855_14 | Peak_331 | 308 | 147 | 523.4516 | 3.87484 | 523.4247 | 523.4828 | 3.81848 | 3.935768 |
| 855_14 | Peak_156 | 149 | 147 | 273.2209 | 3.876359 | 273.2069 | 273.2326 | 3.831584 | 3.935768 |
| 855_14 | Peak_125 | 123 | 147 | 255.2106 | 3.875772 | 255.1966 | 255.2259 | 3.831584 | 3.935768 |
| 855_14 | Peak_278 | 259 | 147 | 401.3393 | 3.87514 | 401.3197 | 401.3508 | 3.831584 | 3.935768 |
| 855_14 | Peak_300 | 278 | 147 | 437.369 | 3.876688 | 437.35 | 437.3943 | 3.831584 | 3.935768 |
| 855_14 | Peak_173 | 166 | 147 | 285.2217 | 3.876107 | 285.2068 | 285.233 | 3.831584 | 3.935768 |
| 855_14 | Peak_261 | 242 | 147 | 371.3311 | 3.875809 | 371.3205 | 371.3477 | 3.831584 | 3.935768 |
| 855_14 | Peak_248 | 232 | 147 | 357.2779 | 3.876952 | 357.2606 | 357.2899 | 3.831584 | 3.935768 |
| 855_14 | Peak_174 | 167 | 147 | 285.258 | 3.876389 | 285.2473 | 285.2736 | 3.831584 | 3.935768 |
| 855_14 | Peak_212 | 203 | 147 | 329.2476 | 3.876935 | 329.2296 | 329.2603 | 3.831584 | 3.935768 |
| 855_14 | Peak_260 | 241 | 147 | 371.293 | 3.876753 | 371.277 | 371.3042 | 3.831584 | 3.935768 |
| 855_14 | Peak_225 | 215 | 147 | 341.2826 | 3.876453 | 341.2651 | 341.299 | 3.831584 | 3.935768 |
| 855_14 | Peak_343 | 320 | 147 | 549.4859 | 3.876209 | 549.4527 | 549.5156 | 3.831584 | 3.935768 |
| 855_14 | Peak_284 | 264 | 147 | 413.3393 | 3.875794 | 413.3242 | 413.35 | 3.831584 | 3.935768 |
| 855_14 | Peak_182 | 175 | 147 | 293.2474 | 3.875458 | 293.234 | 293.263 | 3.831584 | 3.935768 |
| 855_14 | Peak_262 | 243 | 147 | 373.3076 | 3.875295 | 373.2924 | 373.3169 | 3.831584 | 3.935768 |
| 855_14 | Peak_256 | 238 | 147 | 361.3102 | 3.876871 | 361.2979 | 361.3301 | 3.831584 | 3.935768 |
| 855_14 | Peak_135 | 133 | 147 | 263.2008 | 3.877293 | 263.1887 | 263.2116 | 3.831584 | 3.935768 |
| 855_14 | Peak_128 | 126 | 147 | 257.246 | 3.877093 | 257.2391 | 257.2595 | 3.831584 | 3.935768 |
| 855_14 | Peak_193 | 186 | 147 | 311.2358 | 3.87546 | 311.2216 | 311.2465 | 3.831584 | 3.935768 |
| 855_14 | Peak_057 | 64 | 147 | 179.1795 | 3.876579 | 179.1693 | 179.1901 | 3.831584 | 3.935768 |
| 855_14 | Peak_264 | 245 | 147 | 375.2875 | 3.876598 | 375.2722 | 375.2995 | 3.831584 | 3.935768 |
| 855_14 | Peak_102 | 103 | 147 | 233.1912 | 3.876418 | 233.1801 | 233.1995 | 3.831584 | 3.935768 |
| 855_14 | Peak_275 | 256 | 147 | 399.3239 | 3.876099 | 399.306 | 399.3343 | 3.831584 | 3.935768 |
| 855_14 | Peak_169 | 162 | 147 | 283.2417 | 3.876177 | 283.2293 | 283.2578 | 3.831584 | 3.935768 |
| 855_14 | Peak_314 | 292 | 147 | 473.3586 | 3.876394 | 473.3369 | 473.3707 | 3.831584 | 3.935768 |
| 855_14 | Peak_283 | 263 | 147 | 409.3447 | 3.876598 | 409.3306 | 409.3592 | 3.831584 | 3.935768 |
| 855_14 | Peak_313 | 291 | 147 | 465.3889 | 3.876555 | 465.3525 | 465.4195 | 3.831584 | 3.935768 |
| 855_14 | Peak_249 | 233 | 147 | 357.3147 | 3.875606 | 357.3033 | 357.3299 | 3.831584 | 3.935768 |
| 855_14 | Peak_160 | 153 | 147 | 275.2739 | 3.876466 | 275.2663 | 275.2874 | 3.831584 | 3.935768 |
| 855_14 | Peak_268 | 249 | 147 | 385.3086 | 3.876326 | 385.2915 | 385.3192 | 3.831584 | 3.935768 |
| 855_14 | Peak_206 | 197 | 147 | 325.2511 | 3.87644 | 325.2361 | 325.2615 | 3.831584 | 3.935768 |
| 855_14 | Peak_199 | 191 | 147 | 315.2316 | 3.877785 | 315.2186 | 315.2411 | 3.831584 | 3.935768 |
| 855_14 | Peak_177 | 170 | 147 | 287.2735 | 3.876443 | 287.263 | 287.2893 | 3.831584 | 3.935768 |
| 855_12 | Peak_357 | 332 | 147 | 598.4903 | 3.819939 | 598.4623 | 598.5141 | 3.775677 | 3.92083 |
| 855_14 | Peak_069 | 74 | 147 | 197.1324 | 3.875697 | 197.12 | 197.1438 | 3.831584 | 3.92083 |
| 855_14 | Peak_230 | 219 | 147 | 343.2995 | 3.876697 | 343.2889 | 343.3151 | 3.831584 | 3.935768 |
| 855_14 | Peak_163 | 156 | 147 | 279.2302 | 3.876488 | 279.2053 | 279.2477 | 3.831584 | 3.935768 |
| 855_14 | Peak_220 | 211 | 147 | 337.2743 | 3.875053 | 337.2586 | 337.2975 | 3.831584 | 3.935768 |
| 855_14 | Peak_368 | 343 | 147 | 743.619 | 3.876393 | 743.582 | 743.6359 | 3.831584 | 3.935768 |
| 855_14 | Peak_287 | 267 | 147 | 415.3935 | 3.874978 | 415.3843 | 415.4102 | 3.831584 | 3.92083 |
| 855_14 | Peak_218 | 209 | 147 | 335.2577 | 3.87688 | 335.2397 | 335.2707 | 3.846155 | 3.935768 |
| 855_14 | Peak_192 | 185 | 147 | 307.2627 | 3.875683 | 307.2501 | 307.2798 | 3.831584 | 3.935768 |
| 855_14 | Peak_117 | 115 | 147 | 245.1905 | 3.874953 | 245.1791 | 245.2012 | 3.831584 | 3.935768 |
| 855_14 | Peak_033 | 40 | 147 | 139.1118 | 3.875745 | 139.103 | 139.1213 | 3.831584 | 3.935768 |
| 855_14 | Peak_370 | 345 | 147 | 757.6427 | 3.875806 | 757.5926 | 757.6975 | 3.831584 | 3.935768 |
| 855_14 | Peak_097 | 99 | 147 | 225.164 | 3.876172 | 225.1524 | 225.1757 | 3.831584 | 3.92083 |
| 855_14 | Peak_244 | 228 | 147 | 355.2981 | 3.875903 | 355.281 | 355.3129 | 3.831584 | 3.935768 |
| 855_14 | Peak_270 | 251 | 147 | 387.3233 | 3.876309 | 387.3056 | 387.3334 | 3.831584 | 3.935768 |
| 855_14 | Peak_322 | 300 | 147 | 497.4711 | 3.877293 | 497.4519 | 497.4991 | 3.831584 | 3.935768 |
| 855_14 | Peak_158 | 151 | 147 | 275.2011 | 3.876034 | 275.189 | 275.2101 | 3.831584 | 3.935768 |
| 855_14 | Peak_255 | 237 | 147 | 361.274 | 3.877732 | 361.2577 | 361.2872 | 3.831584 | 3.935768 |
| 855_14 | Peak_024 | 31 | 147 | 125.0963 | 3.876044 | 125.0879 | 125.1052 | 3.831584 | 3.935768 |
| 855_14 | Peak_267 | 248 | 147 | 381.3134 | 3.876614 | 381.2982 | 381.3258 | 3.846155 | 3.935768 |
| 855_14 | Peak_153 | 147 | 147 | 271.2058 | 3.876187 | 271.1925 | 271.2158 | 3.831584 | 3.92083 |
| 855_14 | Peak_216 | 207 | 147 | 333.2419 | 3.876912 | 333.2269 | 333.2526 | 3.831584 | 3.935768 |
| 855_14 | Peak_018 | 25 | 147 | 117.0705 | 3.874187 | 117.0622 | 117.0805 | 3.831584 | 3.935768 |
| 855_14 | Peak_189 | 182 | 147 | 303.2313 | 3.875567 | 303.2156 | 303.2402 | 3.831584 | 3.935768 |
| 855_14 | Peak_087 | 89 | 147 | 219.1756 | 3.877812 | 219.1649 | 219.1858 | 3.846155 | 3.935768 |
| 855_14 | Peak_290 | 270 | 147 | 423.3595 | 3.877164 | 423.3391 | 423.3856 | 3.831584 | 3.935768 |
| 855_14 | Peak_318 | 296 | 147 | 479.4007 | 3.876041 | 479.3628 | 479.4339 | 3.831584 | 3.935768 |
| 855_14 | Peak_306 | 284 | 147 | 445.4039 | 3.876989 | 445.3953 | 445.4251 | 3.831584 | 3.935768 |
| 855_14 | Peak_123 | 121 | 147 | 249.2215 | 3.877373 | 249.2115 | 249.2316 | 3.831584 | 3.935768 |
| 855_14 | Peak_273 | 254 | 147 | 395.3294 | 3.876278 | 395.3108 | 395.3445 | 3.846155 | 3.935768 |
| 855_14 | Peak_082 | 84 | 147 | 211.1485 | 3.875408 | 211.1361 | 211.1607 | 3.831584 | 3.92083 |
| 855_14 | Peak_122 | 120 | 147 | 249.185 | 3.876884 | 249.1736 | 249.1959 | 3.831584 | 3.935768 |
| 855_14 | Peak_074 | 78 | 147 | 205.1592 | 3.877061 | 205.1491 | 205.1693 | 3.831584 | 3.935768 |
| 855_14 | Peak_241 | 225 | 147 | 353.2836 | 3.878386 | 353.2698 | 353.2963 | 3.846155 | 3.935768 |
| 855_14 | Peak_111 | 110 | 147 | 239.1806 | 3.876335 | 239.1671 | 239.1977 | 3.831584 | 3.935768 |
| 855_14 | Peak_124 | 122 | 147 | 249.258 | 3.87616 | 249.2472 | 249.2717 | 3.831584 | 3.935768 |
| 855_14 | Peak_205 | 196 | 147 | 321.2782 | 3.875148 | 321.2669 | 321.2948 | 3.831584 | 3.92083 |
| 855_14 | Peak_025 | 32 | 147 | 125.1323 | 3.876922 | 125.1242 | 125.1416 | 3.831584 | 3.935768 |
| 855_14 | Peak_126 | 124 | 147 | 257.1907 | 3.877293 | 257.178 | 257.2006 | 3.831584 | 3.935768 |
| 855_14 | Peak_323 | 301 | 147 | 499.451 | 3.873621 | 499.4278 | 499.4814 | 3.831584 | 3.935768 |
| 855_14 | Peak_337 | 314 | 147 | 535.4697 | 3.877516 | 535.435 | 535.4938 | 3.831584 | 3.935768 |
| 855_14 | Peak_045 | 52 | 147 | 157.101 | 3.876484 | 157.0935 | 157.1112 | 3.831584 | 3.935768 |
| 855_14 | Peak_104 | 105 | 147 | 235.1697 | 3.876504 | 235.1587 | 235.1804 | 3.831584 | 3.92083 |
| 855_14 | Peak_162 | 155 | 147 | 277.2529 | 3.875813 | 277.244 | 277.2652 | 3.831584 | 3.935768 |
| 855_14 | Peak_371 | 346 | 147 | 771.6455 | 3.876607 | 771.6124 | 771.6673 | 3.846155 | 3.935768 |
| 855_14 | Peak_184 | 177 | 147 | 297.2759 | 3.8775 | 297.2674 | 297.2917 | 3.846155 | 3.935768 |
| 855_14 | Peak_291 | 271 | 147 | 425.3784 | 3.876834 | 425.3658 | 425.3978 | 3.846155 | 3.935768 |
| 855_14 | Peak_139 | 137 | 147 | 265.2156 | 3.876979 | 265.2055 | 265.2261 | 3.831584 | 3.935768 |
| 855_14 | Peak_186 | 179 | 147 | 299.2737 | 3.877186 | 299.2639 | 299.2884 | 3.831584 | 3.92083 |
| 855_14 | Peak_007 | 17 | 147 | 99.11756 | 3.877245 | 99.11114 | 99.1252 | 3.831584 | 3.92083 |
| 855_14 | Peak_356 | 331 | 147 | 597.4862 | 3.867525 | 597.4578 | 597.513 | 3.789976 | 3.92083 |
| 855_I4 | Peak_296 | 275 | 147 | 431.3138 | 3.876567 | 431.2961 | 431.3254 | 3.831584 | 3.92083 |
| 855_14 | Peak_194 | 187 | 147 | 311.2654 | 3.873944 | 311.2539 | 311.2863 | 3.81848 | 3.935768 |
| 855_14 | Peak_078 | 82 | 147 | 207.2105 | 3.876711 | 207.2002 | 207.2226 | 3.831584 | 3.935768 |
| 855_14 | Peak_066 | 71 | 147 | 191.1443 | 3.875442 | 191.1336 | 191.1551 | 3.831584 | 3.92083 |
| 855_14 | Peak_288 | 268 | 147 | 417.2963 | 3.877499 | 417.2794 | 417.3082 | 3.831584 | 3.935768 |
| 855_14 | Peak_006 | 16 | 147 | 99.08098 | 3.875319 | 99.07321 | 99.08866 | 3.831584 | 3.92083 |
| 855_14 | Peak_366 | 341 | 147 | 729.5982 | 3.877454 | 729.5726 | 729.6107 | 3.831584 | 3.935768 |
| 855_14 | Peak_347 | 323 | 147 | 561.4866 | 3.876156 | 561.462 | 561.5156 | 3.831584 | 3.92083 |
| 855_14 | Peak_188 | 181 | 147 | 301.2885 | 3.878431 | 301.2794 | 301.3015 | 3.846155 | 3.935768 |
| 855_14 | Peak_183 | 176 | 147 | 297.2215 | 3.87669 | 297.209 | 297.2309 | 3.831584 | 3.935768 |
| 855_14 | Peak_178 | 171 | 147 | 289.2151 | 3.874659 | 289.2041 | 289.2233 | 3.831584 | 3.935768 |
| 855_14 | Peak_281 | 261 | 147 | 403.2823 | 3.87619 | 403.2647 | 403.2959 | 3.831584 | 3.935768 |
| 855_14 | Peak_336 | 313 | 147 | 531.4031 | 3.876874 | 531.3828 | 531.4251 | 3.831584 | 3.92083 |
| 855_14 | Peak_326 | 304 | 147 | 505.4337 | 3.875306 | 505.4171 | 505.4583 | 3.831584 | 3.92083 |
| 855_14 | Peak_258 | 239 | 147 | 367.2952 | 3.877209 | 367.2788 | 367.3085 | 3.831584 | 3.935768 |
| 855_14 | Peak_310 | 288 | 147 | 459.3422 | 3.877432 | 459.3249 | 459.3521 | 3.831584 | 3.935768 |
| 855_14 | Peak_017 | 24 | 147 | 113.0962 | 3.876396 | 113.0891 | 113.1041 | 3.831584 | 3.935768 |
| 855_14 | Peak_058 | 65 | 147 | 183.1166 | 3.874747 | 183.1075 | 183.1266 | 3.831584 | 3.92083 |
| 855_14 | Peak_304 | 282 | 147 | 445.3287 | 3.877735 | 445.3119 | 445.3387 | 3.831584 | 3.935768 |
| 855_14 | Peak_105 | 106 | 147 | 235.2063 | 3.876962 | 235.1955 | 235.2172 | 3.846155 | 3.935768 |
| 855_14 | Peak_316 | 294 | 147 | 475.3771 | 3.874392 | 475.3597 | 475.3936 | 3.831584 | 3.92083 |
| 855_14 | Peak_055 | 62 | 147 | 177.1283 | 3.874794 | 177.1196 | 177.1384 | 3.831584 | 3.935768 |
| 855_14 | Peak_115 | 113 | 147 | 243.1749 | 3.876539 | 243.1654 | 243.183 | 3.846155 | 3.935768 |
| 855_14 | Peak_312 | 290 | 147 | 461.3619 | 3.876038 | 461.3449 | 461.3752 | 3.831584 | 3.92083 |
| 855_14 | Peak_068 | 73 | 147 | 193.1944 | 3.877035 | 193.1862 | 193.2038 | 3.831584 | 3.92083 |
| 855_14 | Peak_355 | 330 | 147 | 587.5024 | 3.876639 | 587.4804 | 587.5283 | 3.831584 | 3.935768 |
| 855_14 | Peak_015 | 22 | 147 | 111.0816 | 3.874781 | 111.0732 | 111.0896 | 3.831584 | 3.935768 |
| 855_14 | Peak_090 | 92 | 147 | 221.1902 | 3.874761 | 221.1798 | 221.2008 | 3.831584 | 3.935768 |
| 855_14 | Peak_064 | 69 | 147 | 189.1286 | 3.877384 | 189.1192 | 189.1386 | 3.831584 | 3.92083 |
| 855_14 | Peak_034 | 41 | 147 | 143.0858 | 3.876664 | 143.0782 | 143.0934 | 3.831584 | 3.935768 |
| 855_14 | Peak_077 | 81 | 147 | 207.1746 | 3.876297 | 207.1657 | 207.184 | 3.831584 | 3.92083 |
| 855_12 | Peak_340 | 317 | 147 | 542.4649 | 3.822245 | 542.4443 | 542.487 | 3.775677 | 3.890968 |
| 855_14 | Peak_219 | 210 | 147 | 335.2945 | 3.874825 | 335.2862 | 335.3069 | 3.831584 | 3.92083 |
| 855_14 | Peak_119 | 117 | 147 | 247.1703 | 3.877944 | 247.159 | 247.1812 | 3.846155 | 3.935768 |
| 855_13 | Peak_198 | 190 | 147 | 314.2768 | 3.847426 | 314.262 | 314.2945 | 3.789976 | 3.92083 |
| 855_14 | Peak_085 | 87 | 147 | 217.1585 | 3.875436 | 217.1488 | 217.1676 | 3.846155 | 3.935768 |
| 855_14 | Peak_259 | 240 | 147 | 369.3517 | 3.877215 | 369.3403 | 369.3674 | 3.846155 | 3.935768 |
| 855_14 | Peak_321 | 299 | 147 | 489.3928 | 3.876198 | 489.3688 | 489.4094 | 3.831584 | 3.92083 |
| 855_14 | Peak_325 | 303 | 147 | 503.4083 | 3.876342 | 503.3882 | 503.4357 | 3.831584 | 3.935768 |
| 855_14 | Peak_180 | 173 | 147 | 289.2881 | 3.877593 | 289.2809 | 289.3025 | 3.846155 | 3.935768 |
| 855_14 | Peak_237 | 224 | 147 | 349.2736 | 3.879035 | 349.2645 | 349.2856 | 3.846155 | 3.935768 |
| 855_14 | Peak_048 | 55 | 147 | 163.1131 | 3.876471 | 163.1032 | 163.123 | 3.846155 | 3.935768 |
| 855_14 | Peak_021 | 28 | 147 | 123.0812 | 3.877374 | 123.0737 | 123.0894 | 3.846155 | 3.935768 |
| 855_14 | Peak_131 | 129 | 147 | 261.1841 | 3.875343 | 261.1729 | 261.1911 | 3.831584 | 3.935768 |
| 855_14 | Peak_043 | 50 | 147 | 151.1127 | 3.877512 | 151.1047 | 151.122 | 3.831584 | 3.935768 |
| 855_14 | Peak_004 | 14 | 147 | 97.06637 | 3.873964 | 97.05898 | 97.07566 | 3.831584 | 3.935768 |
| 855_12 | Peak_332 | 309 | 147 | 524.4543 | 3.828382 | 524.4327 | 524.478 | 3.789976 | 3.905897 |
| 855_14 | Peak_175 | 168 | 147 | 287.2009 | 3.877837 | 287.1888 | 287.2103 | 3.846155 | 3.935768 |
| 855_14 | Peak_245 | 229 | 147 | 355.3351 | 3.877876 | 355.3236 | 355.3502 | 3.846155 | 3.935768 |
| 855_14 | Peak_274 | 255 | 147 | 397.3461 | 3.875111 | 397.3312 | 397.3594 | 3.831584 | 3.935768 |
| 855_14 | Peak_041 | 48 | 147 | 149.0971 | 3.878888 | 149.09 | 149.1055 | 3.831584 | 3.935768 |
| 855_14 | Peak_137 | 135 | 147 | 263.2733 | 3.879062 | 263.2666 | 263.2849 | 3.846155 | 3.935768 |
| 855_14 | Peak_317 | 295 | 147 | 477.3921 | 3.874925 | 477.3746 | 477.4116 | 3.831584 | 3.92083 |
| 855_14 | Peak_031 | 38 | 147 | 137.0973 | 3.876044 | 137.0893 | 137.1058 | 3.831584 | 3.935768 |
| 855_14 | Peak_209 | 200 | 147 | 327.3053 | 3.876869 | 327.2962 | 327.3192 | 3.846155 | 3.935768 |
| 855_12 | Peak_134 | 132 | 147 | 262.225 | 3.828788 | 262.2141 | 262.237 | 3.789976 | 3.92083 |
| 855_14 | Peak_302 | 280 | 147 | 441.4135 | 3.87669 | 441.4065 | 441.4272 | 3.831584 | 3.92083 |
| 855_14 | Peak_109 | 109 | 147 | 237.221 | 3.876259 | 237.2108 | 237.2304 | 3.831584 | 3.92083 |
| 855_14 | Peak_013 | 20 | 147 | 109.0653 | 3.871656 | 109.0578 | 109.0725 | 3.831584 | 3.92083 |
| 855_14 | Peak_051 | 58 | 147 | 169.1017 | 3.878278 | 169.0935 | 169.11 | 3.846155 | 3.935768 |
| 855_14 | Peak_026 | 33 | 147 | 129.0697 | 3.880395 | 129.0625 | 129.0786 | 3.846155 | 3.935768 |
| 855_14 | Peak_029 | 36 | 147 | 135.0811 | 3.876602 | 135.0722 | 135.0902 | 3.831584 | 3.92083 |
| 855_13 | Peak_352 | 327 | 147 | 576.5125 | 3.858611 | 576.4986 | 576.5325 | 3.805008 | 3.92083 |

| | | | | | | | | MS/MS library |
|---|---|---|---|---|---|---|---|---|
| UnitMassFile_R | Name | Library. m.z | Library. RT | Slope | RSQ | UnitMass_File | R ClusterNumber | annotation |
| 855_14 | Peak_358 | 599.5022 | 3.86 | 308.7462 | 0.935554 | 855 | 14 | TAG |
| | | | | | | | | (C16:0, C18:1, C18:3) |
| | | | | | | | | (+H) Fragment 1 |
| 855_14 | Peak_341 | 543.4759 | 3.86 | 105.4506 | 0.945741 | 855 | 14 | TAG |
| | | | | | | | | (C16:0, C18:1, C18:3) |
| | | | | | | | | (+H) Fragment 3 |
| 855_14 | Peak_333 | 525.4656 | 3.86 | 82.62495 | 0.952203 | 855 | 14 | TAG |
| | | | | | | | | (C16:0, C18:1, C18:3) |
| | | | | | | | | (+H) Fragment 4 |
| 855_14 | Peak_384 | 855.7427 | 3.86 | 79.62756 | 0.927787 | 855 | 14 | TAG |
| | | | | | | | | (C16:0, C18:1, C18:3) |
| | | | | | | | | (+H) Fragment 2 |
| 855_14 | Peak_197 | 313.2736 | 3.86 | 65.42283 | 0.912198 | 855 | 14 | TAG |
| | | | | | | | | (C16:0, C18:1, C18:3) |
| | | | | | | | | (+H) Fragment 5 |
| 855_14 | Peak_003 | | | 57.44355 | 0.917356 | 855 | 14 | |
| 855_14 | Peak_132 | 261.221 | 3.86 | 47.23602 | 0.891414 | 855 | 14 | TAG |
| | | | | | | | | (C16:0, C18:1, C18:3) |
| | | | | | | | | (+H) Fragment 7 |
| 855_14 | Peak_014 | | | 36.00052 | 0.90172 | 855 | 14 | |
| 855_14 | Peak_359 | 600.5042 | 3.86 | 31.4391 | 0.613952 | 855 | 14 | TAG |
| | | | | | | | | (C16:0, C18:1, C18:3) |
| | | | | | | | | (+H) Fragment 6 |
| 855_14 | Peak_020 | | | 24.22542 | 0.937424 | 855 | 14 | |
| 855_14 | Peak_353 | 577.5177 | 3.86 | 23.88388 | 0.933695 | 855 | 14 | TAG |
| | | | | | | | | (C16:0, C18:1, C18:3) |
| | | | | | | | | (+H) Fragment 8 |
| 855_14 | Peak_022 | | | 20.72728 | 0.92705 | 855 | 14 | |
| 855_14 | Peak_030 | | | 20.5318 | 0.910393 | 855 | 14 | |
| 855_14 | Peak_116 | | | 19.69703 | 0.902677 | 855 | 14 | |
| 855_14 | Peak_042 | | | 17.00445 | 0.905145 | 855 | 14 | |
| 855_14 | Peak_005 | | | 16.18922 | 0.888037 | 855 | 14 | |
| 855_14 | Peak_390 | | | 15.2301 | 0.638552 | 855 | 14 | |
| 855_14 | Peak_054 | | | 15.1287 | 0.920858 | 855 | 14 | |
| 855_14 | Peak_002 | | | 15.11405 | 0.892878 | 855 | 14 | |
| 855_14 | Peak_201 | | | 14.54589 | 0.893316 | 855 | 14 | |
| 855_14 | Peak_362 | | | 14.47472 | 0.889371 | 855 | 14 | |
| 855_14 | Peak_063 | | | 14.39914 | 0.896294 | 855 | 14 | |
| 855_14 | Peak_012 | | | 13.83073 | 0.889621 | 855 | 14 | |
| 855_14 | Peak_112 | | | 13.76019 | 0.868384 | 855 | 14 | |
| 855_14 | Peak_053 | | | 13.6341 | 0.871246 | 855 | 14 | |
| 855_14 | Peak_028 | | | 13.44522 | 0.918049 | 855 | 14 | |
| 855_14 | Peak_019 | | | 13.3345 | 0.893005 | 855 | 14 | |
| 855_14 | Peak_351 | | | 13.30051 | 0.956078 | 855 | 14 | |
| 855_14 | Peak_179 | | | 12.95796 | 0.869716 | 855 | 14 | |
| 855_14 | Peak_159 | | | 12.56948 | 0.892854 | 855 | 14 | |
| 855_14 | Peak_289 | | | 12.41179 | 0.873168 | 855 | 14 | |
| 855_14 | Peak_032 | | | 12.36107 | 0.934046 | 855 | 14 | |
| 855_14 | Peak_349 | | | 12.15945 | 0.909443 | 855 | 14 | |
| 855_14 | Peak_327 | | | 12.14752 | 0.884913 | 855 | 14 | |
| 855_14 | Peak_065 | | | 11.95936 | 0.821077 | 855 | 14 | |
| 855_14 | Peak_072 | | | 11.8693 | 0.936636 | 855 | 14 | |
| 855_14 | Peak_047 | | | 11.64905 | 0.887194 | 855 | 14 | |
| 855_14 | Peak_049 | | | 11.62582 | 0.868705 | 855 | 14 | |
| 855_14 | Peak_271 | | | 11.47282 | 0.931679 | 855 | 14 | |
| 855_14 | Peak_035 | | | 11.45196 | 0.910063 | 855 | 14 | |
| 855_14 | Peak_263 | | | 11.32693 | 0.932733 | 855 | 14 | |
| 855_14 | Peak_190 | | | 11.32649 | 0.91014 | 855 | 14 | |
| 855_14 | Peak_038 | | | 11.0009 | 0.941687 | 855 | 14 | |
| 855_14 | Peak_354 | | | 10.05769 | 0.875646 | 855 | 14 | |
| 855_14 | Peak_361 | | | 10.03252 | 0.935513 | 855 | 14 | |
| 855_14 | Peak_373 | | | 9.834581 | 0.837843 | 855 | 14 | |
| 855_14 | Peak_084 | | | 9.822833 | 0.79202 | 855 | 14 | |
| 855_14 | Peak_276 | | | 9.644689 | 0.870362 | 855 | 14 | |
| 855_14 | Peak_073 | | | 9.208077 | 0.940849 | 855 | 14 | |
| 855_14 | Peak_027 | | | 9.185525 | 0.87457 | 855 | 14 | |
| 855_14 | Peak_056 | | | 9.125911 | 0.914357 | 855 | 14 | |
| 855_14 | Peak_046 | | | 9.042069 | 0.898879 | 855 | 14 | |
| 855_14 | Peak_328 | | | 8.895123 | 0.896615 | 855 | 14 | |
| 855_14 | Peak_011 | | | 8.838666 | 0.8397 | 855 | 14 | |
| 855_14 | Peak_253 | | | 8.677882 | 0.876481 | 855 | 14 | |
| 855_14 | Peak_222 | | | 8.536661 | 0.888997 | 855 | 14 | |
| 855_14 | Peak_121 | | | 8.092048 | 0.833363 | 855 | 14 | |
| 855_14 | Peak_282 | | | 7.998619 | 0.904767 | 855 | 14 | |
| 855_14 | Peak_324 | | | 7.86757 | 0.930778 | 855 | 14 | |
| 855_14 | Peak_285 | | | 7.853292 | 0.88544 | 855 | 14 | |
| 855_14 | Peak_279 | | | 7.698896 | 0.910304 | 855 | 14 | |
| 855_14 | Peak_136 | | | 7.547979 | 0.860465 | 855 | 14 | |
| 855_14 | Peak_103 | | | 7.511265 | 0.868483 | 855 | 14 | |
| 855_14 | Peak_100 | | | 7.197898 | 0.895928 | 855 | 14 | |
| 855_14 | Peak_044 | | | 7.155647 | 0.866447 | 855 | 14 | |
| 855_14 | Peak_118 | | | 7.153107 | 0.824103 | 855 | 14 | |
| 855_14 | Peak_311 | | | 6.982977 | 0.916048 | 855 | 14 | |
| 855_14 | Peak_233 | | | 6.92873 | 0.810695 | 855 | 14 | |
| 855_14 | Peak_067 | | | 6.765274 | 0.861507 | 855 | 14 | |
| 855_14 | Peak_062 | | | 6.751024 | 0.85094 | 855 | 14 | |
| 855_14 | Peak_303 | | | 6.705316 | 0.898094 | 855 | 14 | |
| 855_14 | Peak_016 | | | 6.657985 | 0.892732 | 855 | 14 | |
| 855_14 | Peak_294 | | | 6.593872 | 0.919904 | 855 | 14 | |
| 855_14 | Peak_202 | | | 6.567895 | 0.757199 | 855 | 14 | |
| 855_14 | Peak_298 | | | 6.542326 | 0.896625 | 855 | 14 | |
| 855_14 | Peak_204 | | | 6.53584 | 0.792402 | 855 | 14 | |
| 855_14 | Peak_070 | | | 6.532963 | 0.90044 | 855 | 14 | |
| 855_14 | Peak_346 | | | 6.502347 | 0.850965 | 855 | 14 | |
| 855_14 | Peak_214 | | | 6.281897 | 0.839521 | 855 | 14 | |
| 855_14 | Peak_305 | | | 6.261065 | 0.840707 | 855 | 14 | |
| 855_14 | Peak_191 | | | 6.160772 | 0.865705 | 855 | 14 | |
| 855_14 | Peak_086 | | | 6.135495 | 0.941002 | 855 | 14 | |
| 855_14 | Peak_315 | | | 5.904899 | 0.873508 | 855 | 14 | |
| 855_14 | Peak_292 | | | 5.674221 | 0.866284 | 855 | 14 | |
| 855_14 | Peak_127 | | | 5.639685 | 0.916397 | 855 | 14 | |
| 855_14 | Peak_217 | | | 5.634883 | 0.875394 | 855 | 14 | |
| 855_14 | Peak_075 | | | 5.621665 | 0.898553 | 855 | 14 | |
| 855_14 | Peak_301 | | | 5.482182 | 0.834654 | 855 | 14 | |
| 855_14 | Peak_091 | | | 5.342212 | 0.862502 | 855 | 14 | |
| 855_14 | Peak_083 | | | 5.318366 | 0.893934 | 855 | 14 | |
| 855_14 | Peak_342 | | | 5.302219 | 0.589792 | 855 | 14 | |
| 855_14 | Peak_181 | | | 5.296974 | 0.904772 | 855 | 14 | |
| 855_14 | Peak_200 | | | 5.168305 | 0.856959 | 855 | 14 | |
| 855_14 | Peak_319 | | | 5.107229 | 0.876243 | 855 | 14 | |
| 855_14 | Peak_052 | | | 5.106138 | 0.858732 | 855 | 14 | |
| 855_14 | Peak_101 | | | 5.075797 | 0.873653 | 855 | 14 | |
| 855_14 | Peak_088 | | | 5.02592 | 0.837952 | 855 | 14 | |
| 855_14 | Peak_286 | | | 4.885013 | 0.723115 | 855 | 14 | |
| 855_14 | Peak_252 | | | 4.817051 | 0.713593 | 855 | 14 | |
| 855_14 | Peak_348 | | | 4.8066 | 0.8341 | 855 | 14 | |
| 855_14 | Peak_234 | | | 4.711962 | 0.856043 | 855 | 14 | |
| 855_14 | Peak_187 | | | 4.701269 | 0.879583 | 855 | 14 | |
| 855_14 | Peak_269 | | | 4.627359 | 0.842044 | 855 | 14 | |
| 855_14 | Peak_272 | | | 4.561071 | 0.810496 | 855 | 14 | |
| 855_14 | Peak_161 | | | 4.560836 | 0.866164 | 855 | 14 | |
| 855_14 | Peak_360 | | | 4.555802 | 0.860467 | 855 | 14 | |
| 855_14 | Peak_176 | | | 4.53355 | 0.831351 | 855 | 14 | |
| 855_14 | Peak_335 | | | 4.476427 | 0.877502 | 855 | 14 | |
| 855_14 | Peak_221 | | | 4.468974 | 0.843603 | 855 | 14 | |
| 855_14 | Peak_213 | | | 4.467811 | 0.918245 | 855 | 14 | |
| 855_14 | Peak_215 | | | 4.460119 | 0.880499 | 855 | 14 | |
| 855_14 | Peak_099 | | | 4.391549 | 0.846844 | 855 | 14 | |
| 855_14 | Peak_130 | | | 4.384048 | 0.825699 | 855 | 14 | |
| 855_14 | Peak_157 | | | 4.370834 | 0.889571 | 855 | 14 | |
| 855_14 | Peak_050 | | | 4.345549 | 0.855596 | 855 | 14 | |
| 855_14 | Peak_001 | | | 4.255699 | 0.832813 | 855 | 14 | |
| 855_14 | Peak_114 | | | 4.217421 | 0.769764 | 855 | 14 | |
| 855_14 | Peak_185 | | | 4.209303 | 0.745436 | 855 | 14 | |
| 855_14 | Peak_154 | | | 4.091047 | 0.787865 | 855 | 14 | |
| 855_14 | Peak_297 | | | 4.012747 | 0.807394 | 855 | 14 | |
| 855_14 | Peak_367 | | | 3.995424 | 0.934425 | 855 | 14 | |
| 855_14 | Peak_308 | | | 3.925446 | 0.812426 | 855 | 14 | |
| 855_14 | Peak_236 | | | 3.895122 | 0.725974 | 855 | 14 | |
| 855_14 | Peak_293 | | | 3.859533 | 0.859196 | 855 | 14 | |
| 855_14 | Peak_208 | | | 3.822611 | 0.903898 | 855 | 14 | |
| 855_14 | Peak_150 | | | 3.806458 | 0.84478 | 855 | 14 | |
| 855_14 | Peak_369 | | | 3.749511 | 0.78802 | 855 | 14 | |
| 855_14 | Peak_339 | | | 3.739274 | 0.839658 | 855 | 14 | |
| 855_14 | Peak_334 | | | 3.721296 | 0.609175 | 855 | 14 | |
| 855_14 | Peak_307 | | | 3.716238 | 0.91859 | 855 | 14 | |
| 855_14 | Peak_120 | | | 3.69808 | 0.702027 | 855 | 14 | |
| 855_14 | Peak_265 | | | 3.696775 | 0.835853 | 855 | 14 | |
| 855_14 | Peak_140 | | | 3.662351 | 0.88476 | 855 | 14 | |
| 855_14 | Peak_229 | | | 3.626099 | 0.837984 | 855 | 14 | |
| 855_14 | Peak_309 | | | 3.615921 | 0.836325 | 855 | 14 | |
| 855_14 | Peak_133 | | | 3.600688 | 0.73747 | 855 | 14 | |
| 855_14 | Peak_129 | | | 3.537284 | 0.74933 | 855 | 14 | |
| 855_14 | Peak_320 | | | 3.456469 | 0.876247 | 855 | 14 | |
| 855_14 | Peak_106 | | | 3.431652 | 0.84397 | 855 | 14 | |
| 855_14 | Peak_331 | | | 3.427213 | 0.852814 | 855 | 14 | |
| 855_14 | Peak_156 | | | 3.396288 | 0.777707 | 855 | 14 | |
| 855_14 | Peak_125 | | | 3.377164 | 0.730112 | 855 | 14 | |
| 855_14 | Peak_278 | | | 3.318767 | 0.752397 | 855 | 14 | |
| 855_14 | Peak_300 | | | 3.300311 | 0.923733 | 855 | 14 | |
| 855_14 | Peak_173 | | | 3.288624 | 0.784237 | 855 | 14 | |
| 855_14 | Peak_261 | | | 3.27625 | 0.746684 | 855 | 14 | |
| 855_14 | Peak_248 | | | 3.234473 | 0.837985 | 855 | 14 | |
| 855_14 | Peak_174 | | | 3.220992 | 0.85107 | 855 | 14 | |
| 855_I4 | Peak_212 | | | 3.193644 | 0.779592 | 855 | 14 | |
| 855_14 | Peak_260 | | | 3.167646 | 0.811883 | 855 | 14 | |
| 855_14 | Peak_225 | | | 3.160002 | 0.865975 | 855 | 14 | |
| 855_14 | Peak_343 | | | 3.150723 | 0.846626 | 855 | 14 | |
| 855_14 | Peak_284 | | | 3.147334 | 0.725974 | 855 | 14 | |
| 855_14 | Peak_182 | | | 3.14449 | 0.82957 | 855 | 14 | |
| 855_14 | Peak_262 | | | 3.120208 | 0.643026 | 855 | 14 | |
| 855_14 | Peak_256 | | | 3.100609 | 0.817221 | 855 | 14 | |
| 855_14 | Peak_135 | | | 3.096829 | 0.783564 | 855 | 14 | |
| 855_14 | Peak_128 | | | 3.093843 | 0.847085 | 855 | 14 | |
| 855_14 | Peak_193 | | | 3.070559 | 0.66048 | 855 | 14 | |
| 855_14 | Peak_057 | | | 3.025177 | 0.77108 | 855 | 14 | |
| 855_14 | Peak_264 | | | 3.005177 | 0.673739 | 855 | 14 | |
| 855_14 | Peak_102 | | | 3.001177 | 0.837367 | 855 | 14 | |
| 855_14 | Peak_275 | | | 2.996054 | 0.744502 | 855 | 14 | |
| 855_14 | Peak_169 | | | 2.992167 | 0.806814 | 855 | 14 | |
| 855_14 | Peak_314 | | | 2.984057 | 0.740242 | 855 | 14 | |
| 855_14 | Peak_283 | | | 2.96351 | 0.808977 | 855 | 14 | |
| 855_14 | Peak_313 | | | 2.898935 | 0.796365 | 855 | 14 | |
| 855_14 | Peak_249 | | | 2.881459 | 0.888248 | 855 | 14 | |
| 855_14 | Peak_160 | | | 2.87908 | 0.775782 | 855 | 14 | |
| 855_14 | Peak_268 | | | 2.871338 | 0.774926 | 855 | 14 | |
| 855_14 | Peak_206 | | | 2.867228 | 0.736662 | 855 | 14 | |
| 855_14 | Peak_199 | | | 2.840456 | 0.835152 | 855 | 14 | |
| 855_14 | Peak_177 | | | 2.84003 | 0.772872 | 855 | 14 | |
| 855_12 | Peak_357 | | | 2.839915 | 0.837888 | 855 | 12 | |
| 855_14 | Peak_069 | | | 2.81493 | 0.726175 | 855 | 14 | |
| 855_14 | Peak_230 | | | 2.789903 | 0.861454 | 855 | 14 | |
| 855_14 | Peak_163 | | | 2.783823 | 0.887532 | 855 | 14 | |
| 855_14 | Peak_220 | | | 2.770969 | 0.858717 | 855 | 14 | |
| 855_14 | Peak_368 | | | 2.761618 | 0.737326 | 855 | 14 | |
| 855_14 | Peak_287 | | | 2.76161 | 0.881594 | 855 | 14 | |
| 855_14 | Peak_218 | | | 2.752723 | 0.821182 | 855 | 14 | |
| 855_14 | Peak_192 | | | 2.731459 | 0.804266 | 855 | 14 | |
| 855_14 | Peak_117 | | | 2.708726 | 0.772137 | 855 | 14 | |
| 855_14 | Peak_033 | | | 2.706491 | 0.811208 | 855 | 14 | |
| 855_14 | Peak_370 | | | 2.690555 | 0.78736 | 855 | 14 | |
| 855_14 | Peak_097 | | | 2.689479 | 0.779748 | 855 | 14 | |
| 855_14 | Peak_244 | | | 2.683134 | 0.859257 | 855 | 14 | |
| 855_14 | Peak_270 | | | 2.678621 | 0.690709 | 855 | 14 | |
| 855_14 | Peak_322 | | | 2.677549 | 0.914851 | 855 | 14 | |
| 855_14 | Peak_158 | | | 2.663702 | 0.685142 | 855 | 14 | |
| 855_14 | Peak_255 | | | 2.661492 | 0.809114 | 855 | 14 | |
| 855_14 | Peak_024 | | | 2.658178 | 0.840486 | 855 | 14 | |
| 855_14 | Peak_267 | | | 2.650632 | 0.8764 | 855 | 14 | |
| 855_14 | Peak_153 | | | 2.61123 | 0.825708 | 855 | 14 | |
| 855_14 | Peak_216 | | | 2.60337 | 0.746626 | 855 | 14 | |
| 855_14 | Peak_018 | | | 2.595306 | 0.784307 | 855 | 14 | |
| 855_14 | Peak_189 | | | 2.588062 | 0.67909 | 855 | 14 | |
| 855_14 | Peak_087 | | | 2.565476 | 0.828418 | 855 | 14 | |
| 855_14 | Peak_290 | | | 2.543123 | 0.727062 | 855 | 14 | |
| 855_14 | Peak_318 | | | 2.539348 | 0.760347 | 855 | 14 | |
| 855_14 | Peak_306 | | | 2.53849 | 0.806634 | 855 | 14 | |
| 855_14 | Peak_123 | | | 2.533582 | 0.860504 | 855 | 14 | |
| 855_14 | Peak_273 | | | 2.530734 | 0.779624 | 855 | 14 | |
| 855_14 | Peak_082 | | | 2.519347 | 0.864424 | 855 | 14 | |
| 855_14 | Peak_122 | | | 2.51296 | 0.71797 | 855 | 14 | |
| 855_14 | Peak_074 | | | 2.510944 | 0.795026 | 855 | 14 | |
| 855_14 | Peak_241 | | | 2.478295 | 0.822602 | 855 | 14 | |
| 855_14 | Peak_111 | | | 2.43629 | 0.812031 | 855 | 14 | |
| 855_14 | Peak_124 | | | 2.419955 | 0.870618 | 855 | 14 | |
| 855_14 | Peak_205 | | | 2.374005 | 0.775273 | 855 | 14 | |
| 855_14 | Peak_025 | | | 2.358237 | 0.775696 | 855 | 14 | |
| 855_14 | Peak_126 | | | 2.352665 | 0.842192 | 855 | 14 | |
| 855_14 | Peak_323 | | | 2.334253 | 0.72155 | 855 | 14 | |
| 855_14 | Peak_337 | | | 2.313788 | 0.750281 | 855 | 14 | |
| 855_14 | Peak_045 | | | 2.290158 | 0.774753 | 855 | 14 | |
| 855_14 | Peak_104 | | | 2.277736 | 0.842295 | 855 | 14 | |
| 855_14 | Peak_162 | | | 2.274849 | 0.806001 | 855 | 14 | |
| 855_14 | Peak_371 | | | 2.250873 | 0.794496 | 855 | 14 | |
| 855_14 | Peak_184 | | | 2.242832 | 0.805345 | 855 | 14 | |
| 855_14 | Peak_291 | | | 2.234534 | 0.752731 | 855 | 14 | |
| 855_14 | Peak_139 | | | 2.20683 | 0.735187 | 855 | 14 | |
| 855_14 | Peak_186 | | | 2.167112 | 0.767822 | 855 | 14 | |
| 855_14 | Peak_007 | | | 2.124929 | 0.74444 | 855 | 14 | |
| 855_14 | Peak_356 | | | 2.117797 | 0.718066 | 855 | 14 | |
| 855_14 | Peak_296 | | | 2.105648 | 0.689889 | 855 | 14 | |
| 855_14 | Peak_194 | | | 2.105028 | 0.778367 | 855 | 14 | |
| 855_14 | Peak_078 | | | 2.08341 | 0.841742 | 855 | 14 | |
| 855_14 | Peak_066 | | | 2.076132 | 0.809127 | 855 | 14 | |
| 855_14 | Peak_288 | | | 2.065356 | 0.682706 | 855 | 14 | |
| 855_14 | Peak_006 | | | 2.058018 | 0.789653 | 855 | 14 | |
| 855_I4 | Peak_366 | | | 2.057735 | 0.662755 | 855 | 14 | |
| 855_14 | Peak_347 | | | 2.056489 | 0.802265 | 855 | 14 | |
| 855_14 | Peak_188 | | | 2.051732 | 0.733463 | 855 | 14 | |
| 855_14 | Peak_183 | | | 2.051014 | 0.706258 | 855 | 14 | |
| 855_14 | Peak_178 | | | 2.042051 | 0.626395 | 855 | 14 | |
| 855_14 | Peak_281 | | | 1.985039 | 0.724371 | 855 | 14 | |
| 855_14 | Peak_336 | | | 1.948082 | 0.735299 | 855 | 14 | |
| 855_14 | Peak_326 | | | 1.946853 | 0.690959 | 855 | 14 | |
| 855_14 | Peak_258 | | | 1.9444 | 0.605776 | 855 | 14 | |
| 855_14 | Peak_310 | | | 1.902557 | 0.525197 | 855 | 14 | |
| 855_14 | Peak_017 | | | 1.882401 | 0.81789 | 855 | 14 | |
| 855_14 | Peak_058 | | | 1.881063 | 0.710378 | 855 | 14 | |
| 855_14 | Peak_304 | | | 1.873385 | 0.732798 | 855 | 14 | |
| 855_14 | Peak_105 | | | 1.835633 | 0.652532 | 855 | 14 | |
| 855_14 | Peak_316 | | | 1.829971 | 0.783019 | 855 | 14 | |
| 855_14 | Peak_055 | | | 1.825538 | 0.689041 | 855 | 14 | |
| 855_14 | Peak_115 | | | 1.723699 | 0.766113 | 855 | 14 | |
| 855_14 | Peak_312 | | | 1.709058 | 0.650414 | 855 | 14 | |
| 855_14 | Peak_068 | | | 1.698422 | 0.857371 | 855 | 14 | |
| 855_14 | Peak_355 | | | 1.672885 | 0.862197 | 855 | 14 | |
| 855_14 | Peak_015 | | | 1.671951 | 0.661797 | 855 | 14 | |
| 855_14 | Peak_090 | | | 1.666767 | 0.712546 | 855 | 14 | |
| 855_14 | Peak_064 | | | 1.654582 | 0.634506 | 855 | 14 | |
| 855_14 | Peak_034 | | | 1.651748 | 0.800996 | 855 | 14 | |
| 855_14 | Peak_077 | | | 1.608937 | 0.754283 | 855 | 14 | |
| 855_12 | Peak_340 | | | 1.603724 | 0.634189 | 855 | 12 | |
| 855_14 | Peak_219 | | | 1.601846 | 0.71178 | 855 | 14 | |
| 855_14 | Peak_119 | | | 1.595145 | 0.832526 | 855 | 14 | |
| 855_13 | Peak_198 | | | 1.556905 | 0.7983 | 855 | 13 | |
| 855_14 | Peak_085 | | | 1.540921 | 0.657296 | 855 | 14 | |
| 855_14 | Peak_259 | | | 1.538289 | 0.746578 | 855 | 14 | |
| 855_14 | Peak_321 | | | 1.535915 | 0.652051 | 855 | 14 | |
| 855_14 | Peak_325 | | | 1.531122 | 0.77983 | 855 | 14 | |
| 855_14 | Peak_180 | | | 1.505953 | 0.724498 | 855 | 14 | |
| 855_14 | Peak_237 | | | 1.505121 | 0.747507 | 855 | 14 | |
| 855_14 | Peak_048 | | | 1.479777 | 0.67348 | 855 | 14 | |
| 855_14 | Peak_021 | | | 1.463426 | 0.598036 | 855 | 14 | |
| 855_14 | Peak_131 | | | 1.454819 | 0.398798 | 855 | 14 | |
| 855_14 | Peak_043 | | | 1.435095 | 0.762846 | 855 | 14 | |
| 855_14 | Peak_004 | | | 1.41941 | 0.561593 | 855 | 14 | |
| 855_12 | Peak_332 | | | 1.419162 | 0.605558 | 855 | 12 | |
| 855_14 | Peak_175 | | | 1.402216 | 0.674731 | 855 | 14 | |
| 855_14 | Peak_245 | | | 1.346859 | 0.715181 | 855 | 14 | |
| 855_14 | Peak_274 | | | 1.343683 | 0.784134 | 855 | 14 | |
| 855_14 | Peak_041 | | | 1.342408 | 0.81634 | 855 | 14 | |
| 855_14 | Peak_137 | | | 1.310246 | 0.616473 | 855 | 14 | |
| 855_14 | Peak_317 | | | 1.295732 | 0.811852 | 855 | 14 | |
| 855_14 | Peak_031 | | | 1.286448 | 0.527785 | 855 | 14 | |
| 855_14 | Peak_209 | | | 1.275354 | 0.618621 | 855 | 14 | |
| 855_12 | Peak_134 | | | 1.24066 | 0.585717 | 855 | 12 | |
| 855_14 | Peak_302 | | | 1.180402 | 0.572597 | 855 | 14 | |
| 855_14 | Peak_109 | | | 1.157108 | 0.540362 | 855 | 14 | |
| 855_14 | Peak_013 | | | 1.134654 | 0.507961 | 855 | 14 | |
| 855_14 | Peak_051 | | | 1.090276 | 0.534443 | 855 | 14 | |
| 855_14 | Peak_026 | | | 0.917275 | 0.58559 | 855 | 14 | |
| 855_14 | Peak_029 | | | 0.892859 | 0.700174 | 855 | 14 | |
| 855_13 | Peak_352 | | | 0.826025 | 0.561526 | 855 | 13 | |

TABLE 3
Group of different fragment ions from precursor m/z 856.7459 corresponding
to ionized TAG (C16:0; C18:1; C18:3) found in the SWATH ® window m/z 856

| UnitMassFile_R | Name | Cluster | Group_FSD | m.z | RT | m.z. Min | m.z. Max | RT. Min | RT. Max |
|---|---|---|---|---|---|---|---|---|---|
| 856_12 | Peak_34342 | 1661 | 147 | 856.7459 | 3.853262 | 856.7209 | 856.7952 | 3.760183 | 3.9435 |
| 856_12 | Peak_394 | 307 | 147 | 600.507 | 3.866233 | 600.4633 | 600.5567 | 3.777335 | 3.945119 |
| 856_12 | Peak_393 | 306 | 147 | 599.5035 | 3.866549 | 599.4606 | 599.5608 | 3.791624 | 3.930708 |
| 856_12 | Peak_418 | 325 | 147 | 856.7472 | 3.865628 | 856.6942 | 856.8181 | 3.791624 | 3.930708 |
| 856_12 | Peak_004 | 3 | 147 | 95.0867 | 3.865621 | 95.07782 | 95.09847 | 3.791624 | 3.945119 |
| 856_12 | Peak_377 | 291 | 147 | 544.4802 | 3.865478 | 544.4383 | 544.524 | 3.791624 | 3.930708 |
| 856_12 | Peak_371 | 285 | 147 | 526.4701 | 3.866711 | 526.4356 | 526.5133 | 3.791624 | 3.930708 |
| 856_12 | Peak_220 | 177 | 147 | 313.274 | 3.86649 | 313.2444 | 313.3043 | 3.791624 | 3.945119 |
| 856_12 | Peak_376 | 290 | 147 | 543.4771 | 3.865572 | 543.4343 | 543.5231 | 3.791624 | 3.930708 |
| 856_12 | Peak_150 | 133 | 147 | 261.2217 | 3.866566 | 261.2048 | 261.2391 | 3.791624 | 3.930708 |
| 856_12 | Peak_020 | 7 | 147 | 109.1018 | 3.865586 | 109.0931 | 109.1123 | 3.80249 | 3.930708 |
| 856_12 | Peak_370 | 284 | 147 | 525.467 | 3.86507 | 525.4321 | 525.5129 | 3.791624 | 3.930708 |
| 856_12 | Peak_029 | 9 | 147 | 121.1015 | 3.866024 | 121.0915 | 121.1132 | 3.80249 | 3.930708 |
| 856_12 | Peak_221 | 178 | 147 | 314.2771 | 3.866596 | 314.242 | 314.3045 | 3.80249 | 3.930708 |
| 856_12 | Peak_042 | 13 | 147 | 135.1171 | 3.865197 | 135.1066 | 135.1296 | 3.80249 | 3.930708 |
| 856_12 | Peak_032 | 10 | 147 | 123.1171 | 3.865781 | 123.1082 | 123.1286 | 3.80249 | 3.930708 |
| 856_12 | Peak_007 | 4 | 147 | 97.10197 | 3.865957 | 97.09375 | 97.11183 | 3.80249 | 3.930708 |
| 856_12 | Peak_053 | 16 | 147 | 149.1326 | 3.865799 | 149.1211 | 149.1452 | 3.80249 | 3.930708 |
| 856_12 | Peak_002 | 2 | 147 | 93.07095 | 3.865486 | 93.06262 | 93.08032 | 3.80249 | 3.930708 |
| 856_12 | Peak_133 | 120 | 147 | 243.211 | 3.866452 | 243.1962 | 243.2314 | 3.80249 | 3.930708 |
| 856_12 | Peak_027 | 8 | 147 | 119.0859 | 3.8657 | 119.0776 | 119.096 | 3.80249 | 3.930708 |
| 856_12 | Peak_017 | 6 | 147 | 107.0861 | 3.865605 | 107.0783 | 107.0958 | 3.813356 | 3.930708 |
| 856_12 | Peak_389 | 303 | 147 | 578.5214 | 3.867455 | 578.4796 | 578.5611 | 3.813356 | 3.930708 |
| 856_12 | Peak_395 | 308 | 147 | 601.5091 | 3.869178 | 601.4737 | 601.5464 | 3.827645 | 3.930708 |
| 856_12 | Peak_069 | 21 | 147 | 173.1325 | 3.866133 | 173.1204 | 173.1464 | 3.80249 | 3.930708 |
| 856_12 | Peak_153 | 135 | 147 | 262.225 | 3.866626 | 262.2073 | 262.2416 | 3.80249 | 3.930708 |
| 856_12 | Peak_071 | 22 | 147 | 175.148 | 3.866333 | 175.1358 | 175.1619 | 3.80249 | 3.930708 |
| 856_12 | Peak_040 | 12 | 147 | 133.1013 | 3.866256 | 133.0912 | 133.1124 | 3.80249 | 3.930708 |
| 856_12 | Peak_082 | 24 | 147 | 187.1481 | 3.866481 | 187.1347 | 187.1637 | 3.80249 | 3.930708 |
| 856_12 | Peak_047 | 14 | 147 | 145.1011 | 3.865817 | 145.0911 | 145.1132 | 3.813356 | 3.930708 |
| 856_12 | Peak_084 | 25 | 147 | 189.1638 | 3.866259 | 189.1503 | 189.1774 | 3.813356 | 3.930708 |
| 856_12 | Peak_128 | 34 | 147 | 239.237 | 3.867653 | 239.2217 | 239.2544 | 3.813356 | 3.930708 |
| 856_12 | Peak_225 | 180 | 147 | 317.2471 | 3.86617 | 317.2241 | 317.2618 | 3.80249 | 3.930708 |
| 856_12 | Peak_064 | 19 | 147 | 163.148 | 3.866135 | 163.1375 | 163.1609 | 3.813356 | 3.930708 |
| 856_12 | Peak_050 | 15 | 147 | 147.117 | 3.86612 | 147.1077 | 147.1283 | 3.80249 | 3.930708 |
| 856_12 | Peak_045 | 79 | 147 | 137.1326 | 3.866024 | 137.124 | 137.1438 | 3.813356 | 3.930708 |
| 856_12 | Peak_038 | 11 | 147 | 131.0856 | 3.865803 | 131.0764 | 131.0974 | 3.813356 | 3.930708 |
| 856_12 | Peak_176 | 40 | 147 | 275.2372 | 3.866171 | 275.2241 | 275.2522 | 3.80249 | 3.930708 |
| 856_12 | Peak_094 | 27 | 147 | 201.1639 | 3.866864 | 201.1516 | 201.1776 | 3.813356 | 3.930708 |
| 856_12 | Peak_197 | 43 | 147 | 289.2526 | 3.866441 | 289.2401 | 289.2689 | 3.80249 | 3.930708 |
| 856_12 | Peak_061 | 18 | 147 | 161.1324 | 3.866219 | 161.1227 | 161.1442 | 3.813356 | 3.930708 |
| 856_12 | Peak_388 | 302 | 147 | 577.5186 | 3.867521 | 577.4785 | 577.5565 | 3.813356 | 3.930708 |
| 856_12 | Peak_059 | 17 | 147 | 159.1167 | 3.866876 | 159.1062 | 159.1293 | 3.80249 | 3.930708 |
| 856_12 | Peak_015 | 5 | 147 | 105.0705 | 3.866404 | 105.062 | 105.0793 | 3.80249 | 3.930708 |
| 856_12 | Peak_400 | 313 | 147 | 618.516 | 3.866154 | 618.4753 | 618.563 | 3.813356 | 3.930708 |
| 856_12 | Peak_209 | 171 | 147 | 303.2686 | 3.866441 | 303.2549 | 303.2869 | 3.813356 | 3.930708 |
| 856_12 | Peak_097 | 28 | 147 | 203.1792 | 3.867188 | 203.1665 | 203.1927 | 3.813356 | 3.930708 |
| 856_12 | Peak_074 | 23 | 147 | 177.1637 | 3.867025 | 177.1534 | 177.1759 | 3.813356 | 3.930708 |
| 856_12 | Peak_411 | 320 | 147 | 838.7344 | 3.866013 | 838.6816 | 838.7838 | 3.813356 | 3.930708 |
| 856_12 | Peak_105 | 30 | 147 | 215.1792 | 3.866803 | 215.167 | 215.1939 | 3.813356 | 3.930708 |
| 856_12 | Peak_364 | 279 | 147 | 508.457 | 3.866387 | 508.4267 | 508.4872 | 3.813356 | 3.930708 |
| 856_12 | Peak_323 | 246 | 147 | 417.3731 | 3.86615 | 417.3573 | 417.4005 | 3.813356 | 3.930708 |
| 856_12 | Peak_298 | 226 | 147 | 387.3617 | 3.866801 | 387.3445 | 387.3834 | 3.813356 | 3.930708 |
| 856_12 | Peak_412 | 321 | 147 | 855.7434 | 3.866589 | 855.6947 | 855.8103 | 3.813356 | 3.930708 |
| 856_12 | Peak_001 | 67 | 147 | 91.05527 | 3.866422 | 91.04744 | 91.06361 | 3.813356 | 3.930708 |
| 856_12 | Peak_399 | 312 | 147 | 617.5129 | 3.866683 | 617.4752 | 617.5559 | 3.827645 | 3.930708 |
| 856_12 | Peak_288 | 218 | 147 | 373.3464 | 3.866296 | 373.3333 | 373.366 | 3.813356 | 3.930708 |
| 856_12 | Peak_398 | 311 | 147 | 616.5013 | 3.867182 | 616.4655 | 616.5321 | 3.813356 | 3.930708 |
| 856_12 | Peak_116 | 32 | 147 | 229.1951 | 3.867264 | 229.1818 | 229.2096 | 3.827645 | 3.930708 |
| 856_12 | Peak_087 | 97 | 147 | 191.1795 | 3.867209 | 191.1687 | 191.1922 | 3.827645 | 3.930708 |
| 856_12 | Peak_056 | 84 | 147 | 151.148 | 3.86624 | 151.1394 | 151.1585 | 3.813356 | 3.930708 |
| 856_12 | Peak_246 | 54 | 147 | 339.2891 | 3.866146 | 339.2758 | 339.3148 | 3.813356 | 3.930708 |
| 856_12 | Peak_120 | 33 | 147 | 233.2263 | 3.866457 | 233.2146 | 233.2405 | 3.813356 | 3.930708 |
| 856_12 | Peak_278 | 212 | 147 | 359.3308 | 3.865665 | 359.3179 | 359.3527 | 3.827645 | 3.930708 |
| 856_12 | Peak_023 | 72 | 147 | 111.1171 | 3.866833 | 111.1089 | 111.1268 | 3.813356 | 3.930708 |
| 856_12 | Peak_227 | 182 | 147 | 318.2504 | 3.866538 | 318.2331 | 318.2633 | 3.813356 | 3.930708 |
| 856_12 | Peak_391 | 305 | 147 | 582.4945 | 3.866287 | 582.4619 | 582.5266 | 3.813356 | 3.916297 |
| 856_12 | Peak_136 | 36 | 147 | 245.226 | 3.866531 | 245.2145 | 245.2388 | 3.813356 | 3.930708 |
| 856_12 | Peak_384 | 298 | 147 | 574.4894 | 3.865421 | 574.4568 | 574.5244 | 3.813356 | 3.930708 |
| 856_12 | Peak_092 | 26 | 147 | 199.1476 | 3.867287 | 199.1347 | 199.1606 | 3.827645 | 3.930708 |
| 856_12 | Peak_139 | 37 | 147 | 247.2418 | 3.865581 | 247.2301 | 247.2567 | 3.813356 | 3.930708 |
| 856_12 | Peak_260 | 56 | 147 | 345.2784 | 3.865654 | 345.2611 | 345.2925 | 3.813356 | 3.930708 |
| 856_12 | Peak_306 | 231 | 147 | 399.3618 | 3.866866 | 399.3455 | 399.3879 | 3.813356 | 3.916297 |
| 856_12 | Peak_385 | 299 | 147 | 575.5024 | 3.866155 | 575.4654 | 575.5365 | 3.813356 | 3.930708 |
| 856_12 | Peak_363 | 278 | 147 | 507.4543 | 3.866012 | 507.4246 | 507.485 | 3.813356 | 3.930708 |
| 856_12 | Peak_005 | 3 | 147 | 96.08971 | 3.867008 | 96.08245 | 96.09767 | 3.813356 | 3.930708 |
| 856_12 | Peak_324 | 247 | 147 | 418.3759 | 3.866521 | 418.3614 | 418.3989 | 3.813356 | 3.930708 |
| 856_12 | Peak_081 | 95 | 147 | 185.1323 | 3.86688 | 185.1204 | 185.1454 | 3.813356 | 3.930708 |
| 856_12 | Peak_107 | 106 | 147 | 217.195 | 3.866803 | 217.1842 | 217.2071 | 3.827645 | 3.930708 |
| 856_12 | Peak_155 | 38 | 147 | 263.2367 | 3.866149 | 263.2254 | 263.2529 | 3.813356 | 3.930708 |
| 856_12 | Peak_309 | 234 | 147 | 401.3772 | 3.866806 | 401.3621 | 401.3989 | 3.827645 | 3.930708 |
| 856_12 | Peak_237 | 52 | 147 | 331.2624 | 3.867416 | 331.2458 | 331.2766 | 3.827645 | 3.930708 |
| 856_12 | Peak_103 | 29 | 147 | 213.1626 | 3.866869 | 213.1426 | 213.1756 | 3.827645 | 3.916297 |
| 856_12 | Peak_387 | 301 | 147 | 576.5057 | 3.864695 | 576.4681 | 576.5427 | 3.813356 | 3.930708 |
| 856_12 | Peak_109 | 31 | 147 | 219.2102 | 3.866207 | 219.1983 | 219.2234 | 3.827645 | 3.930708 |
| 856_12 | Peak_313 | 238 | 147 | 403.3557 | 3.866134 | 403.3384 | 403.3781 | 3.813356 | 3.916297 |
| 856_12 | Peak_134 | 121 | 147 | 244.2138 | 3.866066 | 244.201 | 244.2275 | 3.813356 | 3.916297 |
| 856_12 | Peak_067 | 20 | 147 | 171.1168 | 3.867148 | 171.1056 | 171.1278 | 3.827645 | 3.930708 |
| 856_12 | Peak_226 | 181 | 147 | 317.2843 | 3.866736 | 317.2744 | 317.3021 | 3.827645 | 3.916297 |
| 856_12 | Peak_361 | 276 | 147 | 501.3922 | 3.865976 | 501.3665 | 501.4108 | 3.827645 | 3.916297 |
| 856_12 | Peak_229 | 50 | 147 | 319.263 | 3.866155 | 319.2461 | 319.284 | 3.813356 | 3.916297 |
| 856_12 | Peak_316 | 240 | 147 | 413.3769 | 3.867476 | 413.3615 | 413.4016 | 3.827645 | 3.930708 |
| 856_12 | Peak_383 | 297 | 147 | 573.4869 | 3.865719 | 573.4524 | 573.52 | 3.827645 | 3.930708 |
| 856_12 | Peak_397 | 310 | 147 | 615.4976 | 3.867239 | 615.4635 | 615.5266 | 3.827645 | 3.930708 |
| 856_12 | Peak_198 | 43 | 147 | 290.2559 | 3.866462 | 290.242 | 290.2708 | 3.827645 | 3.916297 |
| 856_12 | Peak_421 | 327 | 147 | 857.7436 | 3.868366 | 857.7067 | 857.7811 | 3.827645 | 3.930708 |
| 856_12 | Peak_340 | 62 | 147 | 445.3664 | 3.866386 | 445.3506 | 445.3804 | 3.827645 | 3.916297 |
| 856_12 | Peak_021 | 7 | 147 | 110.1052 | 3.86584 | 110.098 | 110.1143 | 3.813356 | 3.930708 |
| 856_12 | Peak_300 | 227 | 147 | 388.3653 | 3.865725 | 388.348 | 388.387 | 3.813356 | 3.930708 |
| 856_12 | Peak_100 | 103 | 147 | 205.1949 | 3.866351 | 205.1835 | 205.2077 | 3.827645 | 3.930708 |
| 856_12 | Peak_277 | 211 | 147 | 359.2941 | 3.8674 | 359.2724 | 359.3072 | 3.827645 | 3.930708 |
| 856_12 | Peak_212 | 47 | 147 | 305.247 | 3.866787 | 305.232 | 305.2616 | 3.827645 | 3.930708 |
| 856_12 | Peak_145 | 128 | 147 | 257.2252 | 3.867245 | 257.2142 | 257.2346 | 3.813356 | 3.930708 |
| 856_12 | Peak_390 | 304 | 147 | 581.4918 | 3.86632 | 581.4608 | 581.5255 | 3.813356 | 3.930708 |
| 856_12 | Peak_348 | 264 | 147 | 459.3824 | 3.86667 | 459.3672 | 459.4005 | 3.827645 | 3.916297 |
| 856_12 | Peak_066 | 88 | 147 | 165.1637 | 3.865846 | 165.1539 | 165.1738 | 3.827645 | 3.916297 |
| 856_12 | Peak_333 | 256 | 147 | 431.3875 | 3.867076 | 431.3752 | 431.4104 | 3.827645 | 3.930708 |
| 856_12 | Peak_118 | 113 | 147 | 231.2106 | 3.867186 | 231.1992 | 231.2228 | 3.827645 | 3.930708 |
| 856_12 | Peak_151 | 134 | 147 | 261.2579 | 3.867311 | 261.2505 | 261.271 | 3.827645 | 3.916297 |
| 856_12 | Peak_367 | 281 | 147 | 516.4121 | 3.866831 | 516.3859 | 516.4468 | 3.827645 | 3.916297 |
| 856_12 | Peak_290 | 220 | 147 | 374.3493 | 3.866196 | 374.3349 | 374.3704 | 3.827645 | 3.930708 |
| 856_12 | Peak_307 | 232 | 147 | 400.3657 | 3.866198 | 400.3504 | 400.3871 | 3.827645 | 3.930708 |
| 856_12 | Peak_330 | 253 | 147 | 429.3725 | 3.866335 | 429.3489 | 429.3957 | 3.827645 | 3.916297 |
| 856_12 | Peak_199 | 44 | 147 | 291.2316 | 3.866946 | 291.2167 | 291.2456 | 3.827645 | 3.916297 |
| 856_12 | Peak_366 | 280 | 147 | 515.4081 | 3.866894 | 515.3824 | 515.4369 | 3.827645 | 3.916297 |
| 856_12 | Peak_326 | 249 | 147 | 427.356 | 3.867256 | 427.3389 | 427.371 | 3.827645 | 3.916297 |
| 856_12 | Peak_362 | 277 | 147 | 502.3965 | 3.867276 | 502.3721 | 502.4195 | 3.827645 | 3.916297 |
| 856_12 | Peak_338 | 261 | 147 | 443.388 | 3.866941 | 443.3689 | 443.4135 | 3.827645 | 3.916297 |
| 856_12 | Peak_112 | 109 | 147 | 221.2263 | 3.866284 | 221.2155 | 221.2386 | 3.813356 | 3.916297 |
| 856_12 | Peak_287 | 217 | 147 | 373.3107 | 3.865738 | 373.2924 | 373.3251 | 3.827645 | 3.916297 |
| 856_12 | Peak_147 | 130 | 147 | 259.2057 | 3.867722 | 259.1943 | 259.2171 | 3.827645 | 3.916297 |
| 856_12 | Peak_351 | 267 | 147 | 473.3981 | 3.866151 | 473.383 | 473.426 | 3.827645 | 3.930708 |
| 856_12 | Peak_240 | 53 | 147 | 333.2788 | 3.867586 | 333.2629 | 333.2964 | 3.827645 | 3.930708 |
| 856_12 | Peak_194 | 163 | 147 | 287.2363 | 3.872635 | 287.2223 | 287.2486 | 3.827645 | 3.930708 |
| 856_12 | Peak_130 | 35 | 147 | 241.1944 | 3.867256 | 241.1818 | 241.2081 | 3.813356 | 3.916297 |
| 856_12 | Peak_223 | 49 | 147 | 315.2682 | 3.8671 | 315.2537 | 315.2837 | 3.813356 | 3.916297 |
| 856_12 | Peak_138 | 123 | 147 | 247.2064 | 3.867826 | 247.1923 | 247.2167 | 3.827645 | 3.930708 |
| 856_12 | Peak_211 | 172 | 147 | 304.2719 | 3.866349 | 304.2586 | 304.2882 | 3.813356 | 3.916297 |
| 856_12 | Peak_381 | 295 | 147 | 560.4732 | 3.866227 | 560.4424 | 560.5059 | 3.813356 | 3.916297 |
| 856_12 | Peak_115 | 112 | 147 | 227.1792 | 3.867409 | 227.1669 | 227.1924 | 3.827645 | 3.916297 |
| 856_12 | Peak_179 | 152 | 147 | 277.2155 | 3.866702 | 277.2041 | 277.2276 | 3.813356 | 3.916297 |
| 856_12 | Peak_311 | 236 | 147 | 402.3801 | 3.866744 | 402.3666 | 402.3978 | 3.813356 | 3.930708 |
| 856_12 | Peak_356 | 271 | 147 | 487.3737 | 3.866399 | 487.3569 | 487.3849 | 3.827645 | 3.916297 |
| 856_12 | Peak_205 | 45 | 147 | 301.2524 | 3.867228 | 301.2402 | 301.2647 | 3.827645 | 3.916297 |
| 856_12 | Peak_315 | 61 | 147 | 413.3397 | 3.867316 | 413.3242 | 413.35 | 3.827645 | 3.916297 |
| 856_12 | Peak_026 | 74 | 147 | 117.0704 | 3.866959 | 117.0622 | 117.079 | 3.827645 | 3.930708 |
| 856_12 | Peak_261 | 200 | 147 | 345.3157 | 3.867178 | 345.3057 | 345.3319 | 3.827645 | 3.930708 |
| 856_12 | Peak_178 | 40 | 147 | 276.2403 | 3.867491 | 276.2273 | 276.2554 | 3.827645 | 3.916297 |
| 856_12 | Peak_332 | 255 | 147 | 431.3512 | 3.86805 | 431.3371 | 431.3664 | 3.827645 | 3.916297 |
| 856_12 | Peak_342 | 62 | 147 | 446.368 | 3.865633 | 446.349 | 446.3848 | 3.827645 | 3.916297 |
| 856_12 | Peak_083 | 24 | 147 | 188.1523 | 3.866342 | 188.1421 | 188.1634 | 3.813356 | 3.916297 |
| 856_12 | Peak_319 | 242 | 147 | 415.3554 | 3.867947 | 415.3354 | 415.3699 | 3.827645 | 3.916297 |
| 856_12 | Peak_380 | 294 | 147 | 559.4699 | 3.867317 | 559.4437 | 559.5004 | 3.827645 | 3.916297 |
| 856_12 | Peak_208 | 46 | 147 | 303.2324 | 3.866534 | 303.2181 | 303.2426 | 3.827645 | 3.916297 |
| 856_12 | Peak_336 | 259 | 147 | 441.3717 | 3.867409 | 441.3501 | 441.3917 | 3.827645 | 3.930708 |
| 856_12 | Peak_281 | 214 | 147 | 360.3341 | 3.866121 | 360.322 | 360.3541 | 3.827645 | 3.916297 |
| 856_12 | Peak_295 | 224 | 147 | 385.3454 | 3.866767 | 385.3303 | 385.3663 | 3.827645 | 3.916297 |
| 856_12 | Peak_314 | 239 | 147 | 404.36 | 3.865199 | 404.3425 | 404.3794 | 3.827645 | 3.916297 |
| 856_12 | Peak_070 | 21 | 147 | 174.1363 | 3.866066 | 174.1266 | 174.1471 | 3.827645 | 3.916297 |
| 856_12 | Peak_318 | 241 | 147 | 414.3802 | 3.866727 | 414.3665 | 414.401 | 3.827645 | 3.930708 |
| 856_12 | Peak_148 | 131 | 147 | 259.2418 | 3.867106 | 259.2307 | 259.2557 | 3.827645 | 3.930708 |
| 856_12 | Peak_339 | 262 | 147 | 444.3917 | 3.866202 | 444.3741 | 444.4157 | 3.827645 | 3.916297 |
| 856_12 | Peak_238 | 188 | 147 | 331.2983 | 3.867192 | 331.2869 | 331.3126 | 3.827645 | 3.916297 |
| 856_12 | Peak_175 | 150 | 147 | 275.2008 | 3.866781 | 275.189 | 275.2101 | 3.827645 | 3.916297 |
| 856_12 | Peak_352 | 268 | 147 | 474.3996 | 3.86669 | 474.3816 | 474.4216 | 3.827645 | 3.930708 |
| 856_12 | Peak_405 | 315 | 147 | 730.6363 | 3.865976 | 730.5986 | 730.6787 | 3.827645 | 3.916297 |
| 856_12 | Peak_204 | 169 | 147 | 299.237 | 3.867287 | 299.2249 | 299.2493 | 3.827645 | 3.916297 |
| 856_12 | Peak_280 | 213 | 147 | 360.2975 | 3.867746 | 360.2818 | 360.3086 | 3.827645 | 3.916297 |
| 856_12 | Peak_172 | 147 | 147 | 271.2418 | 3.86659 | 271.2297 | 271.2576 | 3.813356 | 3.916297 |
| 856_12 | Peak_245 | 192 | 147 | 339.2682 | 3.866483 | 339.2524 | 339.2758 | 3.827645 | 3.916297 |
| 856_12 | Peak_119 | 114 | 147 | 233.1907 | 3.86496 | 233.178 | 233.2017 | 3.827645 | 3.916297 |
| 856_12 | Peak_234 | 187 | 147 | 329.2476 | 3.867841 | 329.2347 | 329.2578 | 3.827645 | 3.916297 |
| 856_12 | Peak_349 | 265 | 147 | 460.3862 | 3.867455 | 460.3692 | 460.4116 | 3.827645 | 3.930708 |
| 856_12 | Peak_263 | 201 | 147 | 347.2948 | 3.867362 | 347.2836 | 347.3099 | 3.827645 | 3.916297 |
| 856_12 | Peak_043 | 13 | 147 | 136.1204 | 3.86729 | 136.111 | 136.1291 | 3.827645 | 3.916297 |
| 856_12 | Peak_129 | 34 | 147 | 240.2408 | 3.867626 | 240.227 | 240.2555 | 3.813356 | 3.930708 |
| 856_12 | Peak_174 | 149 | 147 | 273.2574 | 3.866666 | 273.2466 | 273.2699 | 3.827645 | 3.916297 |
| 856_12 | Peak_072 | 22 | 147 | 176.1515 | 3.867366 | 176.1422 | 176.1609 | 3.827645 | 3.916297 |
| 856_12 | Peak_302 | 60 | 147 | 389.3401 | 3.868155 | 389.325 | 389.3612 | 3.827645 | 3.916297 |
| 856_12 | Peak_337 | 260 | 147 | 442.3745 | 3.866404 | 442.3589 | 442.3886 | 3.827645 | 3.916297 |
| 856_12 | Peak_262 | 56 | 147 | 346.2822 | 3.867009 | 346.2663 | 346.2952 | 3.827645 | 3.916297 |
| 856_12 | Peak_196 | 165 | 147 | 289.2169 | 3.86609 | 289.2041 | 289.2257 | 3.827645 | 3.916297 |
| 856_12 | Peak_160 | 138 | 147 | 265.2525 | 3.867249 | 265.2399 | 265.2652 | 3.827645 | 3.916297 |
| 856_12 | Peak_035 | 76 | 147 | 125.0966 | 3.86689 | 125.0895 | 125.1052 | 3.827645 | 3.916297 |
| 856_12 | Peak_009 | 69 | 147 | 99.08123 | 3.86852 | 99.07461 | 99.08866 | 3.827645 | 3.916297 |
| 856_12 | Peak_327 | 250 | 147 | 427.391 | 3.867195 | 427.3798 | 427.409 | 3.827645 | 3.916297 |
| 856_12 | Peak_144 | 127 | 147 | 255.2104 | 3.865879 | 255.1966 | 255.2237 | 3.813356 | 3.916297 |
| 856_12 | Peak_058 | 86 | 147 | 157.1009 | 3.864426 | 157.0935 | 157.1095 | 3.827645 | 3.916297 |
| 856_12 | Peak_382 | 296 | 147 | 564.4942 | 3.866484 | 564.4629 | 564.5232 | 3.827645 | 3.916297 |
| 856_12 | Peak_331 | 254 | 147 | 430.3752 | 3.866081 | 430.3556 | 430.3966 | 3.827645 | 3.916297 |
| 856_12 | Peak_358 | 273 | 147 | 488.3797 | 3.867982 | 488.3577 | 488.3951 | 3.827645 | 3.916297 |
| 856_12 | Peak_095 | 27 | 147 | 202.1666 | 3.86739 | 202.1558 | 202.1779 | 3.827645 | 3.930708 |
| 856_12 | Peak_297 | 59 | 147 | 387.3233 | 3.868094 | 387.3084 | 387.3334 | 3.827645 | 3.930708 |
| 856_12 | Peak_030 | 9 | 147 | 122.1043 | 3.865764 | 122.0954 | 122.1141 | 3.827645 | 3.916297 |
| 856_12 | Peak_335 | 258 | 147 | 432.3909 | 3.865656 | 432.3784 | 432.4078 | 3.827645 | 3.916297 |
| 856_12 | Peak_251 | 194 | 147 | 341.2838 | 3.868007 | 341.2677 | 341.2964 | 3.827645 | 3.916297 |
| 856_12 | Peak_239 | 52 | 147 | 332.2666 | 3.867591 | 332.2536 | 332.2819 | 3.827645 | 3.916297 |
| 856_12 | Peak_149 | 132 | 147 | 261.1839 | 3.865897 | 261.1729 | 261.1911 | 3.827645 | 3.930708 |
| 856_12 | Peak_255 | 55 | 147 | 343.263 | 3.86904 | 343.2471 | 343.2758 | 3.827645 | 3.930708 |
| 856_12 | Peak_305 | 230 | 147 | 399.3229 | 3.866502 | 399.3089 | 399.3314 | 3.827645 | 3.916297 |
| 856_12 | Peak_171 | 146 | 147 | 271.206 | 3.868161 | 271.1948 | 271.2158 | 3.827645 | 3.916297 |
| 856_12 | Peak_346 | 64 | 147 | 455.3866 | 3.867608 | 455.3644 | 455.4036 | 3.827645 | 3.916297 |
| 856_12 | Peak_283 | 216 | 147 | 361.3085 | 3.866999 | 361.2952 | 361.3221 | 3.827645 | 3.916297 |
| 856_12 | Peak_406 | 316 | 147 | 744.6493 | 3.867231 | 744.6216 | 744.6871 | 3.827645 | 3.930708 |
| 856_12 | Peak_085 | 25 | 147 | 190.1673 | 3.866685 | 190.1572 | 190.1767 | 3.827645 | 3.916297 |
| 856_12 | Peak_328 | 251 | 147 | 428.3587 | 3.867449 | 428.3404 | 428.3725 | 3.827645 | 3.916297 |
| 856_12 | Peak_228 | 183 | 147 | 318.2873 | 3.867428 | 318.2784 | 318.2985 | 3.827645 | 3.916297 |
| 856_12 | Peak_154 | 136 | 147 | 263.2 | 3.865417 | 263.1887 | 263.2094 | 3.827645 | 3.916297 |
| 856_12 | Peak_334 | 257 | 147 | 432.3532 | 3.867729 | 432.3373 | 432.3667 | 3.827645 | 3.916297 |
| 856_12 | Peak_308 | 233 | 147 | 401.3405 | 3.866495 | 401.3253 | 401.3508 | 3.827645 | 3.916297 |
| 856_12 | Peak_108 | 107 | 147 | 219.1747 | 3.867458 | 219.1628 | 219.1858 | 3.827645 | 3.916297 |
| 856_12 | Peak_375 | 289 | 147 | 542.4651 | 3.865009 | 542.441 | 542.487 | 3.80249 | 3.916297 |
| 856_12 | Peak_247 | 54 | 147 | 340.2926 | 3.866897 | 340.2801 | 340.3114 | 3.827645 | 3.916297 |
| 856_12 | Peak_273 | 208 | 147 | 357.2788 | 3.869322 | 357.2632 | 357.2926 | 3.827645 | 3.916297 |
| 856_12 | Peak_010 | 70 | 147 | 99.11738 | 3.866202 | 99.10974 | 99.12379 | 3.827645 | 3.916297 |
| 856_12 | Peak_202 | 167 | 147 | 293.2472 | 3.865643 | 293.234 | 293.2605 | 3.827645 | 3.916297 |
| 856_12 | Peak_192 | 42 | 147 | 285.2577 | 3.86737 | 285.2473 | 285.2712 | 3.827645 | 3.916297 |
| 856_12 | Peak_099 | 102 | 147 | 205.1584 | 3.866175 | 205.1491 | 205.1693 | 3.827645 | 3.916297 |
| 856_12 | Peak_065 | 19 | 147 | 164.1512 | 3.868708 | 164.1432 | 164.1595 | 3.813356 | 3.930708 |
| 856_12 | Peak_091 | 100 | 147 | 197.1323 | 3.866077 | 197.122 | 197.1418 | 3.827645 | 3.916297 |
| 856_12 | Peak_235 | 51 | 147 | 329.2832 | 3.866662 | 329.2706 | 329.3013 | 3.827645 | 3.916297 |
| 856_12 | Peak_289 | 219 | 147 | 374.3121 | 3.867745 | 374.3021 | 374.3212 | 3.827645 | 3.916297 |
| 856_12 | Peak_123 | 116 | 147 | 235.2421 | 3.868644 | 235.2323 | 235.2518 | 3.827645 | 3.916297 |
| 856_12 | Peak_222 | 179 | 147 | 315.2317 | 3.868331 | 315.2186 | 315.2436 | 3.827645 | 3.916297 |
| 856_12 | Peak_173 | 148 | 147 | 273.2214 | 3.867032 | 273.2093 | 273.2326 | 3.827645 | 3.916297 |
| 856_12 | Peak_195 | 164 | 147 | 287.2735 | 3.867404 | 287.263 | 287.2869 | 3.827645 | 3.916297 |
| 856_12 | Peak_372 | 286 | 147 | 529.4238 | 3.866771 | 529.3998 | 529.4453 | 3.827645 | 3.916297 |
| 856_12 | Peak_146 | 129 | 147 | 257.2458 | 3.867566 | 257.2391 | 257.2595 | 3.827645 | 3.916297 |
| 856_12 | Peak_167 | 143 | 147 | 269.2264 | 3.873213 | 269.2133 | 269.2388 | 3.827645 | 3.930708 |
| 856_12 | Peak_191 | 162 | 147 | 285.2216 | 3.865035 | 285.2092 | 285.233 | 3.827645 | 3.916297 |
| 856_12 | Peak_317 | 61 | 147 | 414.3441 | 3.867864 | 414.3263 | 414.355 | 3.827645 | 3.916297 |
| 856_12 | Peak_048 | 14 | 147 | 146.1051 | 3.866039 | 146.0977 | 146.113 | 3.827645 | 3.916297 |
| 856_12 | Peak_135 | 122 | 147 | 245.1906 | 3.866354 | 245.1813 | 245.199 | 3.827645 | 3.916297 |
| 856_12 | Peak_369 | 283 | 147 | 524.4532 | 3.8646 | 524.4327 | 524.4715 | 3.813356 | 3.916297 |
| 856_12 | Peak_233 | 186 | 147 | 327.2669 | 3.867691 | 327.2528 | 327.2809 | 3.827645 | 3.916297 |
| 856_12 | Peak_060 | 17 | 147 | 160.1209 | 3.866819 | 160.1137 | 160.128 | 3.827645 | 3.916297 |
| 856_12 | Peak_177 | 151 | 147 | 275.2734 | 3.868546 | 275.2663 | 275.285 | 3.827645 | 3.916297 |
| 856_12 | Peak_321 | 244 | 147 | 416.3597 | 3.864447 | 416.3457 | 416.3716 | 3.827645 | 3.916297 |
| 856_12 | Peak_213 | 47 | 147 | 306.2507 | 3.865575 | 306.239 | 306.2637 | 3.827645 | 3.916297 |
| 856_12 | Peak_114 | 111 | 147 | 225.1628 | 3.867021 | 225.1524 | 225.1715 | 3.827645 | 3.916297 |
| 856_12 | Peak_143 | 126 | 147 | 249.2573 | 3.865608 | 249.2472 | 249.2672 | 3.827645 | 3.916297 |
| 856_12 | Peak_285 | 58 | 147 | 371.3304 | 3.867802 | 371.3205 | 371.345 | 3.827645 | 3.916297 |
| 856_12 | Peak_230 | 50 | 147 | 320.266 | 3.866446 | 320.2507 | 320.2785 | 3.827645 | 3.916297 |
| 856_12 | Peak_046 | 80 | 147 | 139.1116 | 3.867705 | 139.103 | 139.1196 | 3.827645 | 3.916297 |
| 856_12 | Peak_106 | 30 | 147 | 216.1827 | 3.865848 | 216.1723 | 216.1931 | 3.827645 | 3.916297 |
| 856_12 | Peak_292 | 222 | 147 | 375.3246 | 3.866222 | 375.3105 | 375.3406 | 3.827645 | 3.916297 |
| 856_12 | Peak_006 | 68 | 147 | 97.06601 | 3.865791 | 97.05898 | 97.07427 | 3.827645 | 3.930708 |
| 856_12 | Peak_357 | 272 | 147 | 487.4146 | 3.867962 | 487.4036 | 487.4348 | 3.827645 | 3.916297 |
| 856_12 | Peak_410 | 319 | 147 | 837.736 | 3.866746 | 837.7131 | 837.7662 | 3.827645 | 3.916297 |
| 856_12 | Peak_054 | 16 | 147 | 150.1358 | 3.865824 | 150.1277 | 150.145 | 3.827645 | 3.916297 |
| 856_12 | Peak_344 | 63 | 147 | 451.3777 | 3.868205 | 451.3491 | 451.4031 | 3.827645 | 3.916297 |
| 856_12 | Peak_102 | 105 | 147 | 211.1477 | 3.867819 | 211.1381 | 211.1586 | 3.827645 | 3.916297 |
| 856_12 | Peak_180 | 153 | 147 | 277.2521 | 3.868014 | 277.244 | 277.2628 | 3.827645 | 3.916297 |
| 856_12 | Peak_181 | 154 | 147 | 279.2314 | 3.866139 | 279.2194 | 279.243 | 3.827645 | 3.930708 |
| 856_12 | Peak_039 | 11 | 147 | 132.0895 | 3.866026 | 132.0819 | 132.0981 | 3.827645 | 3.916297 |
| 856_12 | Peak_215 | 174 | 147 | 311.2368 | 3.866248 | 311.2266 | 311.2465 | 3.827645 | 3.916297 |
| 856_12 | Peak_214 | 173 | 147 | 307.2618 | 3.865625 | 307.2476 | 307.2748 | 3.827645 | 3.916297 |
| 856_12 | Peak_022 | 71 | 147 | 111.0813 | 3.867217 | 111.0732 | 111.0896 | 3.827645 | 3.916297 |
| 856_12 | Peak_033 | 10 | 147 | 124.12 | 3.86602 | 124.111 | 124.1283 | 3.827645 | 3.916297 |
| 856_12 | Peak_073 | 89 | 147 | 177.1284 | 3.864885 | 177.1196 | 177.1365 | 3.827645 | 3.916297 |
| 856_12 | Peak_232 | 185 | 147 | 325.2516 | 3.865987 | 325.2361 | 325.2641 | 3.827645 | 3.916297 |
| 856_12 | Peak_036 | 77 | 147 | 125.1325 | 3.865892 | 125.1242 | 125.1416 | 3.827645 | 3.916297 |
| 856_12 | Peak_396 | 309 | 147 | 602.519 | 3.863317 | 602.4988 | 602.5473 | 3.827645 | 3.916297 |
| 856_12 | Peak_373 | 287 | 147 | 530.4262 | 3.867496 | 530.4071 | 530.4461 | 3.827645 | 3.916297 |
| 856_12 | Peak_140 | 37 | 147 | 248.2458 | 3.867014 | 248.2365 | 248.2565 | 3.827645 | 3.916297 |
| 856_12 | Peak_274 | 209 | 147 | 357.3148 | 3.866909 | 357.3033 | 357.3299 | 3.827645 | 3.916297 |
| 856_12 | Peak_077 | 91 | 147 | 183.116 | 3.86647 | 183.1075 | 183.1247 | 3.827645 | 3.916297 |
| 856_12 | Peak_347 | 64 | 147 | 456.3912 | 3.865873 | 456.374 | 456.4072 | 3.827645 | 3.916297 |
| 856_12 | Peak_206 | 170 | 147 | 301.2882 | 3.865363 | 301.2794 | 301.3015 | 3.827645 | 3.916297 |
| 856_12 | Peak_117 | 32 | 147 | 230.1986 | 3.870186 | 230.1873 | 230.2087 | 3.827645 | 3.930708 |
| 856_12 | Peak_157 | 38 | 147 | 264.2407 | 3.86539 | 264.2294 | 264.2524 | 3.827645 | 3.916297 |
| 856_12 | Peak_301 | 228 | 147 | 389.3047 | 3.867294 | 389.2916 | 389.3166 | 3.827645 | 3.930708 |
| 856_12 | Peak_076 | 90 | 147 | 179.1794 | 3.866 | 179.1712 | 179.1882 | 3.827645 | 3.916297 |
| 856_12 | Peak_378 | 292 | 147 | 545.4817 | 3.869808 | 545.4697 | 545.4994 | 3.842423 | 3.930708 |
| 856_12 | Peak_188 | 159 | 147 | 283.2419 | 3.866955 | 283.2317 | 283.2554 | 3.827645 | 3.916297 |
| 856_12 | Peak_325 | 248 | 147 | 423.3609 | 3.868989 | 423.3362 | 423.3798 | 3.827645 | 3.916297 |
| 856_12 | Peak_296 | 225 | 147 | 386.3496 | 3.866036 | 386.3339 | 386.3672 | 3.827645 | 3.916297 |
| 856_12 | Peak_374 | 288 | 147 | 541.4617 | 3.865651 | 541.4387 | 541.4847 | 3.827645 | 3.916297 |
| 856_12 | Peak_051 | 15 | 147 | 148.1204 | 3.867037 | 148.1127 | 148.1281 | 3.827645 | 3.916297 |
| 856_12 | Peak_044 | 78 | 147 | 137.0973 | 3.86688 | 137.0876 | 137.1075 | 3.827645 | 3.916297 |
| 856_12 | Peak_256 | 197 | 147 | 343.2997 | 3.866138 | 343.2889 | 343.3125 | 3.827645 | 3.916297 |
| 856_12 | Peak_282 | 215 | 147 | 361.2737 | 3.865644 | 361.2577 | 361.2872 | 3.827645 | 3.916297 |
| 856_12 | Peak_127 | 118 | 147 | 239.1808 | 3.867955 | 239.1693 | 239.1933 | 3.827645 | 3.916297 |
| 856_12 | Peak_141 | 124 | 147 | 249.1838 | 3.867909 | 249.1736 | 249.1937 | 3.827645 | 3.930708 |
| 856_12 | Peak_310 | 235 | 147 | 402.3421 | 3.865614 | 402.3298 | 402.3525 | 3.827645 | 3.916297 |
| 856_12 | Peak_203 | 168 | 147 | 297.2652 | 3.867444 | 297.2479 | 297.2893 | 3.827645 | 3.916297 |
| 856_12 | Peak_137 | 36 | 147 | 246.23 | 3.866137 | 246.219 | 246.2412 | 3.827645 | 3.916297 |
| 856_12 | Peak_368 | 282 | 147 | 523.4495 | 3.866338 | 523.4279 | 523.4731 | 3.827645 | 3.916297 |
| 856_12 | Peak_304 | 229 | 147 | 395.3319 | 3.866602 | 395.322 | 395.3445 | 3.827645 | 3.916297 |
| 856_12 | Peak_224 | 49 | 147 | 316.2717 | 3.867498 | 316.2569 | 316.2846 | 3.827645 | 3.930708 |
| 856_12 | Peak_359 | 274 | 147 | 488.4192 | 3.867321 | 488.4107 | 488.4356 | 3.827645 | 3.916297 |
| 856_12 | Peak_041 | 12 | 147 | 134.1047 | 3.864744 | 134.0962 | 134.1126 | 3.827645 | 3.916297 |
| 856_12 | Peak_341 | 263 | 147 | 445.4012 | 3.867236 | 445.3923 | 445.4161 | 3.842423 | 3.930708 |
| 856_12 | Peak_303 | 60 | 147 | 390.3451 | 3.865865 | 390.3339 | 390.3618 | 3.827645 | 3.916297 |
| 856_12 | Peak_075 | 23 | 147 | 178.1668 | 3.867723 | 178.1581 | 178.175 | 3.827645 | 3.916297 |
| 856_12 | Peak_329 | 252 | 147 | 428.3959 | 3.86779 | 428.3842 | 428.4134 | 3.827645 | 3.916297 |
| 856_12 | Peak_024 | 73 | 147 | 113.0963 | 3.867098 | 113.0891 | 113.1041 | 3.827645 | 3.916297 |
| 856_12 | Peak_028 | 8 | 147 | 120.0891 | 3.866639 | 120.0809 | 120.0979 | 3.813356 | 3.930708 |
| 856_12 | Peak_216 | 175 | 147 | 311.2636 | 3.866519 | 311.2515 | 311.2838 | 3.827645 | 3.916297 |
| 856_12 | Peak_121 | 33 | 147 | 234.2295 | 3.867284 | 234.2202 | 234.2397 | 3.827645 | 3.916297 |
| 856_12 | Peak_098 | 28 | 147 | 204.1831 | 3.867382 | 204.1738 | 204.1919 | 3.827645 | 3.916297 |
| 856_12 | Peak_379 | 293 | 147 | 550.49 | 3.86687 | 550.4723 | 550.512 | 3.827645 | 3.916297 |
| 856_12 | Peak_291 | 221 | 147 | 375.2875 | 3.867144 | 375.2749 | 375.2995 | 3.827645 | 3.916297 |
| 856_12 | Peak_201 | 44 | 147 | 292.2356 | 3.864427 | 292.2245 | 292.2462 | 3.827645 | 3.916297 |
| 856_12 | Peak_299 | 59 | 147 | 388.3284 | 3.865592 | 388.3174 | 388.3369 | 3.827645 | 3.916297 |
| 856_12 | Peak_122 | 115 | 147 | 235.17 | 3.866556 | 235.1587 | 235.1804 | 3.827645 | 3.916297 |
| 856_12 | Peak_104 | 29 | 147 | 214.1664 | 3.865137 | 214.1577 | 214.1763 | 3.827645 | 3.916297 |
| 856_12 | Peak_241 | 53 | 147 | 334.2821 | 3.867818 | 334.2712 | 334.2945 | 3.827645 | 3.916297 |
| 856_12 | Peak_320 | 243 | 147 | 415.3908 | 3.866758 | 415.3814 | 415.4102 | 3.827645 | 3.916297 |
| 856_12 | Peak_093 | 26 | 147 | 200.1521 | 3.867374 | 200.1418 | 200.1618 | 3.827645 | 3.916297 |
| 856_12 | Peak_062 | 18 | 147 | 162.1357 | 3.86701 | 162.1276 | 162.1438 | 3.813356 | 3.916297 |
| 856_12 | Peak_210 | 46 | 147 | 304.2371 | 3.869246 | 304.2266 | 304.2463 | 3.827645 | 3.916297 |
| 856_12 | Peak_345 | 63 | 147 | 452.38 | 3.868809 | 452.3603 | 452.4023 | 3.827645 | 3.916297 |
| 856_12 | Peak_231 | 184 | 147 | 321.2776 | 3.868501 | 321.2644 | 321.2948 | 3.827645 | 3.916297 |
| 856_12 | Peak_312 | 237 | 147 | 403.2854 | 3.867014 | 403.2732 | 403.2959 | 3.827645 | 3.916297 |
| 856_12 | Peak_018 | 6 | 147 | 108.0897 | 3.865256 | 108.0812 | 108.0973 | 3.813356 | 3.916297 |
| 856_12 | Peak_236 | 51 | 147 | 330.2885 | 3.867404 | 330.278 | 330.301 | 3.827645 | 3.916297 |
| 856_12 | Peak_207 | 45 | 147 | 302.2565 | 3.868876 | 302.2455 | 302.2676 | 3.827645 | 3.916297 |
| 856_12 | Peak_031 | 75 | 147 | 123.0808 | 3.868352 | 123.0737 | 123.0894 | 3.827645 | 3.916297 |
| 856_12 | Peak_142 | 125 | 147 | 249.2226 | 3.86781 | 249.2115 | 249.2316 | 3.827645 | 3.916297 |
| 856_12 | Peak_159 | 137 | 147 | 265.2149 | 3.86614 | 265.2032 | 265.2261 | 3.827645 | 3.916297 |
| 856_12 | Peak_068 | 20 | 147 | 172.1205 | 3.868585 | 172.1116 | 172.1301 | 3.827645 | 3.916297 |
| 856_12 | Peak_350 | 266 | 147 | 466.3947 | 3.866395 | 466.3731 | 466.4128 | 3.827645 | 3.916297 |
| 856_12 | Peak_132 | 119 | 147 | 243.1738 | 3.8648 | 243.1654 | 243.1808 | 3.827645 | 3.916297 |
| 856_12 | Peak_322 | 245 | 147 | 417.2968 | 3.868732 | 417.2852 | 417.3054 | 3.827645 | 3.916297 |
| 856_12 | Peak_242 | 189 | 147 | 335.2603 | 3.865422 | 335.25 | 335.2707 | 3.827645 | 3.916297 |
| 856_12 | Peak_063 | 87 | 147 | 163.112 | 3.865412 | 163.105 | 163.1194 | 3.827645 | 3.916297 |
| 856_12 | Peak_294 | 223 | 147 | 385.3093 | 3.86443 | 385.2942 | 385.3192 | 3.827645 | 3.916297 |
| 856_12 | Peak_089 | 98 | 147 | 193.1944 | 3.868832 | 193.1862 | 193.2038 | 3.827645 | 3.916297 |
| 856_12 | Peak_086 | 96 | 147 | 191.1427 | 3.864369 | 191.1336 | 191.1512 | 3.827645 | 3.916297 |
| 856_12 | Peak_008 | 4 | 147 | 98.10561 | 3.8638 | 98.09915 | 98.11313 | 3.827645 | 3.916297 |
| 856_12 | Peak_267 | 203 | 147 | 353.2837 | 3.867919 | 353.2698 | 353.2937 | 3.827645 | 3.916297 |
| 856_12 | Peak_200 | 166 | 147 | 291.2682 | 3.866652 | 291.2601 | 291.2794 | 3.827645 | 3.916297 |
| 856_12 | Peak_096 | 101 | 147 | 203.1449 | 3.868529 | 203.1364 | 203.1525 | 3.827645 | 3.916297 |
| 856_12 | Peak_057 | 85 | 147 | 153.1267 | 3.867511 | 153.1189 | 153.1346 | 3.827645 | 3.916297 |
| 856_12 | Peak_243 | 190 | 147 | 337.2739 | 3.867085 | 337.2586 | 337.2897 | 3.827645 | 3.916297 |
| 856_12 | Peak_360 | 275 | 147 | 500.4536 | 3.862029 | 500.4409 | 500.4693 | 3.827645 | 3.916297 |
| 856_12 | Peak_113 | 110 | 147 | 223.1684 | 3.86885 | 223.1617 | 223.1785 | 3.827645 | 3.916297 |
| 856_12 | Peak_111 | 108 | 147 | 221.1913 | 3.866754 | 221.1819 | 221.2008 | 3.827645 | 3.916297 |
| 856_12 | Peak_110 | 31 | 147 | 220.2133 | 3.866714 | 220.2047 | 220.2215 | 3.827645 | 3.916297 |
| 856_12 | Peak_055 | 83 | 147 | 151.1131 | 3.866951 | 151.1064 | 151.1203 | 3.827645 | 3.916297 |
| 856_12 | Peak_131 | 35 | 147 | 242.1976 | 3.865396 | 242.1869 | 242.211 | 3.827645 | 3.916297 |
| 856_12 | Peak_052 | 82 | 147 | 149.0968 | 3.86825 | 149.09 | 149.1055 | 3.827645 | 3.916297 |
| 856_12 | Peak_090 | 99 | 147 | 195.1378 | 3.866805 | 195.1314 | 195.1452 | 3.827645 | 3.916297 |
| 856_12 | Peak_003 | 2 | 147 | 94.07431 | 3.864355 | 94.06752 | 94.08121 | 3.827645 | 3.916297 |
| 856_12 | Peak_016 | 5 | 147 | 106.0739 | 3.867697 | 106.067 | 106.0816 | 3.827645 | 3.916297 |
| 856_12 | Peak_408 | 317 | 147 | 772.6541 | 3.866772 | 772.6322 | 772.6792 | 3.827645 | 3.916297 |
| 856_12 | Peak_218 | 176 | 147 | 312.2747 | 3.865714 | 312.2608 | 312.2908 | 3.827645 | 3.916297 |
| 856_12 | Peak_286 | 58 | 147 | 372.3348 | 3.865821 | 372.3221 | 372.3466 | 3.827645 | 3.916297 |
| 856_12 | Peak_258 | 55 | 147 | 344.2669 | 3.867605 | 344.2546 | 344.2782 | 3.827645 | 3.916297 |
| 856_12 | Peak_193 | 42 | 147 | 286.2623 | 3.865712 | 286.2519 | 286.271 | 3.827645 | 3.916297 |

| | | | | | | | | MS/MS library |
|---|---|---|---|---|---|---|---|---|
| UnitMassFile_R | Name | Library. m.z | Library. RT | Slope | RSQ | UnitMass_File | R Cluster Number | annotation |
| 856_12 | Peak_34342 | | | 731.2411 | 0.774692 | 856 | 12 | |
| 856_12 | Peak_394 | | | 174.4919 | 0.866667 | 856 | 12 | |
| 856_12 | Peak_393 | | | 59.39108 | 0.594044 | 856 | 12 | |
| 856_12 | Peak_418 | | | 45.3658 | 0.891408 | 856 | 12 | |
| 856_12 | Peak_004 | | | 44.10979 | 0.881068 | 856 | 12 | |
| 856_12 | Peak_377 | | | 43.89625 | 0.869125 | 856 | 12 | |
| 856_12 | Peak_371 | | | 38.00673 | 0.776568 | 856 | 12 | |
| 856_12 | Peak_220 | | | 36.92729 | 0.843632 | 856 | 12 | |
| 856_12 | Peak_376 | | | 28.14988 | 0.71052 | 856 | 12 | |
| 856_12 | Peak_150 | | | 27.51612 | 0.788545 | 856 | 12 | |
| 856_12 | Peak_020 | | | 27.02113 | 0.868155 | 856 | 12 | |
| 856_12 | Peak_370 | | | 20.91009 | 0.749594 | 856 | 12 | |
| 856_12 | Peak_029 | | | 16.53867 | 0.868105 | 856 | 12 | |
| 856_12 | Peak_221 | | | 15.32847 | 0.845437 | 856 | 12 | |
| 856_12 | Peak_042 | | | 15.08577 | 0.784111 | 856 | 12 | |
| 856_12 | Peak_032 | | | 14.46325 | 0.877105 | 856 | 12 | |
| 856_12 | Peak_007 | | | 12.23836 | 0.866111 | 856 | 12 | |
| 856_12 | Peak_053 | | | 11.4509 | 0.853844 | 856 | 12 | |
| 856_12 | Peak_002 | | | 11.0205 | 0.835652 | 856 | 12 | |
| 856_12 | Peak_133 | | | 10.94425 | 0.769291 | 856 | 12 | |
| 856_12 | Peak_027 | | | 10.91345 | 0.839711 | 856 | 12 | |
| 856_12 | Peak_017 | | | 10.48487 | 0.787994 | 856 | 12 | |
| 856_12 | Peak_389 | | | 10.42732 | 0.860907 | 856 | 12 | |
| 856_12 | Peak_395 | | | 10.41854 | 0.610359 | 856 | 12 | |
| 856_12 | Peak_069 | | | 10.07363 | 0.827706 | 856 | 12 | |
| 856_12 | Peak_153 | | | 9.872565 | 0.78154 | 856 | 12 | |
| 856_12 | Peak_071 | | | 9.682427 | 0.866654 | 856 | 12 | |
| 856_12 | Peak_040 | | | 9.671766 | 0.825859 | 856 | 12 | |
| 856_12 | Peak_082 | | | 9.056999 | 0.816126 | 856 | 12 | |
| 856_12 | Peak_047 | | | 8.886721 | 0.862756 | 856 | 12 | |
| 856_12 | Peak_084 | | | 8.628944 | 0.83614 | 856 | 12 | |
| 856_12 | Peak_128 | | | 8.362815 | 0.838411 | 856 | 12 | |
| 856_12 | Peak_225 | | | 8.356146 | 0.789879 | 856 | 12 | |
| 856_12 | Peak_064 | | | 8.087782 | 0.779301 | 856 | 12 | |
| 856_12 | Peak_050 | | | 8.056002 | 0.84411 | 856 | 12 | |
| 856_12 | Peak_045 | | | 7.920539 | 0.835543 | 856 | 12 | |
| 856_12 | Peak_038 | | | 7.831259 | 0.851923 | 856 | 12 | |
| 856_12 | Peak_176 | | | 7.676622 | 0.82148 | 856 | 12 | |
| 856_12 | Peak_094 | | | 7.648837 | 0.841368 | 856 | 12 | |
| 856_12 | Peak_197 | | | 7.643458 | 0.820959 | 856 | 12 | |
| 856_12 | Peak_061 | | | 7.447835 | 0.830957 | 856 | 12 | |
| 856_12 | Peak_388 | | | 7.407234 | 0.868933 | 856 | 12 | |
| 856_12 | Peak_059 | | | 7.324365 | 0.874754 | 856 | 12 | |
| 856_12 | Peak_015 | | | 7.283251 | 0.830956 | 856 | 12 | |
| 856_12 | Peak_400 | | | 6.601511 | 0.858965 | 856 | 12 | |
| 856_12 | Peak_209 | | | 6.356712 | 0.837961 | 856 | 12 | |
| 856_12 | Peak_097 | | | 6.345684 | 0.842925 | 856 | 12 | |
| 856_12 | Peak_074 | | | 6.309589 | 0.804188 | 856 | 12 | |
| 856_12 | Peak_411 | | | 5.898485 | 0.84851 | 856 | 12 | |
| 856_12 | Peak_105 | | | 5.87833 | 0.779857 | 856 | 12 | |
| 856_12 | Peak_364 | | | 5.84907 | 0.862529 | 856 | 12 | |
| 856_12 | Peak_323 | | | 5.728819 | 0.792061 | 856 | 12 | |
| 856_12 | Peak_298 | | | 5.690545 | 0.822247 | 856 | 12 | |
| 856_12 | Peak_412 | | | 5.600812 | 0.554644 | 856 | 12 | |
| 856_12 | Peak_001 | | | 5.029414 | 0.851999 | 856 | 12 | |
| 856_12 | Peak_399 | | | 5.006294 | 0.743538 | 856 | 12 | |
| 856_12 | Peak_288 | | | 4.981758 | 0.837964 | 856 | 12 | |
| 856_12 | Peak_398 | | | 4.967805 | 0.903988 | 856 | 12 | |
| 856_12 | Peak_116 | | | 4.904705 | 0.8084 | 856 | 12 | |
| 856_12 | Peak_087 | | | 4.876033 | 0.810744 | 856 | 12 | |
| 856_12 | Peak_056 | | | 4.761242 | 0.796229 | 856 | 12 | |
| 856_12 | Peak_246 | | | 4.753879 | 0.813611 | 856 | 12 | |
| 856_12 | Peak_120 | | | 4.695157 | 0.77271 | 856 | 12 | |
| 856_12 | Peak_278 | | | 4.684386 | 0.8564 | 856 | 12 | |
| 856_12 | Peak_023 | | | 4.676312 | 0.788127 | 856 | 12 | |
| 856_12 | Peak_227 | | | 4.66418 | 0.826151 | 856 | 12 | |
| 856_12 | Peak_391 | | | 4.662337 | 0.905837 | 856 | 12 | |
| 856_12 | Peak_136 | | | 4.649067 | 0.800126 | 856 | 12 | |
| 856_12 | Peak_384 | | | 4.616856 | 0.851035 | 856 | 12 | |
| 856_12 | Peak_092 | | | 4.605551 | 0.821533 | 856 | 12 | |
| 856_12 | Peak_139 | | | 4.594774 | 0.757082 | 856 | 12 | |
| 856_12 | Peak_260 | | | 4.518915 | 0.784746 | 856 | 12 | |
| 856_12 | Peak_306 | | | 4.49259 | 0.831496 | 856 | 12 | |
| 856_12 | Peak_385 | | | 4.442708 | 0.796454 | 856 | 12 | |
| 856_12 | Peak_363 | | | 4.440626 | 0.760357 | 856 | 12 | |
| 856_12 | Peak_005 | | | 4.344349 | 0.852244 | 856 | 12 | |
| 856_12 | Peak_324 | | | 4.334 | 0.849453 | 856 | 12 | |
| 856_12 | Peak_081 | | | 4.28848 | 0.837444 | 856 | 12 | |
| 856_12 | Peak_107 | | | 4.276406 | 0.695715 | 856 | 12 | |
| 856_12 | Peak_155 | | | 4.188868 | 0.753892 | 856 | 12 | |
| 856_12 | Peak_309 | | | 4.023942 | 0.795075 | 856 | 12 | |
| 856_12 | Peak_237 | | | 4.007001 | 0.794319 | 856 | 12 | |
| 856_12 | Peak_103 | | | 3.976037 | 0.853673 | 856 | 12 | |
| 856_12 | Peak_387 | | | 3.968794 | 0.717805 | 856 | 12 | |
| 856_12 | Peak_109 | | | 3.928907 | 0.83001 | 856 | 12 | |
| 856_12 | Peak_313 | | | 3.869333 | 0.792528 | 856 | 12 | |
| 856_12 | Peak_134 | | | 3.831923 | 0.752253 | 856 | 12 | |
| 856_12 | Peak_067 | | | 3.816918 | 0.87625 | 856 | 12 | |
| 856_12 | Peak_226 | | | 3.722454 | 0.771019 | 856 | 12 | |
| 856_12 | Peak_361 | | | 3.711604 | 0.833538 | 856 | 12 | |
| 856_12 | Peak_229 | | | 3.676201 | 0.670045 | 856 | 12 | |
| 856_12 | Peak_316 | | | 3.58067 | 0.704066 | 856 | 12 | |
| 856_12 | Peak_383 | | | 3.465438 | 0.645645 | 856 | 12 | |
| 856_12 | Peak_397 | | | 3.462171 | 0.686562 | 856 | 12 | |
| 856_12 | Peak_198 | | | 3.439385 | 0.749357 | 856 | 12 | |
| 856_12 | Peak_421 | | | 3.436735 | 0.669143 | 856 | 12 | |
| 856_12 | Peak_340 | | | 3.381515 | 0.835973 | 856 | 12 | |
| 856_12 | Peak_021 | | | 3.371492 | 0.859004 | 856 | 12 | |
| 856_12 | Peak_300 | | | 3.326658 | 0.721866 | 856 | 12 | |
| 856_12 | Peak_100 | | | 3.306962 | 0.896436 | 856 | 12 | |
| 856_12 | Peak_277 | | | 3.25468 | 0.747571 | 856 | 12 | |
| 856_12 | Peak_212 | | | 3.252978 | 0.778876 | 856 | 12 | |
| 856_12 | Peak_145 | | | 3.221439 | 0.714349 | 856 | 12 | |
| 856_12 | Peak_390 | | | 3.202083 | 0.748975 | 856 | 12 | |
| 856_12 | Peak_348 | | | 3.199775 | 0.775917 | 856 | 12 | |
| 856_12 | Peak_066 | | | 3.196455 | 0.729877 | 856 | 12 | |
| 856_12 | Peak_333 | | | 3.18364 | 0.747725 | 856 | 12 | |
| 856_12 | Peak_118 | | | 3.1687 | 0.859054 | 856 | 12 | |
| 856_12 | Peak_151 | | | 3.156879 | 0.760011 | 856 | 12 | |
| 856_12 | Peak_367 | | | 3.121297 | 0.751691 | 856 | 12 | |
| 856_12 | Peak_290 | | | 3.093553 | 0.79666 | 856 | 12 | |
| 856_12 | Peak_307 | | | 3.086341 | 0.906879 | 856 | 12 | |
| 856_12 | Peak_330 | | | 3.015362 | 0.744593 | 856 | 12 | |
| 856_12 | Peak_199 | | | 2.992371 | 0.705474 | 856 | 12 | |
| 856_12 | Peak_366 | | | 2.980184 | 0.774519 | 856 | 12 | |
| 856_12 | Peak_326 | | | 2.961035 | 0.815224 | 856 | 12 | |
| 856_12 | Peak_362 | | | 2.957241 | 0.827449 | 856 | 12 | |
| 856_12 | Peak_338 | | | 2.924091 | 0.796576 | 856 | 12 | |
| 856_12 | Peak_112 | | | 2.891929 | 0.728339 | 856 | 12 | |
| 856_12 | Peak_287 | | | 2.850421 | 0.895427 | 856 | 12 | |
| 856_12 | Peak_147 | | | 2.839279 | 0.774119 | 856 | 12 | |
| 856_12 | Peak_351 | | | 2.839009 | 0.749685 | 856 | 12 | |
| 856_12 | Peak_240 | | | 2.81715 | 0.73646 | 856 | 12 | |
| 856_12 | Peak_194 | | | 2.799164 | 0.793103 | 856 | 12 | |
| 856_12 | Peak_130 | | | 2.797135 | 0.767669 | 856 | 12 | |
| 856_12 | Peak_223 | | | 2.78285 | 0.644874 | 856 | 12 | |
| 856_12 | Peak_138 | | | 2.775274 | 0.781697 | 856 | 12 | |
| 856_12 | Peak_211 | | | 2.74872 | 0.877942 | 856 | 12 | |
| 856_12 | Peak_381 | | | 2.733899 | 0.873852 | 856 | 12 | |
| 856_12 | Peak_115 | | | 2.720127 | 0.816696 | 856 | 12 | |
| 856_12 | Peak_179 | | | 2.715156 | 0.644541 | 856 | 12 | |
| 856_12 | Peak_311 | | | 2.679586 | 0.812405 | 856 | 12 | |
| 856_12 | Peak_356 | | | 2.676273 | 0.823748 | 856 | 12 | |
| 856_12 | Peak_205 | | | 2.670357 | 0.671416 | 856 | 12 | |
| 856_12 | Peak_315 | | | 2.639831 | 0.843584 | 856 | 12 | |
| 856_12 | Peak_026 | | | 2.62421 | 0.804789 | 856 | 12 | |
| 856_12 | Peak_261 | | | 2.623256 | 0.720411 | 856 | 12 | |
| 856_12 | Peak_178 | | | 2.61442 | 0.734123 | 856 | 12 | |
| 856_12 | Peak_332 | | | 2.575595 | 0.77241 | 856 | 12 | |
| 856_12 | Peak_342 | | | 2.574216 | 0.842252 | 856 | 12 | |
| 856_12 | Peak_083 | | | 2.565733 | 0.852681 | 856 | 12 | |
| 856_12 | Peak_319 | | | 2.556815 | 0.643412 | 856 | 12 | |
| 856_12 | Peak_380 | | | 2.522811 | 0.670512 | 856 | 12 | |
| 856_12 | Peak_208 | | | 2.517375 | 0.831553 | 856 | 12 | |
| 856_12 | Peak_336 | | | 2.5112 | 0.822109 | 856 | 12 | |
| 856_12 | Peak_281 | | | 2.505649 | 0.824511 | 856 | 12 | |
| 856_12 | Peak_295 | | | 2.498422 | 0.771657 | 856 | 12 | |
| 856_12 | Peak_314 | | | 2.485989 | 0.829951 | 856 | 12 | |
| 856_12 | Peak_070 | | | 2.482967 | 0.829897 | 856 | 12 | |
| 856_12 | Peak_318 | | | 2.476601 | 0.814686 | 856 | 12 | |
| 856_12 | Peak_148 | | | 2.471386 | 0.670287 | 856 | 12 | |
| 856_12 | Peak_339 | | | 2.452354 | 0.844715 | 856 | 12 | |
| 856_12 | Peak_238 | | | 2.419449 | 0.751867 | 856 | 12 | |
| 856_12 | Peak_175 | | | 2.418629 | 0.703324 | 856 | 12 | |
| 856_12 | Peak_352 | | | 2.404057 | 0.74004 | 856 | 12 | |
| 856_12 | Peak_405 | | | 2.388332 | 0.826276 | 856 | 12 | |
| 856_12 | Peak_204 | | | 2.360357 | 0.688598 | 856 | 12 | |
| 856_12 | Peak_280 | | | 2.341225 | 0.75082 | 856 | 12 | |
| 856_12 | Peak_172 | | | 2.332333 | 0.835262 | 856 | 12 | |
| 856_12 | Peak_245 | | | 2.324751 | 0.711282 | 856 | 12 | |
| 856_12 | Peak_119 | | | 2.320077 | 0.676624 | 856 | 12 | |
| 856_12 | Peak_234 | | | 2.291953 | 0.800329 | 856 | 12 | |
| 856_12 | Peak_349 | | | 2.285535 | 0.725704 | 856 | 12 | |
| 856_12 | Peak_263 | | | 2.281127 | 0.63572 | 856 | 12 | |
| 856_12 | Peak_043 | | | 2.259399 | 0.810734 | 856 | 12 | |
| 856_12 | Peak_129 | | | 2.225504 | 0.755807 | 856 | 12 | |
| 856_12 | Peak_174 | | | 2.22187 | 0.634451 | 856 | 12 | |
| 856_12 | Peak_072 | | | 2.21814 | 0.783007 | 856 | 12 | |
| 856_12 | Peak_302 | | | 2.203329 | 0.778972 | 856 | 12 | |
| 856_12 | Peak_337 | | | 2.178634 | 0.787847 | 856 | 12 | |
| 856_12 | Peak_262 | | | 2.170557 | 0.70865 | 856 | 12 | |
| 856_12 | Peak_196 | | | 2.170031 | 0.607565 | 856 | 12 | |
| 856_12 | Peak_160 | | | 2.111451 | 0.54148 | 856 | 12 | |
| 856_12 | Peak_035 | | | 2.095398 | 0.690451 | 856 | 12 | |
| 856_12 | Peak_009 | | | 2.071461 | 0.607443 | 856 | 12 | |
| 856_12 | Peak_327 | | | 2.066458 | 0.665074 | 856 | 12 | |
| 856_12 | Peak_144 | | | 2.053908 | 0.627821 | 856 | 12 | |
| 856_12 | Peak_058 | | | 2.0488 | 0.816269 | 856 | 12 | |
| 856_12 | Peak_382 | | | 2.043735 | 0.807075 | 856 | 12 | |
| 856_12 | Peak_331 | | | 2.040238 | 0.770397 | 856 | 12 | |
| 856_12 | Peak_358 | | | 2.037487 | 0.738751 | 856 | 12 | |
| 856_12 | Peak_095 | | | 2.033801 | 0.660111 | 856 | 12 | |
| 856_12 | Peak_297 | | | 2.024203 | 0.636986 | 856 | 12 | |
| 856_12 | Peak_030 | | | 2.001006 | 0.708333 | 856 | 12 | |
| 856_12 | Peak_335 | | | 1.996803 | 0.903297 | 856 | 12 | |
| 856_12 | Peak_251 | | | 1.991937 | 0.705344 | 856 | 12 | |
| 856_12 | Peak_239 | | | 1.972882 | 0.735124 | 856 | 12 | |
| 856_12 | Peak_149 | | | 1.9713 | 0.800465 | 856 | 12 | |
| 856_12 | Peak_255 | | | 1.952522 | 0.691987 | 856 | 12 | |
| 856_12 | Peak_305 | | | 1.945605 | 0.754874 | 856 | 12 | |
| 856_12 | Peak_171 | | | 1.945027 | 0.532663 | 856 | 12 | |
| 856_12 | Peak_346 | | | 1.943529 | 0.87508 | 856 | 12 | |
| 856_12 | Peak_283 | | | 1.940496 | 0.758113 | 856 | 12 | |
| 856_12 | Peak_406 | | | 1.93229 | 0.746367 | 856 | 12 | |
| 856_12 | Peak_085 | | | 1.921583 | 0.738486 | 856 | 12 | |
| 856_12 | Peak_328 | | | 1.919828 | 0.725944 | 856 | 12 | |
| 856_12 | Peak_228 | | | 1.902681 | 0.806116 | 856 | 12 | |
| 856_12 | Peak_154 | | | 1.901687 | 0.535995 | 856 | 12 | |
| 856_12 | Peak_334 | | | 1.880005 | 0.775152 | 856 | 12 | |
| 856_12 | Peak_308 | | | 1.852982 | 0.559256 | 856 | 12 | |
| 856_12 | Peak_108 | | | 1.84763 | 0.727821 | 856 | 12 | |
| 856_12 | Peak_375 | | | 1.846408 | 0.522299 | 856 | 12 | |
| 856_12 | Peak_247 | | | 1.837303 | 0.704327 | 856 | 12 | |
| 856_12 | Peak_273 | | | 1.83259 | 0.620608 | 856 | 12 | |
| 856_12 | Peak_010 | | | 1.813686 | 0.685753 | 856 | 12 | |
| 856_12 | Peak_202 | | | 1.810391 | 0.813536 | 856 | 12 | |
| 856_12 | Peak_192 | | | 1.789131 | 0.767001 | 856 | 12 | |
| 856_12 | Peak_099 | | | 1.789071 | 0.66243 | 856 | 12 | |
| 856_12 | Peak_065 | | | 1.786848 | 0.856206 | 856 | 12 | |
| 856_12 | Peak_091 | | | 1.778521 | 0.715701 | 856 | 12 | |
| 856_12 | Peak_235 | | | 1.777959 | 0.737285 | 856 | 12 | |
| 856_12 | Peak_289 | | | 1.772691 | 0.653137 | 856 | 12 | |
| 856_12 | Peak_123 | | | 1.772576 | 0.623608 | 856 | 12 | |
| 856_12 | Peak_222 | | | 1.756879 | 0.616452 | 856 | 12 | |
| 856_12 | Peak_173 | | | 1.753843 | 0.571673 | 856 | 12 | |
| 856_12 | Peak_195 | | | 1.731716 | 0.743356 | 856 | 12 | |
| 856_12 | Peak_372 | | | 1.71224 | 0.697425 | 856 | 12 | |
| 856_12 | Peak_146 | | | 1.709535 | 0.62885 | 856 | 12 | |
| 856_12 | Peak_167 | | | 1.704651 | 0.655888 | 856 | 12 | |
| 856_12 | Peak_191 | | | 1.702995 | 0.684671 | 856 | 12 | |
| 856_12 | Peak_317 | | | 1.684678 | 0.647015 | 856 | 12 | |
| 856_12 | Peak_048 | | | 1.683579 | 0.766604 | 856 | 12 | |
| 856_12 | Peak_135 | | | 1.678827 | 0.77552 | 856 | 12 | |
| 856_12 | Peak_369 | | | 1.671088 | 0.703429 | 856 | 12 | |
| 856_12 | Peak_233 | | | 1.654336 | 0.685279 | 856 | 12 | |
| 856_12 | Peak_060 | | | 1.640443 | 0.684496 | 856 | 12 | |
| 856_12 | Peak_177 | | | 1.6374 | 0.577672 | 856 | 12 | |
| 856_12 | Peak_321 | | | 1.62917 | 0.659107 | 856 | 12 | |
| 856_12 | Peak_213 | | | 1.617597 | 0.642483 | 856 | 12 | |
| 856_12 | Peak_114 | | | 1.612276 | 0.697402 | 856 | 12 | |
| 856_12 | Peak_143 | | | 1.612181 | 0.62164 | 856 | 12 | |
| 856_12 | Peak_285 | | | 1.6076 | 0.822074 | 856 | 12 | |
| 856_12 | Peak_230 | | | 1.602919 | 0.701314 | 856 | 12 | |
| 856_12 | Peak_046 | | | 1.595886 | 0.710997 | 856 | 12 | |
| 856_12 | Peak_106 | | | 1.589856 | 0.799909 | 856 | 12 | |
| 856_12 | Peak_292 | | | 1.58199 | 0.639269 | 856 | 12 | |
| 856_12 | Peak_006 | | | 1.580824 | 0.665443 | 856 | 12 | |
| 856_12 | Peak_357 | | | 1.574305 | 0.669694 | 856 | 12 | |
| 856_12 | Peak_410 | | | 1.564873 | 0.564027 | 856 | 12 | |
| 856_12 | Peak_054 | | | 1.563825 | 0.579788 | 856 | 12 | |
| 856_12 | Peak_344 | | | 1.52159 | 0.752903 | 856 | 12 | |
| 856_12 | Peak_102 | | | 1.516776 | 0.730183 | 856 | 12 | |
| 856_12 | Peak_180 | | | 1.510384 | 0.762872 | 856 | 12 | |
| 856_12 | Peak_181 | | | 1.500229 | 0.608493 | 856 | 12 | |
| 856_12 | Peak_039 | | | 1.495346 | 0.743053 | 856 | 12 | |
| 856_12 | Peak_215 | | | 1.493358 | 0.514811 | 856 | 12 | |
| 856_12 | Peak_214 | | | 1.491397 | 0.725898 | 856 | 12 | |
| 856_12 | Peak_022 | | | 1.48834 | 0.727718 | 856 | 12 | |
| 856_12 | Peak_033 | | | 1.484242 | 0.697538 | 856 | 12 | |
| 856_12 | Peak_073 | | | 1.483913 | 0.733361 | 856 | 12 | |
| 856_12 | Peak_232 | | | 1.476427 | 0.666911 | 856 | 12 | |
| 856_12 | Peak_036 | | | 1.472585 | 0.716927 | 856 | 12 | |
| 856_12 | Peak_396 | | | 1.468787 | 0.659053 | 856 | 12 | |
| 856_12 | Peak_373 | | | 1.4613 | 0.582727 | 856 | 12 | |
| 856_12 | Peak_140 | | | 1.451849 | 0.720942 | 856 | 12 | |
| 856_12 | Peak_274 | | | 1.439418 | 0.782244 | 856 | 12 | |
| 856_12 | Peak_077 | | | 1.431008 | 0.715961 | 856 | 12 | |
| 856_12 | Peak_347 | | | 1.417015 | 0.792043 | 856 | 12 | |
| 856_12 | Peak_206 | | | 1.414052 | 0.660646 | 856 | 12 | |
| 856_12 | Peak_117 | | | 1.401153 | 0.759756 | 856 | 12 | |
| 856_12 | Peak_157 | | | 1.400216 | 0.523768 | 856 | 12 | |
| 856_12 | Peak_301 | | | 1.394701 | 0.624497 | 856 | 12 | |
| 856_12 | Peak_076 | | | 1.391091 | 0.733367 | 856 | 12 | |
| 856_12 | Peak_378 | | | 1.389749 | 0.765147 | 856 | 12 | |
| 856_12 | Peak_188 | | | 1.381076 | 0.659396 | 856 | 12 | |
| 856_12 | Peak_325 | | | 1.38026 | 0.709404 | 856 | 12 | |
| 856_12 | Peak_296 | | | 1.370431 | 0.683623 | 856 | 12 | |
| 856_12 | Peak_374 | | | 1.359609 | 0.733438 | 856 | 12 | |
| 856_12 | Peak_051 | | | 1.345071 | 0.698112 | 856 | 12 | |
| 856_12 | Peak_044 | | | 1.342111 | 0.818633 | 856 | 12 | |
| 856_12 | Peak_256 | | | 1.339892 | 0.70144 | 856 | 12 | |
| 856_12 | Peak_282 | | | 1.333924 | 0.609455 | 856 | 12 | |
| 856_12 | Peak_127 | | | 1.327727 | 0.716577 | 856 | 12 | |
| 856_12 | Peak_141 | | | 1.326778 | 0.735482 | 856 | 12 | |
| 856_12 | Peak_310 | | | 1.319852 | 0.718572 | 856 | 12 | |
| 856_12 | Peak_203 | | | 1.313291 | 0.649511 | 856 | 12 | |
| 856_12 | Peak_137 | | | 1.30204 | 0.758969 | 856 | 12 | |
| 856_12 | Peak_368 | | | 1.300111 | 0.638933 | 856 | 12 | |
| 856_12 | Peak_304 | | | 1.298186 | 0.722829 | 856 | 12 | |
| 856_12 | Peak_224 | | | 1.297079 | 0.582825 | 856 | 12 | |
| 856_12 | Peak_359 | | | 1.296185 | 0.69805 | 856 | 12 | |
| 856_12 | Peak_041 | | | 1.295688 | 0.634809 | 856 | 12 | |
| 856_12 | Peak_341 | | | 1.293757 | 0.697962 | 856 | 12 | |
| 856_12 | Peak_303 | | | 1.287433 | 0.618996 | 856 | 12 | |
| 856_12 | Peak_075 | | | 1.280713 | 0.581027 | 856 | 12 | |
| 856_12 | Peak_329 | | | 1.27415 | 0.689746 | 856 | 12 | |
| 856_12 | Peak_024 | | | 1.268756 | 0.656297 | 856 | 12 | |
| 856_12 | Peak_028 | | | 1.265662 | 0.634483 | 856 | 12 | |
| 856_12 | Peak_216 | | | 1.261462 | 0.689222 | 856 | 12 | |
| 856_12 | Peak_121 | | | 1.258899 | 0.634785 | 856 | 12 | |
| 856_12 | Peak_098 | | | 1.258186 | 0.683887 | 856 | 12 | |
| 856_12 | Peak_379 | | | 1.251023 | 0.68448 | 856 | 12 | |
| 856_12 | Peak_291 | | | 1.249863 | 0.540849 | 856 | 12 | |
| 856_12 | Peak_201 | | | 1.248441 | 0.744932 | 856 | 12 | |
| 856_12 | Peak_299 | | | 1.246143 | 0.625074 | 856 | 12 | |
| 856_12 | Peak_122 | | | 1.230115 | 0.706815 | 856 | 12 | |
| 856_12 | Peak_104 | | | 1.220047 | 0.806167 | 856 | 12 | |
| 856_12 | Peak_241 | | | 1.210278 | 0.663274 | 856 | 12 | |
| 856_12 | Peak_320 | | | 1.209623 | 0.62459 | 856 | 12 | |
| 856_12 | Peak_093 | | | 1.206778 | 0.580072 | 856 | 12 | |
| 856_12 | Peak_062 | | | 1.204767 | 0.701586 | 856 | 12 | |
| 856_12 | Peak_210 | | | 1.198679 | 0.626736 | 856 | 12 | |
| 856_12 | Peak_345 | | | 1.193714 | 0.68785 | 856 | 12 | |
| 856_12 | Peak_231 | | | 1.184916 | 0.675776 | 856 | 12 | |
| 856_12 | Peak_312 | | | 1.168055 | 0.61686 | 856 | 12 | |
| 856_12 | Peak_018 | | | 1.166266 | 0.50241 | 856 | 12 | |
| 856_12 | Peak_236 | | | 1.165877 | 0.65052 | 856 | 12 | |
| 856_12 | Peak_207 | | | 1.156755 | 0.679782 | 856 | 12 | |
| 856_12 | Peak_031 | | | 1.155783 | 0.472189 | 856 | 12 | |
| 856_12 | Peak_142 | | | 1.147176 | 0.626484 | 856 | 12 | |
| 856_12 | Peak_159 | | | 1.141102 | 0.511961 | 856 | 12 | |
| 856_12 | Peak_068 | | | 1.140433 | 0.763551 | 856 | 12 | |
| 856_12 | Peak_350 | | | 1.136385 | 0.641193 | 856 | 12 | |
| 856_12 | Peak_132 | | | 1.130249 | 0.705156 | 856 | 12 | |
| 856_12 | Peak_322 | | | 1.128504 | 0.683141 | 856 | 12 | |
| 856_12 | Peak_242 | | | 1.119709 | 0.595654 | 856 | 12 | |
| 856_12 | Peak_063 | | | 1.110888 | 0.461258 | 856 | 12 | |
| 856_12 | Peak_294 | | | 1.105321 | 0.719948 | 856 | 12 | |
| 856_12 | Peak_089 | | | 1.083024 | 0.58914 | 856 | 12 | |
| 856_12 | Peak_086 | | | 1.069928 | 0.667489 | 856 | 12 | |
| 856_12 | Peak_008 | | | 1.063774 | 0.580357 | 856 | 12 | |
| 856_12 | Peak_267 | | | 1.041183 | 0.445171 | 856 | 12 | |
| 856_12 | Peak_200 | | | 1.031012 | 0.523622 | 856 | 12 | |
| 856_12 | Peak_096 | | | 1.025376 | 0.39559 | 856 | 12 | |
| 856_12 | Peak_057 | | | 1.017349 | 0.801121 | 856 | 12 | |
| 856_12 | Peak_243 | | | 1.011945 | 0.545777 | 856 | 12 | |
| 856_12 | Peak_360 | | | 0.978735 | 0.601084 | 856 | 12 | |
| 856_12 | Peak_113 | | | 0.949837 | 0.647795 | 856 | 12 | |
| 856_12 | Peak_111 | | | 0.935181 | 0.595329 | 856 | 12 | |
| 856_12 | Peak_110 | | | 0.921323 | 0.592513 | 856 | 12 | |
| 856_12 | Peak_055 | | | 0.916981 | 0.384034 | 856 | 12 | |
| 856_12 | Peak_131 | | | 0.910891 | 0.617751 | 856 | 12 | |
| 856_12 | Peak_052 | | | 0.909328 | 0.489266 | 856 | 12 | |
| 856_12 | Peak_090 | | | 0.894044 | 0.688367 | 856 | 12 | |
| 856_12 | Peak_003 | | | 0.880279 | 0.570067 | 856 | 12 | |
| 856_12 | Peak_016 | | | 0.879545 | 0.566241 | 856 | 12 | |
| 856_12 | Peak_408 | | | 0.813148 | 0.419589 | 856 | 12 | |
| 856_12 | Peak_218 | | | 0.725641 | 0.414433 | 856 | 12 | |
| 856_12 | Peak_286 | | | 0.723861 | 0.557911 | 856 | 12 | |
| 856_12 | Peak_258 | | | 0.66851 | 0.527048 | 856 | 12 | |
| 856_12 | Peak_193 | | | 0.619689 | 0.488643 | 856 | 12 | |

TABLE 4
Group of different fragment ions from precursor m/z 857.7472 corresponding
to ionized TAG (C16:0; C18:1; C18:3) found in the SWATH ® window m/z 857

| UnitMassFile_R | Name | Cluster | Group_FSD | m.z | RT | m.z. Min | m.z. Max | RT. Min | RT. Max |
|---|---|---|---|---|---|---|---|---|---|
| 857_5 | Peak_121 | 84 | 147 | 600.5065 | 3.853865 | 600.4633 | 600.5567 | 3.779129 | 3.931176 |
| 857_5 | Peak_122 | 85 | 147 | 601.5084 | 3.864302 | 601.4668 | 601.5568 | 3.806114 | 3.987033 |
| 857_5 | Peak_141 | 96 | 147 | 857.7481 | 3.855109 | 857.6902 | 857.81 | 3.806114 | 3.916569 |
| 857_5 | Peak_107 | 4 | 147 | 544.4798 | 3.854616 | 544.435 | 544.5174 | 3.792988 | 3.931176 |
| 857_5 | Peak_003 | 9 | 147 | 95.0864 | 3.8571 | 95.07782 | 95.09571 | 3.806114 | 3.973527 |
| 857_5 | Peak_103 | 72 | 147 | 526.4694 | 3.853787 | 526.4356 | 526.5068 | 3.792988 | 3.916569 |
| 857_5 | Peak_069 | 2 | 147 | 314.2769 | 3.855135 | 314.257 | 314.2995 | 3.806114 | 3.916569 |
| 857_5 | Peak_010 | 16 | 147 | 109.1015 | 3.858516 | 109.0931 | 109.1108 | 3.806114 | 3.987033 |
| 857_5 | Peak_048 | 51 | 147 | 261.2214 | 3.855978 | 261.2071 | 261.2391 | 3.806114 | 3.916569 |
| 857_5 | Peak_108 | 4 | 147 | 545.4807 | 3.862852 | 545.4499 | 545.5159 | 3.806114 | 3.996304 |
| 857_5 | Peak_120 | 83 | 147 | 599.5027 | 3.853707 | 599.4641 | 599.5436 | 3.792988 | 3.916569 |
| 857_5 | Peak_104 | 73 | 147 | 527.4719 | 3.869873 | 527.44 | 527.5113 | 3.820896 | 3.987033 |
| 857_5 | Peak_067 | 59 | 147 | 313.2739 | 3.860969 | 313.2544 | 313.2943 | 3.806114 | 3.973527 |
| 857_5 | Peak_049 | 1 | 147 | 262.2243 | 3.855187 | 262.2096 | 262.2416 | 3.806114 | 3.916569 |
| 857_5 | Peak_013 | 19 | 147 | 121.1013 | 3.856685 | 121.093 | 121.1117 | 3.806114 | 3.945292 |
| 857_5 | Peak_018 | 24 | 147 | 135.1168 | 3.855039 | 135.1083 | 135.1263 | 3.820896 | 3.916569 |
| 857_5 | Peak_002 | 8 | 147 | 93.07082 | 3.856575 | 93.06398 | 93.07896 | 3.820896 | 3.916569 |
| 857_5 | Peak_131 | 93 | 147 | 856.7451 | 3.853788 | 856.6942 | 856.7933 | 3.806114 | 3.916569 |
| 857_5 | Peak_005 | 11 | 147 | 97.10152 | 3.857403 | 97.09375 | 97.10905 | 3.806114 | 3.959776 |
| 857_5 | Peak_012 | 18 | 147 | 119.0856 | 3.854038 | 119.0776 | 119.0945 | 3.806114 | 3.916569 |
| 857_5 | Peak_014 | 20 | 147 | 123.1167 | 3.856174 | 123.1082 | 123.1254 | 3.820896 | 3.945292 |
| 857_5 | Peak_102 | 71 | 147 | 525.4655 | 3.854769 | 525.4321 | 525.5 | 3.806114 | 3.916569 |
| 857_5 | Peak_008 | 14 | 147 | 107.086 | 3.855535 | 107.0783 | 107.0944 | 3.820896 | 3.916569 |
| 857_5 | Peak_004 | 10 | 147 | 96.08921 | 3.853207 | 96.08245 | 96.09767 | 3.806114 | 3.916569 |
| 857_5 | Peak_106 | 74 | 147 | 543.4762 | 3.854408 | 543.4507 | 543.5099 | 3.806114 | 3.916569 |
| 857_5 | Peak_023 | 29 | 147 | 149.1321 | 3.855808 | 149.1228 | 149.1417 | 3.820896 | 3.916569 |
| 857_5 | Peak_030 | 35 | 147 | 175.1476 | 3.855186 | 175.1376 | 175.1582 | 3.806114 | 3.916569 |
| 857_5 | Peak_015 | 21 | 147 | 131.0853 | 3.856111 | 131.0764 | 131.0942 | 3.820896 | 3.916569 |
| 857_5 | Peak_007 | 13 | 147 | 105.0703 | 3.855682 | 105.0634 | 105.0779 | 3.820896 | 3.916569 |
| 857_5 | Peak_029 | 34 | 147 | 173.1325 | 3.857111 | 173.1223 | 173.1427 | 3.820896 | 3.916569 |
| 857_5 | Peak_016 | 22 | 147 | 133.1009 | 3.855251 | 133.0929 | 133.1091 | 3.820896 | 3.916569 |
| 857_5 | Peak_037 | 41 | 147 | 187.1477 | 3.854365 | 187.1386 | 187.1579 | 3.820896 | 3.916569 |
| 857_5 | Peak_044 | 47 | 147 | 243.2105 | 3.856424 | 243.1984 | 243.2226 | 3.806114 | 3.916569 |
| 857_5 | Peak_020 | 26 | 147 | 145.1011 | 3.855794 | 145.0928 | 145.1115 | 3.806114 | 3.916569 |
| 857_5 | Peak_071 | 60 | 147 | 317.2471 | 3.855527 | 317.2342 | 317.2618 | 3.820896 | 3.916569 |
| 857_5 | Peak_026 | 32 | 147 | 161.1323 | 3.857545 | 161.1245 | 161.1424 | 3.820896 | 3.916569 |
| 857_5 | Peak_027 | 33 | 147 | 163.1474 | 3.854831 | 163.1393 | 163.1573 | 3.806114 | 3.916569 |
| 857_5 | Peak_038 | 42 | 147 | 189.1633 | 3.857163 | 189.1541 | 189.1755 | 3.820896 | 3.916569 |
| 857_5 | Peak_130 | 92 | 147 | 839.7337 | 3.855717 | 839.6998 | 839.7571 | 3.820896 | 3.916569 |
| 857_5 | Peak_045 | 48 | 147 | 244.2137 | 3.855538 | 244.2032 | 244.2253 | 3.806114 | 3.90101 |
| 857_5 | Peak_072 | 3 | 147 | 318.2505 | 3.854408 | 318.2381 | 318.2658 | 3.820896 | 3.916569 |
| 857_5 | Peak_070 | 2 | 147 | 315.2777 | 3.855114 | 315.2587 | 315.2963 | 3.806114 | 3.90101 |
| 857_5 | Peak_124 | 87 | 147 | 618.5165 | 3.854487 | 618.4893 | 618.5455 | 3.820896 | 3.916569 |
| 857_5 | Peak_022 | 28 | 147 | 147.116 | 3.85589 | 147.1077 | 147.1248 | 3.820896 | 3.916569 |
| 857_5 | Peak_040 | 44 | 147 | 201.1633 | 3.856308 | 201.1536 | 201.1736 | 3.820896 | 3.916569 |
| 857_5 | Peak_019 | 25 | 147 | 137.1324 | 3.856603 | 137.124 | 137.1422 | 3.820896 | 3.945292 |
| 857_5 | Peak_043 | 46 | 147 | 239.2364 | 3.859123 | 239.2261 | 239.2479 | 3.820896 | 3.916569 |
| 857_5 | Peak_117 | 81 | 147 | 577.5126 | 3.870045 | 577.4819 | 577.543 | 3.820896 | 3.987033 |
| 857_5 | Peak_025 | 31 | 147 | 159.1164 | 3.855445 | 159.108 | 159.1258 | 3.806114 | 3.916569 |
| 857_5 | Peak_065 | 57 | 147 | 289.2521 | 3.854936 | 289.2401 | 289.2665 | 3.820896 | 3.931176 |
| 857_5 | Peak_058 | 55 | 147 | 275.2372 | 3.853372 | 275.2241 | 275.2499 | 3.820896 | 3.916569 |
| 857_5 | Peak_099 | 70 | 147 | 508.4569 | 3.854486 | 508.4362 | 508.4808 | 3.820896 | 3.916569 |
| 857_5 | Peak_123 | 86 | 147 | 617.5066 | 3.854946 | 617.4822 | 617.5313 | 3.820896 | 3.916569 |
| 857_5 | Peak_089 | 63 | 147 | 374.35 | 3.853849 | 374.3376 | 374.3649 | 3.806114 | 3.90101 |
| 857_5 | Peak_042 | 45 | 147 | 215.1789 | 3.857007 | 215.169 | 215.1877 | 3.820896 | 3.916569 |
| 857_5 | Peak_090 | 64 | 147 | 388.365 | 3.854915 | 388.348 | 388.3814 | 3.806114 | 3.90101 |
| 857_5 | Peak_095 | 66 | 147 | 418.3745 | 3.855047 | 418.3585 | 418.3931 | 3.806114 | 3.90101 |
| 857_5 | Peak_115 | 79 | 147 | 575.4954 | 3.872936 | 575.462 | 575.5162 | 3.837129 | 3.973527 |
| 857_5 | Peak_001 | 7 | 147 | 91.05509 | 3.856937 | 91.04879 | 91.06226 | 3.820896 | 3.916569 |
| 857_5 | Peak_066 | 58 | 147 | 290.2554 | 3.854115 | 290.242 | 290.2684 | 3.806114 | 3.90101 |
| 857_5 | Peak_011 | 17 | 147 | 111.1167 | 3.859264 | 111.1089 | 111.1238 | 3.820896 | 3.916569 |
| 857_5 | Peak_050 | 1 | 147 | 263.2308 | 3.859685 | 263.2162 | 263.2437 | 3.820896 | 3.959776 |
| 857_5 | Peak_017 | 23 | 147 | 134.1052 | 3.854308 | 134.0962 | 134.1126 | 3.806114 | 3.90101 |
| 857_5 | Peak_039 | 43 | 147 | 191.1788 | 3.854654 | 191.1707 | 191.1863 | 3.820896 | 3.931176 |
| 857_5 | Peak_036 | 40 | 147 | 185.132 | 3.857314 | 185.1243 | 185.1416 | 3.820896 | 3.916569 |
| 857_5 | Peak_024 | 30 | 147 | 150.1357 | 3.850694 | 150.1294 | 150.1432 | 3.806114 | 3.90101 |
| 857_5 | Peak_046 | 49 | 147 | 247.2414 | 3.855993 | 247.2323 | 247.2523 | 3.820896 | 3.916569 |
| 857_5_{—} | Peak_094 | 65 | 147 | 414.3795 | 3.855258 | 414.3636 | 414.3952 | 3.820896 | 3.916569 |
| 857_5 | Peak_097 | 68 | 147 | 474.4019 | 3.853651 | 474.3877 | 474.4185 | 3.806114 | 3.90101 |
| 857_5 | Peak_009 | 15 | 147 | 108.0901 | 3.861538 | 108.0826 | 108.0973 | 3.820896 | 3.916569 |
| 857_5 | Peak_088 | 62 | 147 | 373.3444 | 3.85505 | 373.3305 | 373.3605 | 3.820896 | 3.916569 |
| 857_5 | Peak_073 | 3 | 147 | 319.2588 | 3.853882 | 319.2461 | 319.2713 | 3.820896 | 3.916569 |

| | | | | | | | | MS/MS library |
|---|---|---|---|---|---|---|---|---|
| UnitMassFile_R | Name | Library. m.z | Library. RT | Slope | RSQ | UnitMass_File | R ClusterNumber | annotation |
| 857_5 | Peak_121 | | | 24.59012 | 0.738636 | 857 | 5 | |
| 857_5 | Peak_122 | | | 20.03242 | 0.919334 | 857 | 5 | |
| 857_5 | Peak_141 | | | 12.89089 | 0.816797 | 857 | 5 | |
| 857_5 | Peak_107 | | | 11.38678 | 0.775801 | 857 | 5 | |
| 857_5 | Peak_003 | | | 10.3326 | 0.840953 | 857 | 5 | |
| 857_5 | Peak_103 | | | 9.848763 | 0.856285 | 857 | 5 | |
| 857_5 | Peak_069 | | | 7.4653 | 0.841731 | 857 | 5 | |
| 857_5 | Peak_010 | | | 7.452555 | 0.860164 | 857 | 5 | |
| 857_5 | Peak_048 | | | 6.776567 | 0.749804 | 857 | 5 | |
| 857_5 | Peak_108 | | | 6.710198 | 0.838602 | 857 | 5 | |
| 857_5 | Peak_120 | | | 5.940344 | 0.831549 | 857 | 5 | |
| 857_5 | Peak_104 | | | 5.758957 | 0.810386 | 857 | 5 | |
| 857_5 | Peak_067 | | | 5.745161 | 0.845232 | 857 | 5 | |
| 857_5 | Peak_049 | | | 5.244346 | 0.74336 | 857 | 5 | |
| 857_5 | Peak_013 | | | 4.700987 | 0.712047 | 857 | 5 | |
| 857_5 | Peak_018 | | | 3.823503 | 0.743959 | 857 | 5 | |
| 857_5 | Peak_002 | | | 3.742726 | 0.774813 | 857 | 5 | |
| 857_5 | Peak_131 | | | 3.595974 | 0.611535 | 857 | 5 | |
| 857_5 | Peak_005 | | | 3.406505 | 0.753666 | 857 | 5 | |
| 857_5 | Peak_012 | | | 3.335417 | 0.806228 | 857 | 5 | |
| 857_5 | Peak_014 | | | 3.327867 | 0.739949 | 857 | 5 | |
| 857_5 | Peak_102 | | | 3.250158 | 0.774768 | 857 | 5 | |
| 857_5 | Peak_008 | | | 3.15209 | 0.75525 | 857 | 5 | |
| 857_5 | Peak_004 | | | 2.970763 | 0.8135 | 857 | 5 | |
| 857_5 | Peak_106 | | | 2.952595 | 0.55316 | 857 | 5 | |
| 857_5 | Peak_023 | | | 2.770124 | 0.723418 | 857 | 5 | |
| 857_5 | Peak_030 | | | 2.754522 | 0.715183 | 857 | 5 | |
| 857_5 | Peak_015 | | | 2.686689 | 0.744949 | 857 | 5 | |
| 857_5 | Peak_007 | | | 2.655192 | 0.712392 | 857 | 5 | |
| 857_5 | Peak_029 | | | 2.575421 | 0.760437 | 857 | 5 | |
| 857_5 | Peak_016 | | | 2.502975 | 0.704614 | 857 | 5 | |
| 857_5 | Peak_037 | | | 2.495337 | 0.683503 | 857 | 5 | |
| 857_5 | Peak_044 | | | 2.490581 | 0.670963 | 857 | 5 | |
| 857_5 | Peak_020 | | | 2.4271 | 0.775475 | 857 | 5 | |
| 857_5 | Peak_071 | | | 2.358419 | 0.645596 | 857 | 5 | |
| 857_5 | Peak_026 | | | 2.246181 | 0.73681 | 857 | 5 | |
| 857_5 | Peak_027 | | | 2.235858 | 0.659204 | 857 | 5 | |
| 857_5 | Peak_038 | | | 2.177453 | 0.762381 | 857 | 5 | |
| 857_5 | Peak_130 | | | 2.16159 | 0.784353 | 857 | 5 | |
| 857_5 | Peak_045 | | | 2.069586 | 0.821832 | 857 | 5 | |
| 857_5 | Peak_072 | | | 1.937926 | 0.754805 | 857 | 5 | |
| 857_5 | Peak_070 | | | 1.867404 | 0.818749 | 857 | 5 | |
| 857_5 | Peak_124 | | | 1.852025 | 0.772925 | 857 | 5 | |
| 857_5 | Peak_022 | | | 1.829422 | 0.718943 | 857 | 5 | |
| 857_5 | Peak_040 | | | 1.737172 | 0.76229 | 857 | 5 | |
| 857_5 | Peak_019 | | | 1.722423 | 0.737299 | 857 | 5 | |
| 857_5 | Peak_043 | | | 1.688293 | 0.678331 | 857 | 5 | |
| 857_5 | Peak_117 | | | 1.687575 | 0.542912 | 857 | 5 | |
| 857_5 | Peak_025 | | | 1.668663 | 0.750823 | 857 | 5 | |
| 857_5 | Peak_065 | | | 1.617158 | 0.711122 | 857 | 5 | |
| 857_5 | Peak_058 | | | 1.60807 | 0.740808 | 857 | 5 | |
| 857_5 | Peak_099 | | | 1.566999 | 0.667738 | 857 | 5 | |
| 857_5 | Peak_123 | | | 1.562638 | 0.6493 | 857 | 5 | |
| 857_5 | Peak_089 | | | 1.542053 | 0.745287 | 857 | 5 | |
| 857_5 | Peak_042 | | | 1.540027 | 0.86134 | 857 | 5 | |
| 857_5 | Peak_090 | | | 1.523074 | 0.677787 | 857 | 5 | |
| 857_5 | Peak_095 | | | 1.472093 | 0.700874 | 857 | 5 | |
| 857_5 | Peak_115 | | | 1.370637 | 0.708563 | 857 | 5 | |
| 857_5 | Peak_001 | | | 1.339218 | 0.671115 | 857 | 5 | |
| 857_5 | Peak_066 | | | 1.309917 | 0.637163 | 857 | 5 | |
| 857_5 | Peak_011 | | | 1.217225 | 0.640432 | 857 | 5 | |
| 857_5 | Peak_050 | | | 1.18648 | 0.517974 | 857 | 5 | |
| 857_5 | Peak_017 | | | 1.179618 | 0.718501 | 857 | 5 | |
| 857_5 | Peak_039 | | | 1.15176 | 0.591398 | 857 | 5 | |
| 857_5 | Peak_036 | | | 1.102027 | 0.557832 | 857 | 5 | |
| 857_5 | Peak_024 | | | 1.050823 | 0.735873 | 857 | 5 | |
| 857_5 | Peak_046 | | | 1.034943 | 0.382118 | 857 | 5 | |
| 857_5 | Peak_094 | | | 0.973507 | 0.541391 | 857 | 5 | |
| 857_5 | Peak_097 | | | 0.969744 | 0.732268 | 857 | 5 | |
| 857_5 | Peak_009 | | | 0.938989 | 0.549201 | 857 | 5 | |
| 857_5 | Peak_088 | | | 0.908274 | 0.53659 | 857 | 5 | |
| 857_5 | Peak_073 | | | 0.828017 | 0.50458 | 857 | 5 | |

TABLE 5
Group of different fragment ions from precursor m/z 872.7689 corresponding
to ionized TAG (C16:0; C18:1; C18:3) found in the SWATH ® window m/z 872

| UnitMassFile_R | Name | Cluster | Group_FSD | m.z | RT | m.z. Min | m.z. Max | RT. Min | RT. Max |
|---|---|---|---|---|---|---|---|---|---|
| 872 12 | Peak 34828 | 1696 | 147 | 872.7689 | 3.857361 | 872.7424 | 872.83 | 3.723517 | 3.961833 |
| 872 12 | Peak 76 | 59 | 147 | 575.5035 | 3.836822 | 575.4608 | 575.5455 | 3.753233 | 3.954973 |
| 872 12 | Peak 81 | 64 | 147 | 599.5034 | 3.837732 | 599.4595 | 599.5563 | 3.753233 | 3.944532 |
| 872 12 | Peak 03 | 5 | 147 | 95.08687 | 3.837313 | 95.07757 | 95.0996 | 3.753233 | 3.913017 |
| 872 12 | Peak 46 | 37 | 147 | 263.2376 | 3.836556 | 263.219 | 263.2602 | 3.769067 | 3.913017 |
| 872 12 | Peak 11 | 10 | 147 | 109.1019 | 3.837489 | 109.0929 | 109.1136 | 3.769067 | 3.913017 |
| 872 12 | Peak 77 | 60 | 147 | 576.5061 | 3.834467 | 576.4805 | 576.5347 | 3.753233 | 3.897742 |
| 872 12 | Peak 15 | 14 | 147 | 123.1173 | 3.83679 | 123.108 | 123.1284 | 3.769067 | 3.913017 |
| 872 12 | Peak 41 | 32 | 147 | 239.2373 | 3.839016 | 239.222 | 239.2548 | 3.7849 | 3.913017 |
| 872 12 | Peak 43 | 34 | 147 | 245.2267 | 3.837308 | 245.2104 | 245.2436 | 3.7849 | 3.897742 |
| 872 12 | Peak 14 | 13 | 147 | 121.1016 | 3.836373 | 121.0929 | 121.1115 | 3.769067 | 3.897742 |
| 872 12 | Peak 55 | 42 | 147 | 313.2739 | 3.843347 | 313.2526 | 313.295 | 3.769067 | 3.913017 |
| 872 12 | Peak 10 | 9 | 147 | 107.0864 | 3.837507 | 107.0781 | 107.0956 | 3.769067 | 3.913017 |
| 872 12 | Peak 20 | 18 | 147 | 137.1326 | 3.836117 | 137.1239 | 137.1421 | 3.769067 | 3.897742 |
| 872 12 | Peak 19 | 17 | 147 | 135.1172 | 3.837592 | 135.1082 | 135.1279 | 3.769067 | 3.897742 |
| 872 12 | Peak 02 | 4 | 147 | 93.07117 | 3.837011 | 93.06372 | 93.08007 | 3.769067 | 3.897742 |
| 872 12 | Peak 28 | 25 | 147 | 175.1482 | 3.837718 | 175.1377 | 175.1601 | 3.7849 | 3.913017 |
| 872 12 | Peak 05 | 7 | 147 | 97.1021 | 3.83775 | 97.09489 | 97.1102 | 3.7849 | 3.913017 |
| 872 12 | Peak 61 | 47 | 147 | 337.2737 | 3.836691 | 337.2543 | 337.2957 | 3.769067 | 3.897742 |
| 872 12 | Peak 74 | 57 | 147 | 573.486 | 3.834432 | 573.4512 | 573.5222 | 3.753233 | 3.897742 |
| 872 12 | Peak 80 | 63 | 147 | 577.5176 | 3.846675 | 577.4807 | 577.552 | 3.769067 | 3.897742 |
| 872 12 | Peak 27 | 24 | 147 | 163.1483 | 3.837918 | 163.1392 | 163.1591 | 3.7849 | 3.897742 |
| 872 12 | Peak 18 | 16 | 147 | 133.1014 | 3.837875 | 133.0927 | 133.1123 | 3.7849 | 3.897742 |
| 872 12 | Peak 24 | 21 | 147 | 149.1324 | 3.837585 | 149.1227 | 149.1434 | 3.769067 | 3.897742 |
| 872 12 | Peak 26 | 23 | 147 | 161.1324 | 3.838239 | 161.1227 | 161.1442 | 3.7849 | 3.897742 |
| 872 12 | Peak 21 | 19 | 147 | 147.117 | 3.837735 | 147.1077 | 147.1265 | 3.7849 | 3.897742 |
| 872 12 | Peak 56 | 43 | 147 | 319.2634 | 3.837094 | 319.2469 | 319.2822 | 3.7849 | 3.897742 |
| 872 12 | Peak 82 | 65 | 147 | 601.5165 | 3.82551 | 601.4899 | 601.5487 | 3.769067 | 3.882101 |
| 872 12 | Peak 37 | 29 | 147 | 189.1643 | 3.838616 | 189.1542 | 189.1756 | 3.7849 | 3.897742 |
| 872 12 | Peak 45 | 36 | 147 | 261.2222 | 3.83848 | 261.2098 | 261.2372 | 3.7849 | 3.897742 |
| 872 12 | Peak 25 | 22 | 147 | 151.1477 | 3.837136 | 151.1393 | 151.1567 | 3.7849 | 3.897742 |
| 872 12 | Peak 01 | 3 | 147 | 91.05538 | 3.837304 | 91.04853 | 91.06335 | 3.7849 | 3.913017 |
| 872 12 | Peak 13 | 12 | 147 | 119.086 | 3.838158 | 119.0774 | 119.0959 | 3.7849 | 3.897742 |
| 872 12 | Peak 73 | 56 | 147 | 551.5017 | 3.884979 | 551.4783 | 551.528 | 3.850831 | 3.944532 |
| 872 12 | Peak 29 | 26 | 147 | 177.1635 | 3.838891 | 177.1553 | 177.1741 | 3.802567 | 3.897742 |
| 872 12 | Peak 12 | 11 | 147 | 111.1173 | 3.837992 | 111.1102 | 111.1251 | 3.802567 | 3.897742 |
| 872 12 | Peak 04 | 6 | 147 | 97.06673 | 3.836534 | 97.06013 | 97.07403 | 3.7849 | 3.897742 |
| 872 12 | Peak 42 | 33 | 147 | 243.212 | 3.839974 | 243.2009 | 243.2252 | 3.802567 | 3.897742 |
| 872 12 | Peak 09 | 8 | 147 | 105.0705 | 3.838438 | 105.0632 | 105.0791 | 3.802567 | 3.913017 |
| 872 12 | Peak 38 | 30 | 147 | 203.1791 | 3.848753 | 203.1687 | 203.1888 | 3.802567 | 3.928958 |
| 872 12 | Peak 39 | 31 | 147 | 221.2259 | 3.842237 | 221.2178 | 221.2367 | 3.802567 | 3.897742 |
| 872 12 | Peak 62 | 48 | 147 | 339.2898 | 3.842674 | 339.2792 | 339.3052 | 3.7849 | 3.897742 |

| | | | | | | | | MS/MS library |
|---|---|---|---|---|---|---|---|---|
| UnitMassFile_R | Name | Library. m.z | Library. RT | Slope | RSQ | UnitMass_File | R ClusterNumber | annotation |
| 872 12 | Peak 34828 | | | 278.0476 | 0.917623 | 872 | 12 | |
| 872 12 | Peak 76 | | | 152.478 | 0.90614 | 872 | 12 | |
| 872 12 | Peak 81 | | | 61.75987 | 0.878599 | 872 | 12 | |
| 872 12 | Peak 03 | | | 28.0951 | 0.89284 | 872 | 12 | |
| 872 12 | Peak 46 | | | 18.80244 | 0.872528 | 872 | 12 | |
| 872 12 | Peak 11 | | | 16.27492 | 0.836656 | 872 | 12 | |
| 872 12 | Peak 77 | | | 11.38982 | 0.669656 | 872 | 12 | |
| 872 12 | Peak 15 | | | 11.18957 | 0.822443 | 872 | 12 | |
| 872 12 | Peak 41 | | | 9.282391 | 0.903697 | 872 | 12 | |
| 872 12 | Peak 43 | | | 7.480613 | 0.845043 | 872 | 12 | |
| 872 12 | Peak 14 | | | 7.36471 | 0.823509 | 872 | 12 | |
| 872 12 | Peak 55 | | | 6.469196 | 0.837865 | 872 | 12 | |
| 872 12 | Peak 10 | | | 5.821841 | 0.86684 | 872 | 12 | |
| 872 12 | Peak 20 | | | 5.789719 | 0.869726 | 872 | 12 | |
| 872 12 | Peak 19 | | | 5.578299 | 0.875909 | 872 | 12 | |
| 872 12 | Peak 02 | | | 5.479019 | 0.738026 | 872 | 12 | |
| 872 12 | Peak 28 | | | 5.448532 | 0.894498 | 872 | 12 | |
| 872 12 | Peak 05 | | | 5.299362 | 0.907638 | 872 | 12 | |
| 872 12 | Peak 61 | | | 4.902367 | 0.870585 | 872 | 12 | |
| 872 12 | Peak 74 | | | 4.74638 | 0.850339 | 872 | 12 | |
| 872 12 | Peak 80 | | | 4.596398 | 0.718252 | 872 | 12 | |
| 872 12 | Peak 27 | | | 4.06578 | 0.886458 | 872 | 12 | |
| 872 12 | Peak 18 | | | 4.005712 | 0.881728 | 872 | 12 | |
| 872 12 | Peak 24 | | | 3.563666 | 0.76143 | 872 | 12 | |
| 872 12 | Peak 26 | | | 3.506188 | 0.830593 | 872 | 12 | |
| 872 12 | Peak 21 | | | 3.274746 | 0.798109 | 872 | 12 | |
| 872 12 | Peak 56 | | | 3.27061 | 0.760579 | 872 | 12 | |
| 872 12 | Peak 82 | | | 2.904232 | 0.828689 | 872 | 12 | |
| 872 12 | Peak 37 | | | 2.650426 | 0.787926 | 872 | 12 | |
| 872 12 | Peak 45 | | | 2.550487 | 0.717038 | 872 | 12 | |
| 872 12 | Peak 25 | | | 2.457691 | 0.752518 | 872 | 12 | |
| 872 12 | Peak 01 | | | 2.178114 | 0.631741 | 872 | 12 | |
| 872 12 | Peak 13 | | | 2.145886 | 0.695653 | 872 | 12 | |
| 872 12 | Peak 73 | | | 2.069574 | 0.600119 | 872 | 12 | |
| 872 12 | Peak 29 | | | 2.065815 | 0.787443 | 872 | 12 | |
| 872 12 | Peak 12 | | | 1.605227 | 0.686881 | 872 | 12 | |
| 872 12 | Peak 04 | | | 1.468949 | 0.760458 | 872 | 12 | |
| 872 12 | Peak 42 | | | 1.375671 | 0.657611 | 872 | 12 | |
| 872 12 | Peak 09 | | | 1.35681 | 0.444755 | 872 | 12 | |
| 872 12 | Peak 38 | | | 1.340273 | 0.716277 | 872 | 12 | |
| 872 12 | Peak 39 | | | 0.884817 | 0.562073 | 872 | 12 | |
| 872 12 | Peak 62 | | | 0.828732 | 0.556907 | 872 | 12 | |

TABLE 6
Group of different fragment ions from precursor m/z 873.7717 corresponding
to ionized TAG (C16:0; C18:1; C18:3) found in the SWATH ® window m/z 873

| UnitMassFile_R | Name | Cluster | Group_FSD | m.z | RT | m.z. Min | m.z. Max | RT. Min | RT. Max |
|---|---|---|---|---|---|---|---|---|---|
| 873 12 | Peak 096 | 56 | 147 | 576.5066 | 3.871434 | 576.4602 | 576.5551 | 3.711622 | 3.965811 |
| 873 12 | Peak 095 | 55 | 147 | 575.5036 | 3.871426 | 575.4642 | 575.5523 | 3.721027 | 3.965811 |
| 873 12 | Peak 104 | 60 | 147 | 600.5064 | 3.872438 | 600.4656 | 600.5556 | 3.73043 | 3.952815 |
| 873 12 | Peak 004 | 2 | 147 | 95.08664 | 3.872492 | 95.07757 | 95.09822 | 3.739835 | 3.952815 |
| 873 12 | Peak 103 | 59 | 147 | 599.5028 | 3.871793 | 599.463 | 599.5494 | 3.70692 | 3.952815 |
| 873 12 | Peak 015 | 4 | 147 | 109.1018 | 3.872344 | 109.0929 | 109.1121 | 3.807645 | 3.952815 |
| 873 12 | Peak 062 | 11 | 147 | 263.237 | 3.871988 | 263.219 | 263.2556 | 3.807645 | 3.952815 |
| 873 12 | Peak 024 | 6 | 147 | 123.1173 | 3.872534 | 123.108 | 123.1284 | 3.807645 | 3.952815 |
| 873 12 | Peak 022 | 5 | 147 | 121.1015 | 3.872322 | 121.0929 | 121.1115 | 3.807645 | 3.9385 |
| 873 12 | Peak 055 | 8 | 147 | 239.237 | 3.873971 | 239.2242 | 239.2526 | 3.807645 | 3.952815 |
| 873 12 | Peak 002 | 1 | 147 | 93.0707 | 3.872797 | 93.06372 | 93.0787 | 3.82182 | 3.952815 |
| 873 12 | Peak 058 | 9 | 147 | 245.2264 | 3.872423 | 245.2127 | 245.2414 | 3.82182 | 3.952815 |
| 873 12 | Peak 073 | 12 | 147 | 313.2735 | 3.87597 | 313.2551 | 313.29 | 3.837796 | 3.965811 |
| 873 12 | Peak 007 | 18 | 147 | 97.10178 | 3.872486 | 97.09489 | 97.1102 | 3.807645 | 3.9385 |
| 873 12 | Peak 031 | 26 | 147 | 135.117 | 3.87242 | 135.1082 | 135.1262 | 3.807645 | 3.9385 |
| 873 12 | Peak 013 | 3 | 147 | 107.0862 | 3.872884 | 107.0781 | 107.0956 | 3.82182 | 3.9385 |
| 873 12 | Peak 033 | 27 | 147 | 137.1325 | 3.871729 | 137.1239 | 137.1421 | 3.807645 | 3.9385 |
| 873 12 | Peak 063 | 11 | 147 | 264.2401 | 3.871571 | 264.2253 | 264.2551 | 3.82182 | 3.9385 |
| 873 12 | Peak 043 | 34 | 147 | 175.1483 | 3.872013 | 175.1377 | 175.1601 | 3.82182 | 3.9385 |
| 873 12 | Peak 029 | 7 | 147 | 133.1012 | 3.872965 | 133.0927 | 133.1107 | 3.82182 | 3.9385 |
| 873 12 | Peak 038 | 31 | 147 | 161.1324 | 3.871711 | 161.1227 | 161.1424 | 3.82182 | 3.9385 |
| 873 12 | Peak 035 | 29 | 147 | 149.1324 | 3.87272 | 149.1244 | 149.1417 | 3.82182 | 3.952815 |
| 873 12 | Peak 078 | 14 | 147 | 337.2737 | 3.872001 | 337.2569 | 337.2957 | 3.82182 | 3.9385 |
| 873 12 | Peak 034 | 28 | 147 | 147.117 | 3.871997 | 147.1076 | 147.1265 | 3.82182 | 3.9385 |
| 873 12 | Peak 040 | 32 | 147 | 163.1479 | 3.873655 | 163.1392 | 163.1573 | 3.82182 | 3.9385 |
| 873 12 | Peak 097 | 57 | 147 | 577.5118 | 3.875788 | 577.4875 | 577.5486 | 3.837796 | 3.922718 |
| 873 12 | Peak 020 | 24 | 147 | 119.0858 | 3.87224 | 119.0774 | 119.0943 | 3.82182 | 3.9385 |
| 873 12 | Peak 094 | 54 | 147 | 574.4915 | 3.867454 | 574.4691 | 574.5131 | 3.781879 | 3.9385 |
| 873 12 | Peak 001 | 16 | 147 | 91.05512 | 3.872972 | 91.04853 | 91.06335 | 3.837796 | 3.9385 |
| 873 12 | Peak 093 | 53 | 147 | 573.4856 | 3.870229 | 573.4614 | 573.5087 | 3.82182 | 3.9385 |
| 873 12 | Peak 005 | 2 | 147 | 96.08943 | 3.872811 | 96.0822 | 96.09742 | 3.82182 | 3.9385 |
| 873 12 | Peak 100 | 58 | 147 | 578.5212 | 3.878375 | 578.4954 | 578.5429 | 3.837796 | 3.9385 |
| 873 12 | Peak 060 | 10 | 147 | 261.2217 | 3.873471 | 261.2098 | 261.2349 | 3.837796 | 3.9385 |
| 873 12 | Peak 036 | 30 | 147 | 151.1479 | 3.871915 | 151.1393 | 151.1567 | 3.837796 | 3.9385 |
| 873 12 | Peak 012 | 21 | 147 | 105.0707 | 3.872897 | 105.0632 | 105.0791 | 3.837796 | 3.9385 |
| 873 12 | Peak 056 | 8 | 147 | 240.2406 | 3.873721 | 240.2295 | 240.2536 | 3.837796 | 3.9385 |
| 873 12 | Peak 075 | 13 | 147 | 319.2633 | 3.87153 | 319.2494 | 319.2796 | 3.837796 | 3.9385 |
| 873 12 | Peak 074 | 12 | 147 | 314.2771 | 3.875621 | 314.2652 | 314.2902 | 3.837796 | 3.9385 |
| 873 12 | Peak 050 | 39 | 147 | 189.1638 | 3.8721 | 189.1562 | 189.1737 | 3.837796 | 3.9385 |
| 873 12 | Peak 106 | 62 | 147 | 602.521 | 3.863341 | 602.5012 | 602.5428 | 3.82182 | 3.922718 |
| 873 12 | Peak 006 | 17 | 147 | 97.06612 | 3.872968 | 97.05874 | 97.07403 | 3.837796 | 3.922718 |
| 873 12 | Peak 016 | 4 | 147 | 110.1051 | 3.87427 | 110.0978 | 110.1141 | 3.837796 | 3.922718 |
| 873 12 | Peak 018 | 23 | 147 | 111.1175 | 3.870656 | 111.1102 | 111.1251 | 3.837796 | 3.922718 |
| 873 12 | Peak 059 | 9 | 147 | 246.2296 | 3.872142 | 246.2172 | 246.2416 | 3.837796 | 3.9385 |
| 873 12 | Peak 079 | 14 | 147 | 338.2778 | 3.872021 | 338.266 | 338.2893 | 3.837796 | 3.922718 |
| 873 12 | Peak 023 | 5 | 147 | 122.1054 | 3.872391 | 122.0968 | 122.114 | 3.837796 | 3.9385 |
| 873 12 | Peak 045 | 35 | 147 | 177.1633 | 3.874184 | 177.1534 | 177.1722 | 3.837796 | 3.922718 |
| 873 12 | Peak 025 | 6 | 147 | 124.1206 | 3.873682 | 124.1124 | 124.1281 | 3.837796 | 3.922718 |
| 873 12 | Peak 003 | 1 | 147 | 94.07437 | 3.874277 | 94.06727 | 94.08096 | 3.82182 | 3.9385 |
| 873 12 | Peak 008 | 19 | 147 | 99.08107 | 3.871337 | 99.07438 | 99.08843 | 3.837796 | 3.922718 |
| 873 12 | Peak 030 | 7 | 147 | 134.1049 | 3.874151 | 134.0977 | 134.1125 | 3.853772 | 3.922718 |
| 873 12 | Peak 057 | 40 | 147 | 243.2141 | 3.876235 | 243.2031 | 243.2252 | 3.853772 | 3.922718 |
| 873 12 | Peak 076 | 13 | 147 | 320.2657 | 3.871686 | 320.2565 | 320.2742 | 3.837796 | 3.922718 |
| 873 12 | Peak 061 | 10 | 147 | 262.226 | 3.873212 | 262.2169 | 262.2374 | 3.837796 | 3.922718 |
| 873 12 | Peak 017 | 22 | 147 | 111.0811 | 3.873342 | 111.0745 | 111.0879 | 3.837796 | 3.922718 |
| 873 12 | Peak 041 | 33 | 147 | 165.1631 | 3.874141 | 165.1557 | 165.1702 | 3.837796 | 3.922718 |
| 873 12 | Peak 014 | 3 | 147 | 108.0899 | 3.872573 | 108.0824 | 108.0971 | 3.853772 | 3.922718 |

| | | | | | | | | MS/MS library |
|---|---|---|---|---|---|---|---|---|
| UnitMassFile_R | Name | Library. m.z | Library. RT | Slope | RSQ | UnitMass_File | R ClusterNumber | annotation |
| 873 12 | Peak 096 | | | 69.25278 | 0.793248 | 873 | 12 | |
| 873 12 | Peak 095 | | | 36.78403 | 0.586206 | 873 | 12 | |
| 873 12 | Peak 104 | | | 30.72283 | 0.800198 | 873 | 12 | |
| 873 12 | Peak 004 | | | 20.01529 | 0.830488 | 873 | 12 | |
| 873 12 | Peak 103 | | | 13.4459 | 0.582382 | 873 | 12 | |
| 873 12 | Peak 015 | | | 12.41179 | 0.88195 | 873 | 12 | |
| 873 12 | Peak 062 | | | 10.00279 | 0.7682 | 873 | 12 | |
| 873 12 | Peak 024 | | | 8.03449 | 0.827614 | 873 | 12 | |
| 873 12 | Peak 022 | | | 5.563046 | 0.854754 | 873 | 12 | |
| 873 12 | Peak 055 | | | 5.381633 | 0.793196 | 873 | 12 | |
| 873 12 | Peak 002 | | | 4.753261 | 0.823292 | 873 | 12 | |
| 873 12 | Peak 058 | | | 4.195127 | 0.862004 | 873 | 12 | |
| 873 12 | Peak 073 | | | 4.180501 | 0.748574 | 873 | 12 | |
| 873 12 | Peak 007 | | | 3.905669 | 0.723522 | 873 | 12 | |
| 873 12 | Peak 031 | | | 3.899232 | 0.737161 | 873 | 12 | |
| 873 12 | Peak 013 | | | 3.812776 | 0.759051 | 873 | 12 | |
| 873 12 | Peak 033 | | | 3.800643 | 0.767287 | 873 | 12 | |
| 873 12 | Peak 063 | | | 3.747925 | 0.846478 | 873 | 12 | |
| 873 12 | Peak 043 | | | 3.389538 | 0.823389 | 873 | 12 | |
| 873 12 | Peak 029 | | | 3.120197 | 0.764748 | 873 | 12 | |
| 873 12 | Peak 038 | | | 2.984516 | 0.708895 | 873 | 12 | |
| 873 12 | Peak 035 | | | 2.982121 | 0.661238 | 873 | 12 | |
| 873 12 | Peak 078 | | | 2.911883 | 0.718683 | 873 | 12 | |
| 873 12 | Peak 034 | | | 2.707279 | 0.735188 | 873 | 12 | |
| 873 12 | Peak 040 | | | 2.605506 | 0.679145 | 873 | 12 | |
| 873 12 | Peak 097 | | | 2.57586 | 0.645278 | 873 | 12 | |
| 873 12 | Peak 020 | | | 2.191767 | 0.679133 | 873 | 12 | |
| 873 12 | Peak 094 | | | 2.100532 | 0.699778 | 873 | 12 | |
| 873 12 | Peak 001 | | | 2.055505 | 0.721542 | 873 | 12 | |
| 873 12 | Peak 093 | | | 1.978659 | 0.701302 | 873 | 12 | |
| 873 12 | Peak 005 | | | 1.959717 | 0.812741 | 873 | 12 | |
| 873 12 | Peak 100 | | | 1.784392 | 0.718565 | 873 | 12 | |
| 873 12 | Peak 060 | | | 1.672397 | 0.721396 | 873 | 12 | |
| 873 12 | Peak 036 | | | 1.664181 | 0.565933 | 873 | 12 | |
| 873 12 | Peak 012 | | | 1.43816 | 0.528926 | 873 | 12 | |
| 873 12 | Peak 056 | | | 1.423306 | 0.685595 | 873 | 12 | |
| 873 12 | Peak 075 | | | 1.409642 | 0.739693 | 873 | 12 | |
| 873 12 | Peak 074 | | | 1.372597 | 0.707254 | 873 | 12 | |
| 873 12 | Peak 050 | | | 1.31099 | 0.639528 | 873 | 12 | |
| 873 12 | Peak 106 | | | 1.259519 | 0.736398 | 873 | 12 | |
| 873 12 | Peak 006 | | | 1.245283 | 0.69901 | 873 | 12 | |
| 873 12 | Peak 016 | | | 1.16378 | 0.57325 | 873 | 12 | |
| 873 12 | Peak 018 | | | 1.126013 | 0.673747 | 873 | 12 | |
| 873 12 | Peak 059 | | | 1.0402 | 0.486745 | 873 | 12 | |
| 873 12 | Peak 079 | | | 0.957057 | 0.832732 | 873 | 12 | |
| 873 12 | Peak 023 | | | 0.933825 | 0.595974 | 873 | 12 | |
| 873 12 | Peak 045 | | | 0.839179 | 0.584059 | 873 | 12 | |
| 873 12 | Peak 025 | | | 0.72291 | 0.46706 | 873 | 12 | |
| 873 12 | Peak 003 | | | 0.695048 | 0.433309 | 873 | 12 | |
| 873 12 | Peak 008 | | | 0.666898 | 0.558014 | 873 | 12 | |
| 873 12 | Peak 030 | | | 0.631759 | 0.475184 | 873 | 12 | |
| 873 12 | Peak 057 | | | 0.615474 | 0.335372 | 873 | 12 | |
| 873 12 | Peak 076 | | | 0.61021 | 0.631602 | 873 | 12 | |
| 873 12 | Peak 061 | | | 0.585855 | 0.474373 | 873 | 12 | |
| 873 12 | Peak 017 | | | 0.555336 | 0.390935 | 873 | 12 | |
| 873 12 | Peak 041 | | | 0.455377 | 0.311155 | 873 | 12 | |
| 873 12 | Peak 014 | | | 0.240005 | 0.085131 | 873 | 12 | |

TABLE 7
Group of different fragment ions from precursor m/z 877.7197 corresponding
to ionized TAG (C16:0; C18:1; C18:3) found in the SWATH ® window m/z 877

| UnitMassFile_R | Name | Cluster | Group_FSD | m.z | RT | m.z. Min | m.z. Max | RT. Min | RT. Max |
|---|---|---|---|---|---|---|---|---|---|
| 877_16 | Peak_160 | 123 | 147 | 877.7253 | 3.863964 | 877.6649 | 877.7904 | 3.77765 | 3.951433 |
| 877_16 | Peak_142 | 107 | 147 | 597.4833 | 3.863912 | 597.4478 | 597.5271 | 3.807717 | 3.951433 |
| 877_16 | Peak_161 | 124 | 147 | 878.7233 | 3.864371 | 878.673 | 878.7818 | 3.807717 | 3.9298 |
| 877_16 | Peak_151 | 116 | 147 | 621.4833 | 3.865778 | 621.4349 | 621.5264 | 3.792683 | 3.940433 |
| 877_16 | Peak_135 | 102 | 147 | 575.5019 | 3.863315 | 575.4589 | 575.5435 | 3.792683 | 3.9298 |
| 877_16 | Peak_144 | 109 | 147 | 599.5018 | 3.866232 | 599.4678 | 599.5404 | 3.807717 | 3.9298 |
| 877_16 | Peak_141 | 106 | 147 | 595.4724 | 3.864101 | 595.4483 | 595.5 | 3.82605 | 3.9298 |

| | | | | | | | | MS/MS library |
|---|---|---|---|---|---|---|---|---|
| UnitMassFile_R | Name | Library. m.z | Library. RT | Slope | RSQ | UnitMass_File | R ClusterNumber | annotation |
| 877_16 | Peak_160 | | | 54.07176 | 0.743395 | 877 | 16 | |
| 877_16 | Peak_142 | | | 10.72106 | 0.551348 | 877 | 16 | |
| 877_16 | Peak_161 | | | 8.586255 | 0.575849 | 877 | 16 | |
| 877_16 | Peak_151 | | | 6.93952 | 0.501154 | 877 | 16 | |
| 877_16 | Peak_135 | | | 6.919195 | 0.514856 | 877 | 16 | |
| 877_16 | Peak_144 | | | 5.432876 | 0.550299 | 877 | 16 | |
| 877_16 | Peak_141 | | | 0.350248 | 0.151413 | 877 | 16 | |

TABLE 8
Group of different fragment ions from precursor m/z 878.7231 corresponding
to ionized TAG (C16:0; C18:1; C18:3) found in the SWATH ® window m/z 878

| UnitMassFile_R | Name | Cluster | Group_FSD | m.z | RT | m.z. Min | m.z. Max | RT. Min | RT. Max |
|---|---|---|---|---|---|---|---|---|---|
| 878_9 | Peak_185 | 128 | 147 | 878.7285 | 3.857515 | 878.6722 | 878.7978 | 3.775234 | 3.94995 |
| 878_9 | Peak_156 | 107 | 147 | 575.502 | 3.834311 | 575.4666 | 575.5445 | 3.731329 | 3.930517 |
| 878_9 | Peak_34987 | 1706 | 147 | 878.7231 | 3.85731 | 878.6946 | 878.7699 | 3.79685 | 3.925167 |
| 878_9 | Peak_102 | 72 | 147 | 313.2731 | 3.813997 | 313.2534 | 313.2934 | 3.731329 | 3.895944 |
| 878_9 | Peak_165 | 115 | 147 | 598.4861 | 3.858411 | 598.4498 | 598.5258 | 3.804994 | 3.930517 |
| 878_9 | Peak_187 | 129 | 147 | 879.7366 | 3.823029 | 879.6725 | 879.7981 | 3.760721 | 3.895944 |
| 878_9 | Peak_177 | 124 | 147 | 623.5017 | 3.829082 | 623.4681 | 623.5386 | 3.790114 | 3.913176 |
| 878_9 | Peak_095 | 68 | 147 | 287.2367 | 3.824467 | 287.2212 | 287.2547 | 3.775234 | 3.895944 |
| 878_9 | Peak_158 | 108 | 147 | 576.5043 | 3.856698 | 576.4659 | 576.5371 | 3.790114 | 3.930517 |
| 878_9 | Peak_153 | 104 | 147 | 549.4653 | 3.827072 | 549.4338 | 549.4967 | 3.775234 | 3.895944 |
| 878_9 | Peak_166 | 116 | 147 | 599.5 | 3.798096 | 599.4655 | 599.538 | 3.731329 | 3.940906 |
| 878_9 | Peak_003 | 8 | 147 | 95.08659 | 3.790892 | 95.07883 | 95.09535 | 3.716449 | 3.895944 |
| 878_9 | Peak_176 | 123 | 147 | 622.4853 | 3.859297 | 622.4464 | 622.5239 | 3.790114 | 3.940906 |
| 878_9 | Peak_085 | 63 | 147 | 269.226 | 3.823564 | 269.2122 | 269.2423 | 3.746208 | 3.895944 |
| 878_9 | Peak_081 | 60 | 147 | 267.2105 | 3.772357 | 267.1964 | 267.2241 | 3.731329 | 3.878222 |
| 878_9 | Peak_092 | 65 | 147 | 285.2212 | 3.774951 | 285.2057 | 285.2391 | 3.731329 | 3.878222 |
| 878_9 | Peak_163 | 113 | 147 | 597.4838 | 3.844999 | 597.4454 | 597.5179 | 3.731329 | 3.94995 |
| 878_9 | Peak_182 | 127 | 147 | 877.7237 | 3.857763 | 877.6892 | 877.7812 | 3.790114 | 3.940906 |
| 878_9 | Peak_175 | 122 | 147 | 621.4822 | 3.85878 | 621.4502 | 621.5065 | 3.804994 | 3.930517 |
| 878_9 | Peak_011 | 14 | 147 | 121.101 | 3.77389 | 121.0928 | 121.1099 | 3.716449 | 3.878222 |
| 878_9 | Peak_043 | 37 | 147 | 201.1632 | 3.779827 | 201.152 | 201.1741 | 3.716449 | 3.878222 |
| 878_9 | Peak_167 | 117 | 147 | 600.5035 | 3.861438 | 600.4681 | 600.5339 | 3.804994 | 3.930517 |
| 878_9 | Peak_104 | 74 | 147 | 314.2766 | 3.763572 | 314.2636 | 314.2911 | 3.731329 | 3.826889 |
| 878_9 | Peak_149 | 100 | 147 | 543.4753 | 3.780736 | 543.4582 | 543.4977 | 3.731329 | 3.844244 |
| 878_9 | Peak_044 | 38 | 147 | 203.1788 | 3.820983 | 203.169 | 203.1912 | 3.746208 | 3.895944 |
| 878_9 | Peak_002 | 7 | 147 | 93.0705 | 3.786965 | 93.0636 | 93.07858 | 3.716449 | 3.878222 |
| 878_9 | Peak_039 | 33 | 147 | 187.147 | 3.776528 | 187.137 | 187.1583 | 3.716449 | 3.861233 |
| 878_9 | Peak_144 | 95 | 147 | 525.4666 | 3.781736 | 525.4425 | 525.4878 | 3.746208 | 3.844244 |
| 878_9 | Peak_008 | 12 | 147 | 109.1013 | 3.793133 | 109.0929 | 109.1091 | 3.731329 | 3.895944 |
| 878_9 | Peak_010 | 13 | 147 | 119.0858 | 3.789915 | 119.0774 | 119.0943 | 3.716449 | 3.878222 |
| 878_9 | Peak_038 | 32 | 147 | 185.1325 | 3.794739 | 185.1227 | 185.1438 | 3.731329 | 3.878222 |
| 878_9 | Peak_030 | 26 | 147 | 175.1487 | 3.786398 | 175.1398 | 175.1584 | 3.731329 | 3.895944 |
| 878_9 | Peak_109 | 78 | 147 | 337.2727 | 3.776207 | 337.2579 | 337.2864 | 3.716449 | 3.861233 |
| 878_9 | Peak_015 | 18 | 147 | 135.1174 | 3.770973 | 135.1082 | 135.1263 | 3.716449 | 3.861233 |
| 878_9 | Peak_155 | 106 | 147 | 574.49 | 3.758959 | 574.4715 | 574.5155 | 3.716449 | 3.878222 |
| 878_9 | Peak_154 | 105 | 147 | 567.4741 | 3.827198 | 567.4492 | 567.4997 | 3.790114 | 3.895944 |
| 878_9 | Peak_093 | 66 | 147 | 286.2246 | 3.763377 | 286.2126 | 286.2365 | 3.731329 | 3.819311 |
| 878_9 | Peak_013 | 16 | 147 | 131.0859 | 3.79042 | 131.0779 | 131.0941 | 3.731329 | 3.878222 |
| 878_9 | Peak_029 | 25 | 147 | 173.1317 | 3.77253 | 173.1225 | 173.1411 | 3.702303 | 3.861233 |
| 878_9 | Peak_040 | 34 | 147 | 189.1631 | 3.805289 | 189.1545 | 189.1739 | 3.731329 | 3.878222 |
| 878_9 | Peak_083 | 61 | 147 | 268.2145 | 3.762167 | 268.2033 | 268.2264 | 3.731329 | 3.819311 |
| 878_9 | Peak_152 | 103 | 147 | 548.4521 | 3.76528 | 548.4285 | 548.4747 | 3.731329 | 3.826889 |
| 878_9 | Peak_007 | 11 | 147 | 107.0854 | 3.778175 | 107.078 | 107.0941 | 3.702303 | 3.878222 |
| 878_9 | Peak_075 | 55 | 147 | 261.221 | 3.762732 | 261.2104 | 261.2333 | 3.702303 | 3.826889 |
| 878_9 | Peak_147 | 98 | 147 | 541.4604 | 3.7633 | 541.4396 | 541.479 | 3.688156 | 3.826889 |
| 878_9 | Peak_146 | 97 | 147 | 531.4548 | 3.828797 | 531.4357 | 531.4747 | 3.790114 | 3.878222 |
| 878_9 | Peak_012 | 15 | 147 | 123.1173 | 3.792605 | 123.108 | 123.1252 | 3.716449 | 3.895944 |
| 878_9 | Peak_042 | 36 | 147 | 199.1478 | 3.788642 | 199.1391 | 199.1571 | 3.731329 | 3.878222 |
| 878_9 | Peak_024 | 22 | 147 | 159.1164 | 3.781876 | 159.1081 | 159.1259 | 3.731329 | 3.861233 |
| 878_9 | Peak_150 | 101 | 147 | 547.4524 | 3.764366 | 547.4306 | 547.4768 | 3.731329 | 3.819311 |
| 878_9 | Peak_148 | 99 | 147 | 542.4632 | 3.714485 | 542.4418 | 542.4846 | 3.67511 | 3.775234 |
| 878_9 | Peak_021 | 21 | 147 | 147.117 | 3.773769 | 147.1094 | 147.1249 | 3.702303 | 3.861233 |
| 878_9 | Peak_142 | 93 | 147 | 524.4529 | 3.720764 | 524.4335 | 524.469 | 3.688156 | 3.775234 |
| 878_9 | Peak_140 | 91 | 147 | 523.4468 | 3.771877 | 523.4286 | 523.4642 | 3.702303 | 3.844244 |
| 878_9 | Peak_018 | 19 | 147 | 145.1003 | 3.782155 | 145.0928 | 145.1081 | 3.716449 | 3.861233 |
| 878_9 | Peak_181 | 126 | 147 | 861.7288 | 3.817764 | 861.7004 | 861.7626 | 3.760721 | 3.878222 |
| 878_9 | Peak_006 | 10 | 147 | 105.0706 | 3.789568 | 105.0631 | 105.0791 | 3.731329 | 3.861233 |
| 878_9 | Peak_048 | 41 | 147 | 211.1484 | 3.773821 | 211.1408 | 211.1572 | 3.731329 | 3.844244 |
| 878_9 | Peak_001 | 6 | 147 | 91.05512 | 3.787642 | 91.0484 | 91.06322 | 3.731329 | 3.878222 |
| 878_9 | Peak_068 | 53 | 147 | 243.2105 | 3.796177 | 243.2015 | 243.2213 | 3.731329 | 3.878222 |
| 878_9 | Peak_051 | 44 | 147 | 215.1792 | 3.793856 | 215.1696 | 215.1883 | 3.731329 | 3.878222 |
| 878_9 | Peak_031 | 27 | 147 | 183.1161 | 3.779188 | 183.1078 | 183.1269 | 3.731329 | 3.861233 |
| 878_9 | Peak_025 | 23 | 147 | 161.1331 | 3.78467 | 161.1246 | 161.1407 | 3.716449 | 3.878222 |
| 878_9 | Peak_028 | 24 | 147 | 171.1178 | 3.819231 | 171.1077 | 171.1262 | 3.790114 | 3.878222 |
| 878_9 | Peak_057 | 49 | 147 | 225.1639 | 3.76622 | 225.1553 | 225.1722 | 3.731329 | 3.826889 |
| 878_9 | Peak_049 | 42 | 147 | 213.1633 | 3.784508 | 213.1556 | 213.1721 | 3.731329 | 3.878222 |
| 878_9 | Peak_014 | 17 | 147 | 133.1015 | 3.817807 | 133.0928 | 133.1107 | 3.790114 | 3.878222 |
| 878_9 | Peak_067 | 52 | 147 | 241.1935 | 3.780276 | 241.1849 | 241.2046 | 3.731329 | 3.878222 |
| 878_9 | Peak_111 | 79 | 147 | 339.2894 | 3.78686 | 339.275 | 339.3036 | 3.731329 | 3.861233 |
| 878_9 | Peak_074 | 54 | 147 | 259.2416 | 3.821682 | 259.2317 | 259.2522 | 3.746208 | 3.878222 |
| 878_9 | Peak_041 | 35 | 147 | 197.1318 | 3.769399 | 197.1225 | 197.1403 | 3.731329 | 3.861233 |
| 878_9 | Peak_180 | 125 | 147 | 860.7174 | 3.745909 | 860.6938 | 860.7435 | 3.702303 | 3.804994 |
| 878_9 | Peak_101 | 71 | 147 | 311.2587 | 3.750492 | 311.248 | 311.2705 | 3.731329 | 3.810756 |
| 878_9 | Peak_076 | 56 | 147 | 263.2359 | 3.832205 | 263.2265 | 263.2471 | 3.790114 | 3.913176 |

| | | | | | | | | MS/MS library |
|---|---|---|---|---|---|---|---|---|
| UnitMassFile_R | Name | Library. m.z | Library. RT | Slope | RSQ | UnitMass_File | R ClusterNumber | annotation |
| 878_9 | Peak_185 | | | 28.54901 | 0.641565 | 878 | 9 | |
| 878_9 | Peak_156 | | | 9.000761 | 0.826404 | 878 | 9 | |
| 878_9 | Peak_34987 | | | 8.37732 | 0.184197 | 878 | 9 | |
| 878_9 | Peak_102 | | | 5.617092 | 0.857945 | 878 | 9 | |
| 878_9 | Peak_165 | | | 4.830378 | 0.575122 | 878 | 9 | |
| 878_9 | Peak_187 | | | 4.602802 | 0.73347 | 878 | 9 | |
| 878_9 | Peak_177 | | | 4.542057 | 0.839878 | 878 | 9 | |
| 878_9 | Peak_095 | | | 4.172384 | 0.648164 | 878 | 9 | |
| 878_9 | Peak_158 | | | 3.862101 | 0.571887 | 878 | 9 | |
| 878_9 | Peak_153 | | | 3.759027 | 0.852243 | 878 | 9 | |
| 878_9 | Peak_166 | | | 3.721662 | 0.730546 | 878 | 9 | |
| 878_9 | Peak_003 | | | 3.517092 | 0.821883 | 878 | 9 | |
| 878_9 | Peak_176 | | | 3.454481 | 0.441553 | 878 | 9 | |
| 878_9 | Peak_085 | | | 3.407861 | 0.653587 | 878 | 9 | |
| 878_9 | Peak_081 | | | 3.187624 | 0.786994 | 878 | 9 | |
| 878_9 | Peak_092 | | | 2.991929 | 0.773354 | 878 | 9 | |
| 878_9 | Peak_163 | | | 2.692481 | 0.486736 | 878 | 9 | |
| 878_9 | Peak_182 | | | 2.39565 | 0.418473 | 878 | 9 | |
| 878_9 | Peak_175 | | | 2.365048 | 0.572112 | 878 | 9 | |
| 878_9 | Peak_011 | | | 2.27257 | 0.705086 | 878 | 9 | |
| 878_9 | Peak_043 | | | 2.209394 | 0.76129 | 878 | 9 | |
| 878_9 | Peak_167 | | | 2.095452 | 0.413064 | 878 | 9 | |
| 878_9 | Peak_104 | | | 2.024129 | 0.780325 | 878 | 9 | |
| 878_9 | Peak_149 | | | 2.006757 | 0.783624 | 878 | 9 | |
| 878_9 | Peak_044 | | | 1.984985 | 0.562508 | 878 | 9 | |
| 878_9 | Peak_002 | | | 1.978044 | 0.635027 | 878 | 9 | |
| 878_9 | Peak_039 | | | 1.966418 | 0.831638 | 878 | 9 | |
| 878_9 | Peak_144 | | | 1.885139 | 0.680814 | 878 | 9 | |
| 878_9 | Peak_008 | | | 1.851608 | 0.686438 | 878 | 9 | |
| 878_9 | Peak_010 | | | 1.725185 | 0.795146 | 878 | 9 | |
| 878_9 | Peak_038 | | | 1.686165 | 0.872747 | 878 | 9 | |
| 878_9 | Peak_030 | | | 1.683696 | 0.700254 | 878 | 9 | |
| 878_9 | Peak_109 | | | 1.65465 | 0.646305 | 878 | 9 | |
| 878_9 | Peak_015 | | | 1.618849 | 0.638725 | 878 | 9 | |
| 878_9 | Peak_155 | | | 1.588785 | 0.657524 | 878 | 9 | |
| 878_9 | Peak_154 | | | 1.576639 | 0.762665 | 878 | 9 | |
| 878_9 | Peak_093 | | | 1.568873 | 0.696361 | 878 | 9 | |
| 878_9 | Peak_013 | | | 1.551368 | 0.792663 | 878 | 9 | |
| 878_9 | Peak_029 | | | 1.537852 | 0.785346 | 878 | 9 | |
| 878_9 | Peak_040 | | | 1.53342 | 0.631329 | 878 | 9 | |
| 878_9 | Peak_083 | | | 1.533303 | 0.810258 | 878 | 9 | |
| 878_9 | Peak_152 | | | 1.532656 | 0.757799 | 878 | 9 | |
| 878_9 | Peak_007 | | | 1.471265 | 0.659494 | 878 | 9 | |
| 878_9 | Peak_075 | | | 1.411686 | 0.815415 | 878 | 9 | |
| 878_9 | Peak_147 | | | 1.351312 | 0.745145 | 878 | 9 | |
| 878_9 | Peak_146 | | | 1.334909 | 0.650003 | 878 | 9 | |
| 878_9 | Peak_012 | | | 1.301469 | 0.759779 | 878 | 9 | |
| 878_9 | Peak_042 | | | 1.272915 | 0.649571 | 878 | 9 | |
| 878_9 | Peak_024 | | | 1.261134 | 0.865246 | 878 | 9 | |
| 878_9 | Peak_150 | | | 1.247716 | 0.643644 | 878 | 9 | |
| 878_9 | Peak_148 | | | 1.231051 | 0.668006 | 878 | 9 | |
| 878_9 | Peak_021 | | | 1.189006 | 0.629161 | 878 | 9 | |
| 878_9 | Peak_142 | | | 1.11328 | 0.624617 | 878 | 9 | |
| 878_9 | Peak_140 | | | 1.112728 | 0.748668 | 878 | 9 | |
| 878_9 | Peak_018 | | | 1.103084 | 0.646817 | 878 | 9 | |
| 878_9 | Peak_181 | | | 1.099954 | 0.555157 | 878 | 9 | |
| 878_9 | Peak_006 | | | 1.094349 | 0.765057 | 878 | 9 | |
| 878_9 | Peak_048 | | | 1.07485 | 0.754965 | 878 | 9 | |
| 878_9 | Peak_001 | | | 1.068979 | 0.771862 | 878 | 9 | |
| 878_9 | Peak_068 | | | 1.059611 | 0.672125 | 878 | 9 | |
| 878_9 | Peak_051 | | | 1.051353 | 0.762873 | 878 | 9 | |
| 878_9 | Peak_031 | | | 1.044773 | 0.662217 | 878 | 9 | |
| 878_9 | Peak_025 | | | 1.042241 | 0.69465 | 878 | 9 | |
| 878_9 | Peak_028 | | | 1.031129 | 0.514764 | 878 | 9 | |
| 878_9 | Peak_057 | | | 1.019374 | 0.72591 | 878 | 9 | |
| 878_9 | Peak_049 | | | 0.976027 | 0.644274 | 878 | 9 | |
| 878_9 | Peak_014 | | | 0.974815 | 0.461478 | 878 | 9 | |
| 878_9 | Peak_067 | | | 0.948367 | 0.635757 | 878 | 9 | |
| 878_9 | Peak_111 | | | 0.943612 | 0.562513 | 878 | 9 | |
| 878_9 | Peak_074 | | | 0.882837 | 0.538278 | 878 | 9 | |
| 878_9 | Peak_041 | | | 0.863172 | 0.617934 | 878 | 9 | |
| 878_9 | Peak_180 | | | 0.77966 | 0.743357 | 878 | 9 | |
| 878_9 | Peak_101 | | | 0.738124 | 0.612976 | 878 | 9 | |
| 878_9 | Peak_076 | | | 0.714093 | 0.513403 | 878 | 9 | |

TABLE 9
Group of different fragment ions from precursor m/z 893.6948 corresponding
to ionized TAG (C16:0; C18:1; C18:3) found in the SWATH ® window m/z 893

| UnitMassFile_R | Name | Cluster | Group_FSD | m.z | RT | m.z. Min | m.z. Max | RT. Min | RT. Max |
|---|---|---|---|---|---|---|---|---|---|
| 893_11 | Peak_165 | 91 | 147 | 893.7555 | 3.965755 | 893.6854 | 893.7824 | 3.84698 | 4.057746 |

| | | | | | | | | MS/MS library |
|---|---|---|---|---|---|---|---|---|
| UnitMassFile_R | Name | Library. m.z | Library. RT | Slope | RSQ | UnitMass_File | R ClusterNumber | annotation |
| 893_11 | Peak_165 | | | 0.865254 | 0.337808 | 893 | 11 | |

Discovery of Unknown Compounds

The invention is capable of producing groups like in the example shown above for substances that are not included in the library. Groups containing ion species of good quality (based on the correlation coefficient) that were not annotated with the library are potential unknowns compounds.

Since SWATH® acquisition yields structural information through fragments and exact masses, it is possible to perform structure elucidation activities based on the information provided by the invention. When the presence of an unknown has been confirmed the exact mass of the full scan ion, the retention time and the list of fragments will be entered in the library. The advantage of the method is that this step can be performed in silico without the need of re-injecting the sample since the selectivity of the method provides sufficient quality for recording good MS/MS spectrum by minimizing the risk of obtaining mixed fragmentation spectrum from several precursors compared to methods using SWATH® windows larger than 1 Da

Setting Up High Throughput Method

The example shown above demonstrates how the invention is used to annotate several hundreds of ion species. The result of this process is a list of known metabolite and candidate unknown's compounds. The correlation and the slope allow the user to assess the analysis quality and the abundance of a related metabolite.

Known substances with sufficient quality are transferred to the high throughput method for examples on a triple quadrupole instrument. The use of scheduled MRM allows for the analysis of 1000s of compounds simultaneously.

The list of fragments obtained from SWATH® experiments is used to produce the MRM method as it is known which fragments are the most abundant and yield the best correlation with the different volume of material. Thus, a list of MRM transition can be produced even for unknown compounds with no redundancy which increase the number of metabolites included in a single method (i.e. with 1 Da SWATH® windows we eliminate the risk of using two MRM transitions that will measure the same metabolite).

### REFERENCES

1. The statistical approach selected was to fit a simple linear regression model (SLRM) Reference: Chambers, J. M. (1992) Linear models. Chapter 4 of Statistical Models in S. ed J. M. Chambers and T. J. Hastie, Wadsworth & Brooks/Cole.
2. Wilkinson, G. N. and Rogers, C. E. (1973) Symbolic descriptions of factorial models for analysis of variance. Applied Statistics, 22, 392-9.
3. Karl Pearson (20 Jun. 1895) “Notes on regression and inheritance in the case of two parents,” Proceedings of the Royal Society of London, 58: 240-242.
4. R Core Team (2016). R: A language and environment for statistical computing. R Foundation for Statistical Computing, Vienna, Austria. URL https://www.R-project.org/.
5. http://127.0.0.1:11877/library/stats/html/lm.html
6. Wang, H. and Song, M. (2011) Ckmeans.1d.dp: optimal k-means clustering in one dimension by dynamic programming. The R Journal 3(2), 29-33.

1. A method of analyzing the metabolic content of a biological sample comprising:
i) providing one or more samples of extracted metabolites from the biological sample;
ii) performing a chromatography coupled mass spectrometry analysis of the extracted metabolites to generate a full raw data set for full scan ions;
iii) generating a full data cluster set from the full raw data set obtained in step ii) by grouping full scan ions according to isotope and adduct values;
iv) performing a tandem mass spectrometry analysis of the extracted metabolites with a plurality of mass selection windows to generate a raw SWATH® data set for fragment ions;
v) generating a SWATH® data cluster set from the raw SWATH® data set obtained in step iv) by grouping fragment ions according to retention time and mass values;
vi) aligning the SWATH® data cluster set with the full data cluster set to generate characteristic profile for each extracted metabolite;
vii) comparing the characteristic profile of each extracted metabolite obtained in step vi) with a reference library of characteristic profiles of metabolites to provide the metabolic content of the biological sample.

2. The method of claim 1 wherein a raw SWATH® data set for fragment ions comprises mass and retention time and intensity data.

3. The method of claim 1 wherein a SWATH® data cluster set comprises mass, retention time, fragment and intensity data.

4. The method of claim 1 wherein the characteristic profile for each extracted metabolite of step (vi) comprises (a) mass, retention time, isotope and adduct values and intensity of full scan ions, and (b) mass, retention time, fragment and intensity data for the fragment ions.

5. The method of claim 1 wherein the reference library of step vii) comprises predetermined characteristic profiles of predetermined metabolites.

6. The method of claim 5 wherein the predetermined characteristic profiles of predetermined metabolites are determined from authentic standards of the known compounds, from an analysis of samples containing the compounds, from existing spectral libraries, or computationally generated by applying empirical or a priori fragmentation or modification rules to the known compounds.

7. The method of claim 5 wherein step vii) comprises comparing said predetermined characteristic profile of predetermined metabolites to the characteristic profile of each extracted metabolite to assign the extracted metabolite to a predetermined metabolite.

8. The method of claim 7 wherein step vii) comprises calculating a score that represents how well the predetermined characteristic profile of predetermined metabolites and characteristic profile of each extracted metabolite match.

9. The method of claim 1 wherein a plurality of samples of extracted metabolites from the biological sample are analyzed.

10. The method of claim 9 wherein the samples of extracted metabolites are derived from different amounts of biological sample.

11. The method of claim 10 wherein a simple linear regression model (SLRM) analysis is generated for the full raw data set and SWATH® data cluster set.

12. The method of claim 1 wherein each mass selection window of the plurality of mass selection windows has a width less than approximately 5 Daltons, preferably approximately 1 Dalton.

13. The method of claim 1 wherein each mass selection window of the plurality of mass selection windows has a width of approximately 1 Da.

14. The method of claim 1 wherein the chromatography coupled mass spectrometry analysis in step ii) is performed by liquid chromatography and/or by gas chromatography.

15. The method of claim 1 wherein the chromatography coupled mass spectrometry analysis in step iv) is performed using MS/MS, preferably QToF.

16. A method of identifying a metabolic signature of a biological sample comprising:
i) providing two or more samples of extracted metabolites derived from different amounts of the biological sample;
ii) performing a chromatography coupled mass spectrometry analysis of the extracted metabolites to generate a full raw data set for full scan ions;
iii) generating a full data cluster set from the full raw data set obtained in step ii) by grouping full scan ions according to isotope and adduct values;
iv) performing a tandem mass spectrometry analysis of the extracted metabolites with a plurality of mass selection windows to generate a raw SWATH® data set for fragment ions;
v) generating a SWATH® data cluster set from the raw SWATH® data set obtained in step iv) by grouping fragment ions according to retention time and mass values;
vi) aligning the SWATH® data cluster set with the full data cluster set to generate characteristic profile for each extracted metabolite;
vii) comparing the characteristic profile of each extracted metabolite obtained in step vi) with a reference library of characteristic profiles of metabolites to provide the metabolic content of the biological sample;
viii) performing a simple linear regression model (SLRM) analysis for the full raw data set and SWATH® data cluster set to generate a SLRM value for the metabolite
ix) selecting those metabolites which have a SLRM correlation coefficient of at least 0.7 as the signature.

17. A high-throughput screening method of analyzing metabolites in a biological sample comprising performing the method of claim 16 and analyzing the signature metabolites in the biological sample.

18. The method of claim 1 wherein the said biological sample is a sample of a bodily fluid, preferably a blood, plasma, lymph or serum sample, or is a tissue sample, preferably a sample of liver tissue, heart tissue, prostate tissue, pancreas tissue, brain tissue, kidney tissue, adipose tissue, gut, skeleton tissue, lung tissue, bladder, breast tissue, cecum and/or skin tissue, such as dermal layer, comprising the epidermis and/or corium and/or subcutis.

19. The method of claim 1 wherein the said biological sample is a sample of an algae or plant, preferably of a monocotyledonous or dicotyledonous plant or is a tissue sample, preferably leaf tissue, root tissue, shoot tissue, stem tissue, reproductive tissue (for example a flower tissue or pollen) and/or seed tissue and/or liquid comprising exudate thereof and/or volatile compounds released thereof.
